(19) United States
(12) Patent Application Publication
(10) Pub. No.: US 2019/0030230 A1
(43) Pub. Date: Jan. 31, 2019

(54) Wearable and Implanted Closed Loop System for Human Circulatory Assistance

(71) Applicant: Robert A. Connor, St. Paul, MN (US)

(72) Inventor: Robert A. Connor, St. Paul, MN (US)

(73) Assignee: Medibotics LLC, St. Paul, MN (US)

(21) Appl. No.: 16/150,469

(22) Filed: Oct. 3, 2018

Related U.S. Application Data
(63) Continuation-in-part of application No. 15/418,620, filed on Jan. 27, 2017.
Application No. 15/418,620 is a continuation-in-part of application No. 14/951,475, filed on Nov. 24, 2015.
Application No. 14/951,475 is a continuation-in-part of application No. 13/901,131, filed on May 23, 2013, now Pat. No. 9,536,449.
Application No. 13/901,131 is a continuation-in-part of application No. 14/071,112, filed on Nov. 4, 2013, now abandoned.
Application No. 14/071,112 is a continuation-in-part of application No. 14/623,337, filed on Feb. 16, 2015, now Pat. No. 9,582,035.
Application No. 14/623,337 is a continuation-in-part of application No. 14/459,937, filed on Aug. 14, 2014.
(60) Provisional application No. 62/297,827, filed on Feb. 20, 2016.
(60) Provisional application No. 62/439,147, filed on Dec. 26, 2016.
(60) Provisional application No. 62/245,311, filed on Oct. 23, 2015.
(60) Provisional application No. 61/866,583, filed on Aug. 16, 2013.

(51) Int. Cl. A61M 1/12; A61B 5/00; A61B 5/026

(52) U.S. Cl. CPC A61M 1/122; A61B 5/0031; A61B 5/026; A61B 5/6824; A61B 2562/046; A61B 5/4836; A61B 5/6815; A61B 5/6825; A61B 5/6829; A61B 2562/0233

(57) ABSTRACT

This invention is a closed-loop system for human circulatory assistance comprising one or more wearable devices which collect data on a biometric parameter concerning a person's body in real time and one or more implanted circulatory assistance devices whose operation is adjusted in real time based on analysis of the data on the biometric parameter. This system can selectively improve blood circulation, either overall or to selected body regions, in order to prevent tissue degradation, promote wound healing, and maintain proper organ functioning.

### CROSS-REFERENCE TO RELATED APPLICATIONS

This patent application:

(A) is a continuation in part of U.S. patent application Ser. No. 15/418,620 by Robert A. Connor entitled “Integrated System for Managing Cardiac Rhythm Including Wearable and Implanted Devices” filed on Jan. 27, 2017 which: (1) is a continuation in part of U.S. patent application Ser. No. 14/951,475 by Robert A. Connor entitled “Wearable Spectroscopic Sensor to Measure Food Consumption Based on Interaction Between Light and the Human Body” filed on Nov. 24, 2015 which: (a) is a continuation in part of U.S. patent application Ser. No. 13/901,131 (now U.S. Pat. No. 9,536,449) by Robert A. Connor entitled “Smart Watch and Food Utensil for Monitoring Food Consumption” filed on May 23, 2013; (b) is a continuation in part of U.S. patent application Ser. No. 14/071,112 by Robert A. Connor entitled “Wearable Spectroscopy Sensor to Measure Food Consumption” filed on Nov. 4, 2013; (c) is a continuation in part of U.S. patent application Ser. No. 14/623,337 (now U.S. Pat. No. 9,582,035) by Robert A. Connor entitled “Wearable Computing Devices and Methods for the Wrist and/or Forearm” filed on Feb. 16, 2015; and (d) claims the priority benefit of U.S. provisional patent application 62/245,311 by Robert A. Connor entitled “Wearable Device for the Arm with Close-Fitting Biometric Sensors” filed on Oct. 23, 2015; (2) claims the priority benefit of U.S. provisional patent application 62/297,827 by Robert A. Connor entitled “System for Automatic Adjustment of Cardiac Function Based on Data from a Wearable Biometric Sensor” filed on Feb. 20, 2016; and (3) claims the priority benefit of U.S. provisional patent application 62/439,147 by Robert A. Connor entitled “Arcuate Wearable Device for Measuring Body Hydration and/or Glucose Level” filed on Dec. 26, 2016; and

(B) is a continuation in part of U.S. patent application Ser. No. 14/459,937 by Robert A. Connor entitled “Implanted Extracardiac Device for Circulatory Assistance” filed on Aug. 14, 2014 which claims the priority benefit of U.S. Provisional Patent Application No. 61/866,583 by Robert A. Connor entitled “Stent for Actively Accelerating Blood Flow” filed on Aug. 16, 2013.

The entire contents of these related applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to systems for human circulatory assistance.

### INTRODUCTION

Proper blood circulation and oxygenation for tissue in body extremities is important for physiological functioning and tissue health. Various factors, including pathology and exercise, change oxygen levels in body extremities. It would be desirable to have a closed loop system for human circulatory assistance which can selectively increase blood flow to body extremities in response to low oxygen levels in those extremities. This can help to improve physiological functioning, extremity tissue health, promote wound healing, and potentially even avoid amputation. This is the unmet clinical need which is addressed by this invention.

### REVIEW OF THE PRIOR ART

U.S. Patent Applications 20050115561 (Stahmann et al., Jun. 2, 2005, “Patient Monitoring, Diagnosis, and/or Therapy Systems and Methods”) and 20110061647 (Stahmann et al., Mar. 17, 2011, “Patient Monitoring, Diagnosis, and/or Therapy Systems and Methods”) and U.S. Pat. No. 7,787,946 (Stahmann et al., Aug. 31, 2010, “Patient Monitoring, Diagnosis, and/or Therapy Systems and Methods”) disclose cooperative communication between an implantable cardiac function device and an external respiratory therapy device.

U.S. Pat. No. 8,515,548 (Rofougaran et al., Aug. 20, 2013, “Article of Clothing Including Bio-Medical Units”) discloses clothing with a plurality of bio-medical units for physical therapy. U.S. Patent Application 20060195039 (Drew et al., Aug. 31, 2006, “Clustering with Combined Physiological Signals”) and U.S. Pat. No. 8,768,446 (Drew et al., Jul. 1, 2014, “Clustering with Combined Physiological Signals”) disclose the generation of an extended cluster of data for activation of implantable systems such as those that provide stimulation and drug delivery, pacemaker systems, defibrillator systems, and cochlear implant systems.

U.S. Patent Application 20160018347 (Drbal et al., Jan. 21, 2016, “Designs, Systems, Configurations, and Methods for Immittance Spectroscopy”) discloses the use of immittance spectroscopy to identify the composition of liquids. U.S. Patent Application 20140316479 (Taff et al., Oct. 23, 2014, “Implantable Medical Device”) discloses a leadless pacemaker which may include a spectroscopic sensor. U.S. Pat. No. 8,463,345 (Kuhn et al., Jun. 11, 2013, “Device and Method for Monitoring of Absolute Oxygen Saturation and Total Hemoglobin Concentration”), U.S. Pat. No. 8,634,890 (Kuhn et al., Jan. 21, 2014, “Device and Method for Monitoring of Absolute Oxygen Saturation and Tissue Hemoglobin Concentration”), and U.S. Pat. No. 8,666,466 (Kuhn et al., Mar. 4, 2014, “Device and Method for Monitoring of Absolute Oxygen Saturation and Tissue Hemoglobin Concentration”) disclose an implanted oxygen saturation monitor. U.S. Pat. No. 8,428,729 (Schwartz et al., Apr. 23, 2013, “Cardiac Stimulation Apparatus and Method for the Control of Hypertension”) discloses changing cardiac rhythm based on changes in blood pressure.

U.S. Pat. No. 8,112,148 (Giftakis et al., Feb. 7, 2012, “System and Method for Monitoring Cardiac Signal Activity in Patients with Nervous System Disorders”) discloses the use of brain event information to interpret cardiac signals. U.S. Patent Application 20040131998 (Marom et al., Jul. 8, 2004, “Cerebral Programming”) and U.S. Pat. No. 7,499,894 (Marom et al., Mar. 3, 2009, “Cerebral Programming”) disclose training a biological neural network to control an insulin pump or a pacemaker. U.S. Patent Applications 20050081847 (Lee et al., Apr. 21, 2005, “Automatic Activation of Medical Processes”) and 20100106211 (Lee et al., Apr. 29, 2010, “Automatic Activation of Medical Processes”) and U.S. Pat. No. 7,668,591 (Lee et al., Feb. 23, 2010, “Automatic Activation of Medical Processes”), U.S. Pat. No. 7,668,591 (Lee et al., Feb. 23, 2010, “Automatic Activation of Medical Processes”), and U.S. Pat. No. 8,380,296 (Lee et al., Feb. 19, 2013, “Automatic Activation of Medical Processes”) disclose changing cardiac rhythm therapy based on brain state information.

U.S. Patent Applications 20070260286 (Giftakis et al., Nov. 8, 2007, “System and Method for Utilizing Brain State Information to Modulate Cardiac Therapy”) and 20070265677 (Giftakis et al., Nov. 15, 2007, “System and Method for Utilizing Brain State Information to Modulate Cardiac Therapy”) and U.S. Pat. No. 8,209,019 (Giftakis et al., Jun. 26, 2012, “System and Method for Utilizing Brain State Information to Modulate Cardiac Therapy”) and U.S. Pat. No. 8,214,035 (Giftakis et al., Jul. 3, 2012, “System and Method for Utilizing Brain State Information to Modulate Cardiac Therapy”) disclose changing cardiac therapy based on brain state information.

### SUMMARY OF THE INVENTION

This invention can be embodied in a system for human circulatory assistance comprising: a wearable device which is configured to be worn by a person, wherein the wearable device collects data on a biometric parameter concerning the person's body in real time; and an implanted circulatory assistance device which is configured to be implanted within the person's body, wherein the implanted circulatory assistance device assists in management of the person's cardiac rhythm and/or assists in pumping the person's blood, and wherein the operation of the implanted circulatory assistance device is controlled and/or adjusted in real time based on analysis of the biometric parameter. This system can help to prevent tissue degradation, can promote wound healing, and may even help to avoid amputation.

In an example, a wearable device can be a finger ring, smart watch, wrist band, ear ring, earlobe clip, ankle band, or smart sock. In an example, a biometric parameter can be body oxygenation level. In an example, an implanted circulatory assistance device can be an implanted cardiac pacemaker, an implanted central (heart assist) blood pump, or an implanted non-central (peripheral) blood pump. In an example, a system for human circulatory assistance can comprise a plurality of wearable devices and plurality of implanted non-central (peripheral) blood pumps which enables independent adjustment of circulatory assistance for different portions of a person's body based on biometric parameter values from those different body portions.

### INTRODUCTION TO THE FIGURES

FIG. 1 shows a system for human circulatory assistance with a wearable device and a cardiac pacemaker.

FIG. 2 shows a system for human circulatory assistance with a wearable device and a central blood pump.

FIG. 3 shows a system for human circulatory assistance with a first wearable device at a first location, a second wearable device at a second location, a first non-central blood pump, and a second non-central blood pump.

### DETAILED DESCRIPTION OF THE FIGURES

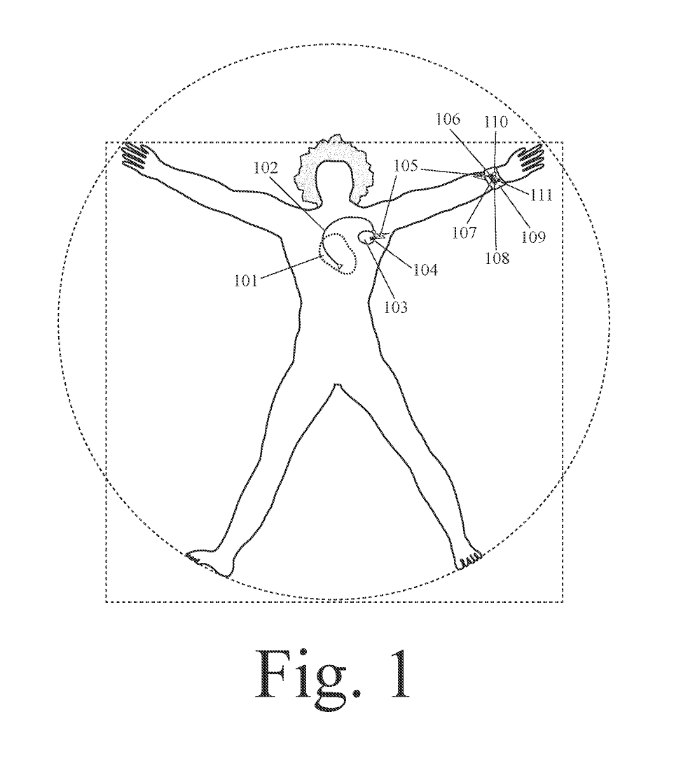

FIGS. 1 through 3 show some examples of how a wearable and implanted closed loop system for human circulatory assistance can be embodied, but these examples do not limit the generalizability of the claims. Before discussing the specific examples shown in FIGS. 1 through 3, the following section is an introduction to key concepts and component variations of this invention. These key concepts and component variations can be applied to the examples shown in FIGS. 1 through 3, but they are not repeated in the narratives accompanying each figure in order avoid narrative redundancy.

This invention is a partially or fully closed-loop system for human circulatory assistance whose operation is adjusted in real time based on analysis of data concerning one or more biometric parameters collected by one or more wearable sensors. In an example, human circulatory assistance can be provided by an implanted cardiac pacemaker whose operation is adjusted in real time based on overall body oxygenation level. In an example, human circulatory assistance can be provided by an implanted central (heart assist) blood pump whose operation is adjusted in real time based on overall body oxygenation level. In an example, human circulatory assistance can be provided by a plurality of non-central (peripheral) blood pumps in different body regions whose operations are individually adjusted based on oxygenation levels in those respective body regions.

Automatic adjustment of cardiac functioning and/or blood circulation in real time in response to abnormal biometric values measured by wearable devices can help to maintain healthy biological processes and prevent tissue degradation. For example, detection of low overall body oxygenation level by wearable sensors can trigger increased systemic blood flow. For example, detection of low oxygenation levels in specific portions of the body by wearable sensors can trigger selected increased blood flow to those specific portions. This can help to prevent tissue degradation, promote wound healing, and maintain proper organ functioning.

In an example, a system for human circulatory assistance can comprise: a wearable device which is configured to be worn by a person, wherein the wearable device collects data on a biometric parameter concerning the person's body in real time; and an implanted circulatory assistance device which is configured to be implanted within the person's body, wherein the implanted circulatory assistance device assists in management of the person's cardiac rhythm and/or assists in pumping the person's blood, and wherein operation of the implanted circulatory assistance device is controlled and/or adjusted in real time based on analysis of the data on the biometric parameter.

In an example, a system for human circulatory assistance can comprise: a finger ring, smart watch, smart watch band, wrist band, ankle band, smart sock, ear ring, ear bud, or smart patch worn by a person, wherein the finger ring, smart watch, smart watch band, wrist band, ankle band, smart sock, ear ring, ear bud, or smart patch collects data on a biometric parameter concerning the person's body; and an implanted cardiac pacemaker or implanted blood pump, wherein operation of the implanted cardiac pacemaker or implanted blood pump is controlled and/or adjusted based on analysis of the data on the biometric parameter.

In an example, a system for human circulatory assistance can comprise: a finger ring, smart watch, smart watch band, wrist band, ankle band, smart sock, ear ring, ear bud, or smart patch worn by a person, wherein the finger ring, smart watch, smart watch band, wrist band, ankle band, smart sock, ear ring, ear bud, or smart patch collects data concerning the person's body oxygenation level; and an implanted cardiac pacemaker, wherein operation of the implanted cardiac pacemaker is adjusted based on the person's body oxygenation level in one or more ways selected from the group consisting of: a change in the voltage of electromagnetic energy delivered to the heart to stimulate contractions, a change in the degree of coordination and/or timing between electromagnetic energy stimulation of different heart chambers, a change in the frequency of electromagnetic energy stimulation of heart contractions, a change in the location(s) on the heart where electromagnetic energy is delivered, a change in the magnitude of heart contractions which are stimulated, and a change in the regularity of heart contractions which are stimulated.

In an example, a system for human circulatory assistance can comprise: a finger ring, smart watch, smart watch band, wrist band, ankle band, smart sock, ear ring, ear bud, or smart patch worn by a person, wherein the finger ring, smart watch, smart watch band, wrist band, ankle band, smart sock, ear ring, ear bud, or smart patch collects data concerning the person's body oxygenation level; and an implanted blood pump, wherein operation of the implanted blood pump is adjusted based on the person's body oxygenation level in one or more ways selected from the group consisting of: activation or deactivation of the pump in order to increase or decrease blood flow; an increase or decrease in the duration of pump operation in order to increase or decrease blood flow; a increase or decrease in the speed of pump rotation, undulation, compression, and/or contraction in order to increase or decrease blood flow; and an increase or decrease in the magnitude of pump undulation, compression, and/or contraction in order to increase or decrease blood flow.

In an example, a wearable device of this system can have an optical sensor. In an example, an optical sensor can be a spectroscopic sensor. In an example, a wearable device can have a sensor which is in optical communication with body tissue, fluid, and/or gas selected from the group consisting of: blood, interstitial fluid, lymphatic fluid, sweat, tears, aqueous humour, saliva, exhaled gas, capillaries, blood vessels, skin, fatty tissue, muscles, and nerves. In an example, a wearable device of this system can have an electromagnetic energy sensor. In an example, an electromagnetic sensor can measure the conductivity, resistance, impedance, capacitance, and/or permittivity of body tissue and/or fluid. In an example, a wearable device can have a sensor which is in electromagnetic communication with body tissue, fluid, and/or gas which is selected from the group consisting of: blood, interstitial fluid, lymphatic fluid, sweat, tears, aqueous humour, saliva, exhaled gas, capillaries, blood vessels, skin, fatty tissue, muscles, bones, and nerves.

In an example, a wearable device of this system can be worn on a person's finger. In an example, a wearable device of this system can be a finger ring with embedded biometric sensors. In an example, biometric sensors can be spectroscopic sensors. In an example, a wearable device of this system can be a finger sleeve made from elastic fabric with embedded biometric sensors. In an example, sensors to collect data on a biometric parameter can be located on the inner (e.g. closest to body) surface of a finger ring or finger sleeve.

In an example, a plurality of sensors can be distributed around (at least half of) the inner circumference of a finger ring or finger sleeve. In an example, a plurality of light-energy emitters and receivers can be distributed around (at least half of) the circumference of a finger ring or finger sleeve. In an example, an alternating sequence of light-energy emitters and receivers can be distributed around (at least half of) the circumference of a finger ring or finger sleeve. In an example, a plurality of electromagnetic energy emitters and receivers can be distributed around (at least half of) the circumference of a finger ring or finger sleeve. In an example, an alternating sequence of electromagnetic energy emitters and receivers can be distributed around (at least half of) the circumference of a finger ring or finger sleeve.

In an example, a wearable device of this system can be worn on a person's wrist or forearm. In an example, a wearable device can be a smart watch with embedded biometric sensors. In an example, biometric sensors can be spectroscopic sensors. In an example, there can be sensors in the housing (e.g. the primary display housing) of a smart watch, around the band of a smart watch, or both. In an example, a wearable device can be a fitness band, bracelet, bangle with embedded biometric sensors. In an example, a biometric sensor can be located in a primary housing of a wrist-worn device (such as a smart watch), wherein the primary housing is worn on the dorsal side of a person's wrist. In an example, a biometric sensor can be located in a secondary housing of a wrist-worn device, wherein the secondary housing is worn on the ventral side of the wrist.

In an example, sensors to collect data on a biometric parameter can be distributed around (at least half of) the circumference of a watch band, wrist band, fitness band, or bracelet. In an example, a plurality of light-energy emitters and receivers can be distributed around (at least half of) the circumference of a watch band, wrist band, fitness band, or bracelet. In an example, an alternating sequence of light-energy emitters and receivers can be distributed around (at least half of) the circumference a watch band, wrist band, fitness band, or bracelet. In an example, a plurality of electromagnetic energy emitters and receivers can be distributed around (at least half of) the circumference of a watch band, wrist band, fitness band, or bracelet. In an example, an alternating sequence of electromagnetic energy emitters and receivers can be distributed around the circumference a watch band, wrist band, fitness band, or bracelet.

In an example, a wearable device of this system can be worn on a person's ear or inserted into a person's ear canal. In an example, a wearable device can be an ear ring, earlobe clip, ear bud, ear plug, hearing aid, or ear-worn speaker/microphone with embedded biometric sensors. In an example, a wearable device can be an ear ring, earlobe clip, ear bud, ear plug, hearing aid, or ear-worn speaker/microphone with embedded spectroscopic sensors. In an example, a wearable device can be an ear ring, earlobe clip, ear bud, ear plug, hearing aid, or ear-worn speaker/microphone with embedded electromagnetic energy sensors. In an example, an ear ring with embedded biometric sensors can be attached to a person's earlobe through a pierced opening in the ear lobe. In an example, an ear ring can be attached to a person's earlobe by pressure (e.g. a clamp or clip). In an example, an ear ring can be attached to a person's earlobe by magnetic attraction of members on opposite sides of the earlobe.

In an example, a sensor to collect data on a biometric parameter can be located on the dorsal and/or proximal side of an ear lobe. In an example, a sensor to collect data on a biometric parameter can be located on the ventral and/or distal side of an ear lobe. In an example, a light-energy emitter can be on one side (e.g. the dorsal or proximal side) of an ear lobe and a light-energy receiver can be on the opposite side (e.g. the ventral and/or distal side) of the ear lobe. In an example, an electromagnetic energy emitter can be on one side of an ear lobe and an electromagnetic energy receiver can be on the opposite side of the ear lobe. In an example, a longitudinal array of sensors can be distributed along an ear bud or ear plug which is inserted into a person's ear canal. In an example, a circumferential array of sensors can be distributed around an ear bud or ear plug which is inserted into a person's ear canal.

In an example, a wearable device of this system can be worn on a person's ankle or foot. In an example, a wearable device can be an ankle band or a smart sock with embedded biometric sensors. In an example, biometric sensors can be spectroscopic sensors or electromagnetic energy sensors. In an example, biometric sensors can be distributed around (at least half of) the circumference of an ankle band or smart sock. In an example, biometric sensors can be woven into at least half of the circumference of an ankle band or smart sock. In an example, a plurality of light-energy emitters and receivers can be distributed around (at least half of) the circumference of an ankle band or smart sock. In an example, an alternating sequence of light-energy emitters and receivers can be distributed around the circumference an ankle band or smart sock. In an example, a plurality of electromagnetic energy emitters and receivers can be distributed around (at least half of) the circumference of an ankle band or smart sock. In an example, an alternating sequence of electromagnetic energy emitters and receivers can be distributed around the circumference an ankle band or smart sock.

In an example, a wearable device of this system can be eyewear. In an example, a wearable device of this system can be eyeglasses with embedded biometric sensors. In an example, these biometric sensors can be optical (e.g. spectroscopic) sensors. In an example, these biometric sensor can be electromagnetic (e.g. electroencephalographic) sensors. In an example, eyewear can comprise a plurality of biometric sensors on the frame of the eyewear. In an example, eyewear can comprise a plurality of biometric sensors on the sidepieces (e.g. the “temples”) of the eyewear. In an example, eyewear can comprise a plurality of biometric sensors on the front piece and/or nose bridge of the eyewear. In an example, a wearable device can be a contact lens with embedded optical or electromagnetic energy sensors to measure a biometric parameter.

In an example, a wearable device can be temporarily and removably adhered to a person's skin. In an example, a wearable device can be a smart adhesive patch and/or an electronically-functional adhesive patch with biometric sensors. In an example, these biometric sensors can be spectroscopic sensors. In an example, spectroscopic sensors in a smart adhesive patch and/or an electronically-functional adhesive patch can be used to monitor the molecular composition of a person's sweat and/or gases emitted from the person's skin. In an example, a wearable device can be a temporary smart tattoo with biometric sensors. In an example, a wearable device can be an electronically-functional tattoo with biometric sensors. In an example, a wearable device can be a permanent smart tattoo and/or a permanent electronically-functional tattoo with embedded biometric sensors.

In an example, a wearable device of this system can be worn on a person's leg. In an example, a wearable device can be a leg band with embedded biometric sensors. In an example, a wearable device of this system can be worn on a person's foot. In an example, a wearable device can be an ankle band, smart sock, foot pad, or toe ring. In an example, a wearable device of this system can be worn on a person's upper arm. In an example, a wearable device can be an arm band or elbow sleeve with embedded biometric sensors. In an example, a wearable device of this system can be worn on a person's torso. In an example, a wearable device can be a waist belt, a chest band, an adhesive patch, or an electronic tattoo. In an example, a wearable device of this system can be worn on a person's head. In an example, a wearable device can be a headband, an intra-oral appliance, or a nose ring.

In an example, a wearable device can be selected from the group consisting of: finger ring, wrist watch (housing, band, or both), wrist band (e.g. fitness band), pin, and earlobe clip. In an example, a wearable device of this system can be selected from the group consisting of: necklace or pendant, hair comb or band, earpiece, bracelet or bangle, earring, skull cap, Augmented Reality (AR) eyewear, electronically-functional eyewear, wrist strap, buckle, sleeve, face mask or goggles, ear bud, and finger nail attachment.

In an example, a wearable device of this system can have a form which is selected from the group consisting of: headphones or headset, chest strap, contact lens, finger sleeve, hearing aid, Virtual Reality (VR) eyewear, ear plug or buds, and helmet. In an example, a wearable device of this system can have a form which is selected from the group consisting of: waist band, ear ring, visor, armband, nose ring, ear-worn Bluetooth device, finger tip thimble, knee brace, earphone, hair clip, artificial finger nail, belt or waist strap, and leg band. In an example, a wearable device can have a form which is selected from the group consisting of: smart finger ring, smart watch housing and/or band, fitness band, upper arm band, ankle band, smart sock, smart eyeglasses, smart contact lens, smart ear ring, and ear bud.

In an example, a wearable device of this system can be an article of clothing or clothing accessory with biometric sensors. In an example, these biometric sensors can be spectroscopic sensors or electromagnetic energy sensors. In an example, biometric sensors can be attached to, embedded into, woven into, sewn into, or printed onto an article of clothing or clothing accessory. In an example, an article of clothing or clothing accessory can be a short-sleeve shirt or a long-sleeve shirt. In an example, an article of clothing or clothing accessory can be a pair of shorts or pants. In an example, an article of clothing or clothing accessory can be a bra, an undershirt, or a underpants.

In an example, an article of clothing or clothing accessory with biometric sensors can be a smart sock or shoe. In an example, an article of clothing or clothing accessory can be a finger ring, finger sleeve, finger nail attachment, or glove. In an example, an article of clothing or clothing accessory can be a hat, baseball cap, skull cap, or hair comb. In an example, an article of clothing or clothing accessory can be a button, snap, or zipper. In an example, this article of clothing or clothing accessory can be a collar or cuff. In an example, this article of clothing or clothing accessory can be a belt or strap.

In an example, a wearable device of this system can comprise optical sensors (e.g. light-energy emitters and receivers) which are embedded in (or attached to) an article of clothing or clothing accessory. In an example, sensors to measure a biometric parameter can be formed by a plurality of optically-transmissive threads, yarns, fibers, or layers in an article of clothing or clothing accessory. In an example, sensors to measure a biometric parameter concerning a person's body can be formed by a grid or matrix of optically-transmissive threads, yarns, fibers, or layers in an article of clothing or clothing accessory. In an example, sensors to measure a biometric parameter concerning a person's body can be a woven grid or matrix of optically-transmissive threads, yarns, fibers, or layers in an article of clothing or clothing accessory. In an example, sensors to measure a biometric parameter concerning a person's body can be a pattern of optically-transmissive pathways which are printed onto an article of clothing or clothing accessory using optically-transmissive ink.

In an example, a wearable device of this system can comprise electromagnetic sensors which are embedded in (or attached to) an article of clothing or clothing accessory. In an example, sensors to measure a biometric parameter can be formed by a plurality of electroconductive threads, yarns, fibers, or layers in an article of clothing or clothing accessory. In an example, sensors to measure a biometric parameter concerning a person's body can be formed by a grid or matrix of electroconductive threads, yarns, fibers, or layers in an article of clothing or clothing accessory. In an example, sensors to measure a biometric parameter concerning a person's body can be a woven grid or matrix of electroconductive threads, yarns, fibers, or layers in an article of clothing or clothing accessory. In an example, sensors to measure a biometric parameter concerning a person's body can be a pattern of electromagnetic pathways which is printed onto an article of clothing or clothing accessory using electroconductive ink.

In an example, an implanted circulatory assistance device of this system can be selected from the group consisting of: cardiac rhythm management (CRM) device such as a cardiac pacemaker or implantable cardioverter-defibrillator (ICD); central (heart-assist) blood pump such as a left ventricular assist device (LVAD); and non-central (peripheral) blood pump. In an example, an implanted circulatory assistance device of this system can have a first (e.g. “feedback”) operational mode wherein its operation is adjusted in real time based on values of a biometric parameter which are measured by a wearable device and a second (“stand alone”) operational mode when the wearable device is either not being worn or is not working properly. In an example, a system can detect when a wearable device is not being worn or not working properly by a lack of biometric data, gaps in biometric data, or biometric parameter values which are outside defined bounds.

In an example, an implanted circulatory assistance device of this system can be a cardiac pacemaker which is in electromagnetic communication with a person's heart. In an example, an implanted circulatory assistance device can be a cardiac pacemaker which delivers periodic electromagnetic energy pulses to a person's heart in order to stimulate and/or regulate contraction of heart muscles. In an example, a cardiac pacemaker can deliver electromagnetic energy pulses to the heart via wires and/or leads. In an example, a cardiac pacemaker can be implanted within the heart, wherein it directly delivers electromagnetic energy pulses to the heart walls.

In an example, a closed loop system for human circulatory assistance can comprise: a wearable device which is worn by a person, wherein the wearable device collects data on a biometric parameter (such as body oxygenation level); and an implanted cardiac pacemaker, wherein operation of the cardiac pacemaker is controlled and/or adjusted based on analysis of the data on the biometric parameter. In an example, the operation of a cardiac pacemaker can be controlled and/or adjusted in one or more ways selected from the group consisting of: a change in the voltage of electromagnetic energy delivered to the heart to stimulate contractions, a change in the degree of coordination and/or timing between electromagnetic energy stimulation of different heart chambers, a change in the frequency of electromagnetic energy stimulation of heart contractions, a change in the location(s) on the heart where electromagnetic energy is delivered, a change in the magnitude of heart contractions which are stimulated, a change in the regularity of heart contractions which are stimulated, and delivery of a non-periodic electromagnetic shock to the heart to disrupt fibrillation.

In an example, one or more operating parameters of a cardiac pacemaker which are adjusted by this system can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

In an example, one or more operating parameters of a cardiac pacemaker which are adjusted by this system can be selected from the group consisting of: increase in heart electromagnetic stimulation voltage; increase in the degree of coordination and/or timing between stimulations to different heart chambers; increase in the frequency of heart contraction stimulations; change in the locations on the heart to which electromagnetic energy is delivered; increase in the magnitude of heart contraction stimulations; increase in the regularity of heart contraction stimulations; and more precise coordination of contraction of different heart chambers.

In an example, a closed loop system for human circulatory assistance can comprise: a finger ring, wherein the finger ring collects data concerning a biometric parameter (such as body oxygenation level); and an implanted cardiac pacemaker, wherein operation of the cardiac pacemaker is controlled and/or adjusted based on analysis of the data on the biometric parameter. In an example, a closed loop system for human circulatory assistance can comprise: a smart watch (including the watch band and/or watch housing) or wrist band, wherein the smart watch or wrist band collects data concerning a biometric parameter; and an implanted cardiac pacemaker; wherein operation of the cardiac pacemaker is controlled and/or adjusted based on analysis of the data on the biometric parameter. In an example, a closed loop system for human circulatory assistance can comprise: an ear ring or earlobe clip, wherein the ear ring or earlobe clip collects data concerning a biometric parameter; and an implanted cardiac pacemaker, wherein operation of the cardiac pacemaker is controlled and/or adjusted based on analysis of the data on the biometric parameter.

In an example, an implanted circulatory assistance device of this system can be an implanted central (heart-assist) blood pump which assists the heart in pumping blood. In an example, an implanted blood pump can be a Left Ventricular Assist Device (LVAD). In an example, an implanted blood pump can have a rotating impellor. In an example, an implanted blood pump can comprise a rotating helical impellor. In an example, an implanted blood pump can be an Archimedes pump. In an example, an implanted blood pump can comprise rotating arcuate fins, vanes, or blades. In an example, an implanted blood pump can be a centripetal (or, old school, “centrifugal”) pump. In an example, an implanted blood pump can be a pump with a compression chamber between two one-way valves.

In an example, an implanted blood pump can be a peristaltic pump. In an example, an implanted blood pump can be an axial pump. In an example, an implanted blood pump can be a hydroelastic pump. In an example, an implanted blood pump can be a longitudinal-membrane-wave pump. In an example, an implanted blood pump can be a magnetic flux pump. In an example, an implanted blood pump can be an elastomeric pump. In an example, an implanted blood pump can have an oscillating impellor. In an example, an implanted blood pump can be a pump with electromagnetically-driven magnetic impeller. In an example, an implanted blood pump can be an electromagnetic field pump.

In an example, an implanted blood pump can be an entrainment pump. In an example, an implanted blood pump can be a pump with fluid jets which entrain native blood flow. In an example, an implanted blood pump can be a compressive pump. In an example, an implanted blood pump can be a diaphragm pump. In an example, an implanted blood pump can be a pump with a series of circumferentially-compressive members. In an example, an implanted blood pump can be a balloon pump. In an example, an implanted blood pump can be a pulsatile flow pump. In an example, an implanted blood pump can be a continuous flow pump. In an example, an implanted blood pump can be a piston pump.

In an example, a closed loop system for human circulatory assistance can comprise: a wearable device which is worn by a person, wherein the wearable device collects data on a biometric parameter (such as body oxygenation level); and an implanted central (heart-assist) blood pump, wherein operation of the central (heart-assist) blood pump is adjusted based on analysis of the data on the biometric parameter which is collected by the wearable device. In an example, the operation of an central (heart-assist) blood pump can be adjusted in one or more ways selected from the group consisting of: activation or deactivation of the pump in order to increase or decrease blood flow; an increase or decrease in the duration of pump operation in order to increase or decrease blood flow; a increase or decrease in the speed of a pump's rotation, undulation, compression, or contraction (depending on type of pump) in order to increase or decrease blood flow; and an increase or decrease in the magnitude of pump undulation, compression, or contraction (depending on type of pump) in order to increase or decrease blood flow.

In an example, a closed loop system for human circulatory assistance can comprise: a finger ring, wherein the finger ring collects data concerning a biometric parameter (such as body oxygenation level); and a central (heart-assist) blood pump which is implanted within the person's body; wherein operation of the central (heart-assist) blood pump is adjusted based on analysis of the data on the biometric parameter. In an example, a closed loop system for human circulatory assistance can comprise: a smart watch (or wrist band), wherein the smart watch (or wrist band) collects data concerning a biometric parameter; and a central (heart-assist) blood pump which is implanted within the person's body; wherein operation of the central (heart-assist) blood pump is adjusted based on analysis of the data on the biometric parameter. In an example, a closed loop system for human circulatory assistance can comprise: an ear ring or earlobe clip, wherein the ear ring or earlobe clip collects data concerning a biometric parameter; and a central (heart-assist) blood pump which is implanted within the person's body; wherein operation of the central (heart-assist) blood pump is adjusted based on analysis of the data on the biometric parameter.

In an example, an implanted circulatory assistance device of this system can be a non-central (peripheral) blood pump which assists in pumping blood to a selected localized (e.g. peripheral) portion of a person's body. In an example, a non-central (peripheral) blood pump can have a rotating impellor. In an example, a non-central (peripheral) blood pump can comprise a rotating helical impellor. In an example, a non-central (peripheral) blood pump can be an Archimedes pump. In an example, a non-central (peripheral) blood pump can comprise rotating arcuate fins, vanes, or blades. In an example, a non-central (peripheral) blood pump can be a centripetal (or, old school, “centrifugal”) pump. In an example, a non-central (peripheral) blood pump can be a pump with a compression chamber between two one-way valves.

In an example, a non-central (peripheral) blood pump can be a peristaltic pump. In an example, an implanted blood pump of this system can be an axial pump. In an example, a non-central (peripheral) blood pump can be a hydroelastic pump. In an example, a non-central (peripheral) blood pump can be a longitudinal-membrane-wave pump. In an example, a non-central (peripheral) blood pump can be a magnetic flux pump. In an example, a non-central (peripheral) blood pump can be an elastomeric pump. In an example, a non-central (peripheral) blood pump can have an oscillating impellor. In an example, a non-central (peripheral) blood pump can be a pump with electromagnetically-driven magnetic impeller. In an example, a non-central (peripheral) blood pump can be an electromagnetic field pump.

In an example, a non-central (peripheral) blood pump can be an entrainment pump. In an example, a non-central (peripheral) blood pump can be a pump with fluid jets which entrain native blood flow. In an example, a non-central (peripheral) blood pump can be a compressive pump. In an example, a non-central (peripheral) blood pump can be a diaphragm pump. In an example, a non-central (peripheral) blood pump can be a pump with a series of circumferentially-compressive members. In an example, a non-central (peripheral) blood pump can be a balloon pump. In an example, a non-central (peripheral) blood pump can be a pulsatile flow pump. In an example, a non-central (peripheral) blood pump can be a continuous flow pump. In an example, a non-central (peripheral) blood pump can be a piston pump.

In an example, a non-central (peripheral) blood pump can be endovascularly inserted and then expanded within a peripheral blood vessel in order to provide localized circulatory assistance. In an example, a non-central (peripheral) blood pump can be endovascularly inserted and then expanded within a peripheral blood vessel in order to help pump blood to a selected peripheral portion of a person's body. In an example, a non-central (peripheral) blood pump can be spliced into a person's vasculature “in series” with a natural blood vessel. In an example, a non-central (peripheral) blood pump which is spliced into a person's vasculature “in series” replaces a segment of a natural blood vessel. In an example, a non-central (peripheral) blood pump can be spliced into a person's vasculature “in series” with a natural blood vessel in order to help pump blood to a selected peripheral portion of a person's body.

In an example, a non-central (peripheral) blood pump can be spliced into a person's vasculature “in parallel” with a natural vessel. In an example, a non-central (peripheral) blood pump can have a first end which is connected to an upstream portion of a blood vessel, a second end which is connected to a downstream portion of a blood vessel, and a blood-flow-increasing mechanism located between the two ends. In addition, a one-way valve can be inserted into the natural vessel between the upstream connection and the downstream connection.

In an example, a closed loop system for human circulatory assistance can comprise: a wearable device which is worn by a person, wherein the wearable device collects data on a biometric parameter (such as body oxygenation level); and an implanted non-central (peripheral) blood pump, wherein operation of the implanted non-central (peripheral) blood pump is adjusted based on analysis of the data on the biometric parameter which is collected by the wearable device. In an example, the operation of a non-central (peripheral) blood pump can be adjusted in one or more ways selected from the group consisting of: activation or deactivation of the pump in order to increase or decrease blood flow; an increase or decrease in the duration of pump operation in order to increase or decrease blood flow; a increase or decrease in the speed of a pump's rotation, undulation, compression, or contraction (depending on type of pump) in order to increase or decrease blood flow; an increase or decrease in the magnitude of pump undulation, compression, or contraction (depending on type of pump) in order to increase or decrease blood flow; selective operational changes in a sub-set of a plurality of non-central (peripheral) blood pumps to change blood flow in a selected sub-set of peripheral body locations.

In an example, a closed loop system for human circulatory assistance can comprise: a finger ring, wherein the finger ring collects data concerning a biometric parameter; and a non-central (peripheral) blood pump which is implanted within the person's body; wherein operation of the non-central (peripheral) blood pump is adjusted based on analysis of the data on the biometric parameter. In an example, a closed loop system for human circulatory assistance can comprise: a smart watch (or wrist band), wherein the smart watch (or wrist band) collects data concerning a biometric parameter; and a non-central (peripheral) blood pump which is implanted within the person's body; wherein operation of the non-central (peripheral) blood pump is adjusted based on analysis of the data on the biometric parameter. In an example, a closed loop system for human circulatory assistance can comprise: an ear ring or earlobe clip, wherein the ear ring or earlobe clip collects data concerning a biometric parameter; and a non-central (peripheral) blood pump which is implanted within the person's body; wherein operation of the non-central (peripheral) blood pump is adjusted based on analysis of the data on the biometric parameter.

In an example, an implanted circulatory assistance device of this system can be a single central (heart-assist) blood pump. In an example, an implanted circulatory assistance device of this system can be multiple non-central (peripheral) blood pumps. In an example, a plurality of non-central (peripheral) blood pumps can be implanted in a distributed manner in different peripheral blood vessels throughout a person's body. In an example, multiple non-central (peripheral) blood pumps can form a distributed network which provides extracardiac circulatory assistance. In an example, distributed circulatory assistance can selectively increase blood circulation to body regions or organs with the greatest (short-term or long-term) need.

In an example, a plurality of non-central (peripheral) blood pumps can comprise a fluid network of “mini-hearts” which support a person's heart only to the extent which is needed during a period of cardiac healing and recovery. In an example, a plurality of extracardiac circulatory assistance devices can comprise an efficient and effective system of distributed circulatory assistance to maintain cardiac functioning and allow cardiac healing for people with CHS. In an example, one or more implanted blood pumps can supplement, but not replace, native blood circulation. This can reduce cardiac workload until the heart recovers or for the long-term if recovery is not possible. In an example, one or more implanted blood pumps can reduce cardiac workload without completely replacing cardiac function so that the heart may still heal and recover function—avoiding the eventual need for heart transplantation or a more-invasive full-cardiac-function replacement device. In an example, multiple non-central (peripheral) blood pumps can be configured in parallel flow or in series flow.

In an example, an implanted blood pump can be configured to increase the flow of blood from an upstream location to a downstream location in a person's vasculature. In an example, the blood pump can transduce electromagnetic energy into kinetic energy. In an example, an implanted blood pump can increase the rate, speed, volume, and/or consistency of blood flow. In an example, an implanted blood pump can also improve hemodynamics. In an example, a blood pump can be structurally designed to avoid low-flow areas that can cause thrombogenesis. In an example, a blood pump can be designed to produce hemodynamic patterns that minimize thrombogenesis.

Blood flow pumps are sometimes categorized as either pulsatile or continuous. Generally, a pulsatile pump is considered to be one which produces variation in flow speed and/or pressure which is synchronized to be in phase, or out of phase, with the native cardiac pumping cycle. In an example, a blood pump can be copulsating with respect to the cardiac pumping cycle. In an example, a blood pump can be counterpulsating with respect to the cardiac pumping cycle. Pulsatile flow can be preferred for perfusion of some organs and can also help to reduce thrombogenesis. In an example, the blood pump of this invention can produce pulsatile blood flow and/or supplement native pulsatile blood flow. In an example, a control unit of this system can change a blood pump from a pulsatile flow to a continuous flow.

In an example, an implanted blood pump can have a low cross-sectional profile when it is not in operation and a high cross-sectional profile when it is in operation. This can allow an implanted blood pump to substantively supplement blood circulation when the mechanism is in operation, but to not substantively hinder native blood flow when the blood pump is not in operation. In an example, the blood pump can be defined to be “in operation” when it is actively transducing electromagnetic energy (such as from a battery or other electrical power source) into kinetic energy (in the form of blood flow). In an example, the ability to supplement native circulation when power is available without hindering native circulation when power is unavailable (or limited) can enable greater patient mobility and improved quality of life. This ability can also help to preserve the possibility of healing and recovery for the heart by only providing circulatory assistance when needed.

In an example, an implanted blood pump can produce a continuous blood flow. The designation of “continuous” can mean that a blood pump is actually intended to operate all the time, but more generally it can mean that a blood pump produces a blood flow which is not pulsatile when the pump is in operation. In other words, a continuous blood flow pump has a relatively-uniform flow speed and/or pressure as long as the pump is in operation. This distinction is important for supplemental circulation assistance devices which do not cause adverse outcomes if they are turned off (or lose power) for periods of time. Accordingly, this distinction is important for the invention disclosed herein which does not have to be in operation all the time. In an example, a continuous blood flow pump can contribute a sub-stream of continuous blood flow which is in addition to (and/or entrains) native pulsatile blood flow. In an example, the blood pump of this invention can produce and contribute a continuous blood flow when it is in operation, but it does not have to be in operation all the time. In an example, the blood pump of this invention can be hybrid pump which is capable of producing either a pulsatile or continuous blood flow. In an example, the operation of a blood pump and the type of blood flow (e.g. pulsatile or continuous) which it produces can be controlled by a control unit for the blood pump which will be discussed later in greater depth.

In an example, an implanted blood pump can be a rotary implanted blood pump. In an example, an implanted blood pump can move blood by means of a rotating impeller or turbine. In an example, an implanted blood pump can have a rotating impellor or turbine which is further comprised of one or more vanes, fins, blades, projections, winglets, airfoils, helical members, or grooves. In an example, these one or more vanes, fins, blades, projections, winglets, airfoils, or helical members can have a (first) retracted or contracted configuration in which they have a first amount of cross-sectional interaction with blood flow. In an example, these one or more vanes, fins, blades, projections, winglets, airfoils, or helical members can have a (second) protracted or expanded configuration in which they have second amount of cross-sectional interaction with blood flow. In an example, the second amount is greater than the first amount. In an example, the one or more vanes, fins, blades, projections, winglets, airfoils, helical members, or grooves transition to the second configuration when the implanted blood pump is in operation. In an example, the one or more vanes, fins, blades, airfoils, or helical members can be reversibly, repeatedly, and post-operatively moved back and forth from the first configuration to the second configuration.

In an example, an implanted blood pump can be an axial rotary pump. In an example, an implanted blood pump can comprise one or more vanes, fins, blades, projections, winglets, airfoils, or helical members which rotate around an axis which is coaxial with the longitudinal axis of the blood flow lumen, with the directional vector of native blood flow, or both. In an example, an implanted blood pump can comprise one or more vanes, fins, blades, projections, winglets, airfoils, or helical members which rotate around an axis which is substantially parallel with the longitudinal axis of the blood flow lumen, with the directional vector of native blood flow, or both. In an example, an implanted blood pump can comprise one or more vanes, fins, blades, projections, winglets, airfoils, or helical members which rotate around an axis which is substantially perpendicular to the longitudinal axis of the blood flow lumen, with the directional vector of native blood flow, or both.

In an example, an implanted blood pump can comprise a rotating helical or screw-shaped impeller. In an example, an implanted blood pump can comprise a rotating impeller with multiple helical or partial-helical members. In an example, a rotary pump can have one or more members which are rotated by a direct drive mechanical connection to an electromagnetic motor or other mechanical actuator. In an example, a rotary pump can have one or more magnetic members which are rotated by magnetic interaction with an electromagnetic field. In an example, a rotary implanted blood pump can have hydrodynamic or magnetic bearings.

In an example, an implanted blood pump can further comprise one or more moving members which increase blood flow by frictionally engaging blood and/or by entraining native blood flow. In an example, these one or more moving members can be selected from the group consisting of: airfoils, blades, fins, flippers, grooves, helical structures, rotors, threads, vanes, and winglets. In an example, the one or more moving members can have a first configuration wherein they have a first level of frictional engagement with blood flow. In an example, this first configuration can comprise being relatively close to (or flush with) a central rotating axle. In an example, this first configuration can comprise being relatively close to (or flush with) the walls of the implanted blood flow lumen. In an example, the one or more moving members can have a second configuration in which they have a second level of frictional engagement with blood flow. In an example, the second level can be substantially greater than the first level. In an example, “substantially greater” means at least 10% greater. In an example, “substantially greater” means at least 25% greater. In an example, “substantially greater” means at least 100% greater.

In an example, an implanted blood pump can move blood using peristaltic motion. In an example, an implanted blood pump can comprise a peristaltic pump. In an example, an implanted blood pump can move blood by sequential compression of the lumen by a longitudinally rolling member which rolls longitudinally and compressively (from upstream to downstream) along the walls of the lumen. In an example, an implanted blood pump can move blood by the sequential contraction (from upstream to downstream) of a series of circumferential members such as contracting bands or rings along the longitudinal axis of an implanted blood flow lumen. In an example, an implanted blood pump can move blood by sequentially inflating and deflating a series of inflatable members such as toroidal balloons along the longitudinal axis (from upstream to downstream) of an implanted blood flow lumen. In an example, an implanted blood pump can comprise a series of waving cilia-form members which wave along a lumen wall like a crowd of fans in a microscale sport arena. In an example, an implanted blood pump can move blood by propagating a longitudinal wave or pulse (such as a pressure wave) longitudinally (from upstream to downstream) along a flexible membrane (or other surface) which is in fluid communication with blood in an implanted blood flow lumen.

In an example, a wearable device such as a smart finger ring, a smart watch, a smart wrist band, a smart ear ring, or smart eyewear for collecting data on a biometric parameter can have a spectroscopic sensor. (A spectroscopic sensor can also be called a “spectroscopy sensor.”) In an example, a spectroscopic sensor can further comprise a light-energy emitter (e.g. a light source) and a light-energy receiver (e.g. a photodetector). In an example, the light-energy receiver can receive light-energy from the light-energy emitter after that light-energy has been transmitted through body tissue and/or fluid or has been reflected by body tissue and/or fluid. Different types of molecules absorb or reflect different wavelengths of light by different amounts. Accordingly, analysis of changes in the spectrum of light-energy which has interacted with body tissue and/or fluid can be used to estimate the molecular composition of that body tissue and/or fluid. In an example, a wearable device can perform photoplethysmography (PPG).

In an example, a light-energy receiver of a spectroscopic sensor can receive light-energy which has been transmitted through body tissue and/of fluid. In an example, transmission of light-energy through body tissue and/or fluid changes the spectrum of that light-energy and this change in spectrum is analyzed to get information about the composition of that body tissue and/or fluid. In an example, light-energy from a light-energy emitter on a first side of a body member (such as a finger or earlobe) can be directed toward the body member, transmitted through the body member, and then received by a light-energy receiver on another side (e.g. the diametrically-opposite side) of the body member. In an example, changes in the spectrum of light which has been transmitted through the body tissue and/or fluid of the body member can be analyzed to estimate the value of a biometric parameter or changes in that value over time.

In an example, a light-energy receiver of a spectroscopic sensor can receive light-energy which has been reflected by body tissue and/of fluid. In an example, reflection of light-energy by body tissue and/or fluid changes the spectrum of that light-energy and this change in spectrum is analyzed to get information about the composition of that body tissue and/or fluid. In an example, a spectroscopic sensor can comprise a light-energy emitter (e.g. light source) and a light-energy receiver (e.g. photodetector) on the same side (e.g. the ventral or dorsal side) of a body member, wherein the light-energy receiver receives light from the light-energy emitter after that light has been reflected by body tissue and/or fluid. In an example, light-energy from a light-energy emitter can be directed toward body tissue and/or fluid, reflected by the body tissue and/or fluid, and then received by the light-energy receiver. In an example, changes in the spectrum of light which has been reflected by body tissue and/or fluid can be analyzed to estimate the value of a biometric parameter or changes in that value over time.

In an example, a light-energy emitter can deliver light-energy to body tissue and/or fluid via direct optical communication. In an example, a system can further comprise one or more light guides which guide light from a light-energy emitter toward body tissue and/or fluid at a selected angle or location. In an example, a system can further comprise one or more lenses which guide light from a light-energy emitter toward body tissue and/or fluid at a selected angle or location. In an example, a system can further comprise one or more prisms which guide light from a light-energy emitter toward body tissue and/or fluid at a selected angle or location. In an example, a system can further comprise one or more optical filters which modify the spectrum of light directed toward body tissue and/or fluid. In an example, a beam of light can be emitted by a light-energy emitter, pass through a first side of an angled one-way mirror, hit body tissue, reflect back from the body tissue, reflect off a second side of the angled one-way mirror, and then enter a light-energy receiver.

In an example, a light-energy receiver can receive light-energy which has interacted with body tissue and/or fluid via direct optical communication. In an example, a system can further comprise one or more light guides which guide light from body tissue and/or fluid to a light-energy receiver. In an example, a system can further comprise one or more lenses which guide light from body tissue and/or fluid to a light-energy receiver. In an example, a system can further comprise one or more prisms which guide light from body tissue and/or fluid to a light-energy receiver. In an example, a system can further comprise one or more optical filters which modify the spectrum of light from body tissue and/or fluid before it reaches a light-energy receiver.

In an example, a wearable device of this system can be a spectroscopic sensor (including a light-energy emitter and light-energy receiver) which collects light-energy data, wherein this data is analyzed using spectroscopic analysis in order to monitor changes in the chemical composition of body tissue and/or fluid. In an example, changes, gaps, and/or shifts in selected frequencies in the spectrum of transmitted or reflected light due to an interaction with a person's body tissue and/or fluid can be analyzed to estimate the chemical composition of the person's body tissue and/or fluid. In an example, portions of the spectrum of light emitted by a light-energy emitter can be absorbed by body tissue. Spectral analysis of these absorbed portions enables measurement of analyte levels in a person's body.

In an example, a wearable device of this system can comprise a spectroscopic sensor with a light-energy receiver which receives ambient light which has passed through body tissue and/or fluid or has been reflected by body tissue and/or fluid. In an example, changes, gaps, and/or shifts in selected frequencies in the spectrum of ambient light due to interaction with a person's body tissue and/or fluid can be analyzed to monitor changes in the chemical composition of the person's body tissue and/or fluid. In an example, portions of the spectrum of ambient light can be reduced and/or shifted by interaction with body tissue and spectral analysis of these shifted portions can enable measurement of an analyte level in the body.

In an example, a wearable device of this system can have a near-infrared spectroscopic sensor. In an example, a wearable device can have an infrared spectroscopic sensor. In an example, a wearable device of this system can have both a near-infrared spectroscopic sensor and an infrared spectroscopic sensor. In an example, a wearable device can have a spectral analysis sensor. In an example, a wearable device can have a photochemical sensor. In an example, a wearable device can have an ion mobility spectroscopic sensor. In an example, a wearable device can have a backscattering spectrometry sensor.

In an example, a wearable device of this system can have a laser spectroscopic sensor. In an example, a wearable device can have a liquid chromatography sensor. In an example, a wearable device can have a fiber optic spectroscopic sensor. In an example, a wearable device can have an ultraviolet spectroscopic sensor. In an example, a wearable device can have a mass spectrometry sensor. In an example, a wearable device can have a spectrometric sensor. In an example, a wearable device can have a fluorescence sensor. In an example, a wearable device of this system can have a visible or white light spectroscopic sensor. In an example, a wearable device can have a gas chromatography sensor. In an example, a wearable device can have an ambient light spectroscopic sensor. In an example, a wearable device can have a spectrometry sensor. In an example, a wearable device can have a chemiluminescence sensor.

In an example, a wearable device of this system can have a chromatographic sensor. In an example, a wearable device can have a spectroscopic oximeter. In an example, a wearable device can have a colorimetric sensor. In an example, a wearable device can have an ultraviolet light sensor. In an example, a wearable device can have a Raman spectroscopy sensor. In an example, a wearable device can have an analytical chromatographic sensor. In an example, a wearable device can have a spectrophotometer. In an example, a wearable device can have a photocell. In an example, a wearable device can have a coherent light spectroscopic sensor. In an example, a wearable device can have an optoelectronic sensor.

In an example, a closed loop system for human circulatory assistance can comprise: a finger ring, wherein the finger ring further comprises a light-energy emitter and a light-energy receiver, wherein the light-energy receiver receives light-energy from the light-energy emitter after this light-energy has been transmitted through and/or reflected from the person's body tissue and/or blood, wherein changes in the spectrum of the light-energy due to its transmission through and/or reflection from the person's body tissue and/or blood are used to measure a biometric parameter; and an implanted cardiac pacemaker; wherein the cardiac pacemaker is in electromagnetic communication with the person's heart; and wherein one or more of the following cardiac pacemaker functions are triggered when the person has an abnormal biometric parameter value: change in heart electromagnetic stimulation voltage, change in the degree of coordination and/or timing between stimulations to different heart chambers, change in the frequency of heart contraction stimulations, change in the locations on the heart to which electromagnetic energy is delivered, change in the magnitude of heart contraction stimulations, change in the regularity of heart contraction stimulations, delivery of an electromagnetic shock to the heart, and more precise coordination of contraction of different heart chambers.

In an example, a closed loop system for human circulatory assistance can comprise: a finger ring, wherein the finger ring further comprises a light-energy emitter and a light-energy receiver, wherein the light-energy receiver receives light-energy from the light-energy emitter after this light-energy has been transmitted through and/or reflected from the person's body tissue and/or blood, wherein changes in the spectrum of the light-energy due to its transmission through and/or reflection from the person's body tissue and/or blood are used to measure a biometric parameter; and a central (heart-assist) blood pump; wherein one or more of the following central (heart-assist) blood pump functions is triggered when the person has an abnormal biometric parameter value: activation or deactivation of the pump to change blood flow; change in the duration of pump operation to change blood flow; change in the magnitude of pump undulation, compression, or contraction (depending on type of pump) to change blood flow; and change in the speed of a pump's rotation, undulation, compression, or contraction (depending on type of pump) to change blood flow.

In an example, a closed loop system for human circulatory assistance can comprise: a finger ring, wherein the finger ring further comprises a light-energy emitter and a light-energy receiver, wherein the light-energy receiver receives light-energy from the light-energy emitter after this light-energy has been transmitted through and/or reflected from the person's body tissue and/or blood, wherein changes in the spectrum of the light-energy due to its transmission through and/or reflection from the person's body tissue and/or blood are used to measure a biometric parameter; and a non-central (peripheral) blood pump, wherein one or more of the following non-central (peripheral) blood pump functions is triggered when the person has an abnormal biometric parameter value: activation of the pump to increase blood flow; increase in the duration of pump operation to increase blood flow; increase in the magnitude of pump undulation, compression, or contraction (depending on type of pump) to increase blood flow; increase in the speed of a pump's rotation, undulation, compression, or contraction (depending on type of pump) to increase blood flow; and selective activation of a sub-set of non-central (peripheral) blood pumps to change blood flow in a selected sub-set of body locations.

In an example, a closed loop system for human circulatory assistance can comprise: a smart watch or wrist band, wherein the smart watch or wrist band further comprises a light-energy emitter and a light-energy receiver, wherein the light-energy receiver receives light-energy from the light-energy emitter after this light-energy has been transmitted through and/or reflected from the person's body tissue and/or blood, wherein changes in the spectrum of the light-energy due to its transmission through and/or reflection from the person's body tissue and/or blood are used to measure a biometric parameter; and an implanted cardiac pacemaker; wherein the cardiac pacemaker is in electromagnetic communication with the person's heart; and wherein one or more of the following cardiac pacemaker functions are triggered when the person has an abnormal biometric parameter value: change in heart electromagnetic stimulation voltage, change in the degree of coordination and/or timing between stimulations to different heart chambers, change in the frequency of heart contraction stimulations, change in the locations on the heart to which electromagnetic energy is delivered, change in the magnitude of heart contraction stimulations, change in the regularity of heart contraction stimulations, delivery of an electromagnetic shock to the heart, and more precise coordination of contraction of different heart chambers.

In an example, a closed loop system for human circulatory assistance can comprise: a smart watch or wrist band, wherein the smart watch or wrist band further comprises a light-energy emitter and a light-energy receiver, wherein the light-energy receiver receives light-energy from the light-energy emitter after this light-energy has been transmitted through and/or reflected from the person's body tissue and/or blood, wherein changes in the spectrum of the light-energy due to its transmission through and/or reflection from the person's body tissue and/or blood are used to measure a biometric parameter; and a central (heart-assist) blood pump; wherein one or more of the following central (heart-assist) blood pump functions is triggered when the person has an abnormal biometric parameter value: activation or deactivation of the pump to change blood flow; change in the duration of pump operation to change blood flow; change in the magnitude of pump undulation, compression, or contraction (depending on type of pump) to change blood flow; and change in the speed of a pump's rotation, undulation, compression, or contraction (depending on type of pump) to change blood flow.

In an example, a closed loop system for human circulatory assistance can comprise: a smart watch or wrist band, wherein the smart watch or wrist band further comprises a light-energy emitter and a light-energy receiver, wherein the light-energy receiver receives light-energy from the light-energy emitter after this light-energy has been transmitted through and/or reflected from the person's body tissue and/or blood, wherein changes in the spectrum of the light-energy due to its transmission through and/or reflection from the person's body tissue and/or blood are used to measure a biometric parameter; and a non-central (peripheral) blood pump, wherein one or more of the following non-central (peripheral) blood pump functions is triggered when the person has an abnormal biometric parameter value: activation of the pump to increase blood flow; increase in the duration of pump operation to increase blood flow; increase in the magnitude of pump undulation, compression, or contraction (depending on type of pump) to increase blood flow; increase in the speed of a pump's rotation, undulation, compression, or contraction (depending on type of pump) to increase blood flow; and selective activation of a sub-set of non-central (peripheral) blood pumps to change blood flow in a selected sub-set of body locations.

In an example, the biometric parameter which is measured and managed by this system can be selected from the group consisting of: oxygenation level, carbon dioxide level, lactate or lactic acid level, blood pressure, heart rate variability, pulsatile blood volume, pulsatile blood lag, hydration level, respiration rate, exhaled gas composition, body glucose level, troponin level, body motion or exercise level, and sleep status or stage. In an example, a wearable device can collect data on a biometric parameter selected from the group consisting of: oxygenation level, carbon dioxide level, lactate or lactic acid level, blood pressure, heart rate variability, pulsatile blood volume, pulsatile blood lag, hydration level, respiration rate, exhaled gas composition, body glucose level, troponin level, body motion or exercise level, and sleep status or stage. In an example, a finger ring, smart watch, smart watch band, wrist band, ankle band, smart sock, ear ring, ear clip, or ear bud can collect data on a biometric parameter selected from the group consisting of: oxygenation level, carbon dioxide level, lactate or lactic acid level, blood pressure, heart rate variability, pulsatile blood volume, pulsatile blood lag, hydration level, respiration rate, exhaled gas composition, body glucose level, troponin level, body motion or exercise level, and sleep status or stage.

In an example, the operation of an implanted cardiac pacemaker can be controlled and/or adjusted based on a biometric parameter selected from the group consisting of: oxygenation level, carbon dioxide level, lactate or lactic acid level, blood pressure, heart rate variability, pulsatile blood volume, pulsatile blood lag, hydration level, respiration rate, exhaled gas composition, body glucose level, troponin level, body motion or exercise level, and sleep status or stage. In an example, the operation of an implanted central (heart-assist) blood pump can be controlled and/or adjusted based on a biometric parameter selected from the group consisting of: oxygenation level, carbon dioxide level, lactate or lactic acid level, blood pressure, heart rate variability, pulsatile blood volume, pulsatile blood lag, hydration level, respiration rate, exhaled gas composition, body glucose level, troponin level, body motion or exercise level, and sleep status or stage. In an example, the operation of an implanted non-central (peripheral) blood pump can be controlled and/or adjusted based on a biometric parameter selected from the group consisting of: oxygenation level, carbon dioxide level, lactate or lactic acid level, blood pressure, heart rate variability, pulsatile blood volume, pulsatile blood lag, hydration level, respiration rate, exhaled gas composition, body glucose level, troponin level, body motion or exercise level, and sleep status or stage.

In an example, a biometric parameter which is measured and managed by this system can be body oxygenation level or changes in body oxygenation levels. In an example, one or more oxygen-related biometric parameters can be selected from the group consisting of: arterial oxygen saturation level, oxygen metabolism level, saturation of peripheral oxygen, brain oxygenation level, and peripheral tissue oxygenation level. In an example, a wearable device can be a pulse oximeter. In an example, the wearable device can measure blood volume variation over time. In an example, the wearable device can perform photoplethysmography (PPG). In an example, blood oxygen saturation can be based on differential absorption of two different light wavelengths by blood. In an example, operation of an implanted circulatory assistance device can be controlled and/or adjusted based on body oxygenation level or changes in body oxygenation levels. In an example, body oxygenation levels can be measured from multiple locations on a person's body.

In an example, a closed loop system for human circulatory assistance can increase blood circulation by adjusting the operation of a cardiac pacemaker in response to a low body oxygenation level. In an example, a closed loop system can adjust the operation of a cardiac pacemaker in response to low body oxygenation in one or more ways selected from the group consisting of: increase in heart electromagnetic stimulation voltage; increase in the degree of coordination and/or timing between stimulations to different heart chambers; increase in the frequency of heart contraction stimulations; change in the locations on the heart to which electromagnetic energy is delivered; increase in the magnitude of heart contraction stimulations; increase in the regularity of heart contraction stimulations; and more precise coordination of contraction of different heart chambers.

In an example, a closed loop system for human circulatory assistance can comprise: a wearable oximeter, wherein the wearable oximeter uses spectroscopy to measure body oxygenation level; and an implanted central (heart-assist) blood pump, wherein operation of the central (heart-assist) blood pump is adjusted based on body oxygenation level. In an example, a closed loop system for human circulatory assistance can comprise: a wearable oximeter which is worn by a person, wherein the wearable oximeter uses spectroscopy to measure body oxygenation level; and an implanted non-central (peripheral) blood pump, wherein operation of the non-central (peripheral) blood pump is adjusted based on body oxygenation level.

In an example, a closed loop system for human circulatory assistance can increase blood circulation by adjusting the operation of a non-central (peripheral) blood pump. In an example, a closed loop system can adjust the operation of a non-central (peripheral) blood pump in response to low body oxygenation in one or more ways selected from the group consisting of: activation of the pump to increase blood flow; increase in the duration of pump operation to increase blood flow; increase in the magnitude of pump undulation, compression, or contraction (depending on type of pump) to increase blood flow; increase in the speed of a pump's rotation, undulation, compression, or contraction (depending on type of pump) to increase blood flow; and selective activation of a sub-set of non-central (peripheral) blood pumps to change blood flow in a selected sub-set of body locations.

In an example, a closed loop system for human circulatory assistance can comprise: a finger ring, wherein the finger ring further comprises a light-energy emitter and a light-energy receiver, wherein the light-energy receiver receives light-energy from the light-energy emitter after this light-energy has been transmitted through and/or reflected from the person's body tissue and/or blood, wherein changes in the spectrum of the light-energy due to its transmission through and/or reflection from the person's body tissue and/or blood are used to measure body oxygenation level; and an implanted cardiac pacemaker; wherein the cardiac pacemaker is in electromagnetic communication with the person's heart; and wherein one or more of the following cardiac pacemaker functions are triggered when the person has a low body oxygenation level: increase in heart electromagnetic stimulation voltage, increase in the degree of coordination and/or timing between stimulations to different heart chambers, increase in the frequency of heart contraction stimulations, change in the locations on the heart to which electromagnetic energy is delivered, increase in the magnitude of heart contraction stimulations, increase in the regularity of heart contraction stimulations, and more precise coordination of contraction of different heart chambers.

In an example, a closed loop system for human circulatory assistance can comprise: a finger ring, wherein the finger ring further comprises a light-energy emitter and a light-energy receiver, wherein the light-energy receiver receives light-energy from the light-energy emitter after this light-energy has been transmitted through and/or reflected from the person's body tissue and/or blood, wherein changes in the spectrum of the light-energy due to its transmission through and/or reflection from the person's body tissue and/or blood are used to measure body oxygenation level; and a central (heart-assist) blood pump; wherein one or more of the following central (heart-assist) blood pump functions is triggered when the person has a low body oxygenation level: activation of the pump to increase blood flow; increase in the duration of pump operation to increase blood flow; increase in the magnitude of pump undulation, compression, or contraction (depending on type of pump) to increase blood flow; and increase in the speed of a pump's rotation, undulation, compression, or contraction (depending on type of pump) to increase blood flow.

In an example, a closed loop system for human circulatory assistance can comprise: a finger ring, wherein the finger ring further comprises a light-energy emitter and a light-energy receiver, wherein the light-energy receiver receives light-energy from the light-energy emitter after this light-energy has been transmitted through and/or reflected from the person's body tissue and/or blood, wherein changes in the spectrum of the light-energy due to its transmission through and/or reflection from the person's body tissue and/or blood are used to measure body oxygenation level; and a non-central (peripheral) blood pump, wherein one or more of the following non-central (peripheral) blood pump functions is triggered when the person has a low body oxygenation level: activation of the pump to increase blood flow; increase in the duration of pump operation to increase blood flow; increase in the magnitude of pump undulation, compression, or contraction (depending on type of pump) to increase blood flow; increase in the speed of a pump's rotation, undulation, compression, or contraction (depending on type of pump) to increase blood flow; and selective activation of a sub-set of non-central (peripheral) blood pumps to increase blood flow in a selected sub-set of body locations.

In an example, a closed loop system for human circulatory assistance can comprise: a smart watch (or wrist band), wherein the smart watch (or wrist band) further comprises a light-energy emitter and a light-energy receiver, wherein the light-energy receiver receives light-energy from the light-energy emitter after this light-energy has been transmitted through and/or reflected from the person's body tissue and/or blood, wherein changes in the spectrum of the light-energy due to its transmission through and/or reflection from the person's body tissue and/or blood are used to measure body oxygenation level; and an implanted cardiac pacemaker; wherein the cardiac pacemaker is in electromagnetic communication with the person's heart; and wherein one or more of the following cardiac pacemaker functions are triggered when the person has a low body oxygenation level: increase in heart electromagnetic stimulation voltage, increase in the degree of coordination and/or timing between stimulations to different heart chambers, increase in the frequency of heart contraction stimulations, change in the locations on the heart to which electromagnetic energy is delivered, increase in the magnitude of heart contraction stimulations, increase in the regularity of heart contraction stimulations, and more precise coordination of contraction of different heart chambers.

In an example, a closed loop system for human circulatory assistance can comprise: a smart watch (or wrist band), wherein the smart watch (or wrist band) further comprises a light-energy emitter and a light-energy receiver, wherein the light-energy receiver receives light-energy from the light-energy emitter after this light-energy has been transmitted through and/or reflected from the person's body tissue and/or blood, wherein changes in the spectrum of the light-energy due to its transmission through and/or reflection from the person's body tissue and/or blood are used to measure body oxygenation level; and a central (heart-assist) blood pump; wherein one or more of the following central (heart-assist) blood pump functions is triggered when the person has a low body oxygenation level: activation of the pump to increase blood flow; increase in the duration of pump operation to increase blood flow; increase in the magnitude of pump undulation, compression, or contraction (depending on type of pump) to increase blood flow; and increase in the speed of a pump's rotation, undulation, compression, or contraction (depending on type of pump) to increase blood flow.

In an example, a closed loop system for human circulatory assistance can comprise: a smart watch (or wrist band), wherein the smart watch (or wrist band) further comprises a light-energy emitter and a light-energy receiver, wherein the light-energy receiver receives light-energy from the light-energy emitter after this light-energy has been transmitted through and/or reflected from the person's body tissue and/or blood, wherein changes in the spectrum of the light-energy due to its transmission through and/or reflection from the person's body tissue and/or blood are used to measure body oxygenation level; and a non-central (peripheral) blood pump, wherein one or more of the following non-central (peripheral) blood pump functions is triggered when the person has a low body oxygenation level: activation of the pump to increase blood flow; increase in the duration of pump operation to increase blood flow; increase in the magnitude of pump undulation, compression, or contraction (depending on type of pump) to increase blood flow; increase in the speed of a pump's rotation, undulation, compression, or contraction (depending on type of pump) to increase blood flow; and selective activation of a sub-set of non-central (peripheral) blood pumps to increase blood flow in a selected sub-set of body locations.

In an example, a biometric parameter which is measured and managed by this system can be the level of carbon dioxide in a person's body tissue and/or fluid. In an example, a wearable device can have a spectroscopic sensor. In an example, a wearable device can measure blood volume variation over time. In an example, a wearable device can perform photoplethysmography. In an example, body carbon dioxide levels can be measured from multiple locations on a person's body. In an example, the operation of an implanted circulatory assistance device can be controlled and/or adjusted based on the carbon dioxide level in a person's body tissue and/or fluid or changes in that level.

In an example, a closed loop system for human circulatory assistance can increase blood circulation by adjusting the operation of a cardiac pacemaker in response to a high carbon dioxide level in a person's body tissue and/or fluid. In an example, a system can adjust the operation of a cardiac pacemaker in response to a high carbon dioxide level in one or more ways selected from the group consisting of: increase in heart electromagnetic stimulation voltage, increase in the degree of coordination and/or timing between stimulations to different heart chambers, increase in the frequency of heart contraction stimulations, change in the locations on the heart to which electromagnetic energy is delivered, increase in the magnitude of heart contraction stimulations, increase in the regularity of heart contraction stimulations, and more precise coordination of contraction of different heart chambers.

In an example, an implanted circulatory assistance device can be a central (heart-assist) blood pump which assists the heart in pumping blood or a non-central (peripheral) blood pump which assists in pumping blood through a peripheral blood vessel to a selected (peripheral) portion of the body. In an example, a closed loop system can increase blood circulation by adjusting the operation of a central (heart-assist) blood pump or a non-central (peripheral) blood pump. In an example, a closed loop system can adjust the operation of a central (heart-assist) blood pump or a non-central (peripheral) blood pump in response to a high body carbon dioxide level in one or more ways selected from the group consisting of: activation of the pump to increase blood flow; increase in the duration of pump operation to increase blood flow; increase in the magnitude of pump undulation, compression, or contraction (depending on type of pump) to increase blood flow; and increase in the speed of a pump's rotation, undulation, compression, or contraction (depending on type of pump) to increase blood flow.

In an example, a closed loop system for human circulatory assistance can comprise: a finger ring, smart watch, or wrist band which is worn by a person, wherein the finger ring, smart watch, or wrist band collects data concerning carbon dioxide level in the person's body; and an implanted cardiac pacemaker, and wherein operation of the cardiac pacemaker is controlled and/or adjusted based on analysis of the data concerning carbon dioxide level in the person's body. In an example, a closed loop system for human circulatory assistance can comprise: a finger ring, smart watch, or wrist band which is worn by a person, wherein the finger ring, smart watch, or wrist band collects data concerning carbon dioxide level in the person's body; and an implanted central (heart-assist) blood pump, wherein operation of the central (heart-assist) blood pump is controlled and/or adjusted based on analysis of the data concerning carbon dioxide level in the person's body. In an example, a closed loop system for human circulatory assistance can comprise: a finger ring, smart watch, or wrist band which is worn by a person, wherein the finger ring, smart watch, or wrist band collects data concerning carbon dioxide level in the person's body; and an implanted non-central (peripheral) blood pump, wherein operation of the non-central (peripheral) blood pump is controlled and/or adjusted based on analysis of the data concerning carbon dioxide level in the person's body.

In an example, the biometric parameter which is measured and managed by this system can be body lactate and/or lactic acid level. In an example, the wearable device can have a spectroscopic sensor. In an example, the wearable device can measure blood volume variation over time. In an example, the wearable device can perform photoplethysmography (PPG). In an example, body lactate and/or lactic acid level can be measured from multiple locations on the person's body. In an example, operation of an implanted circulatory assistance device can be controlled and/or adjusted based on lactate and/or lactic acid level in a person's body tissue and/or fluid or changes in that level.

In an example, the implanted circulatory assistance device can be a cardiac pacemaker. In an example, a system can increase blood circulation by adjusting the operation of an implanted cardiac pacemaker in response to a high body lactate and/or lactic acid level. In an example, a system can adjust the operation of an implanted cardiac pacemaker in response to a high body lactate and/or lactic acid level in one or more ways selected from the group consisting of: increase in heart electromagnetic stimulation voltage, increase in the degree of coordination and/or timing between stimulations to different heart chambers, increase in the frequency of heart contraction stimulations, change in the locations on the heart to which electromagnetic energy is delivered, increase in the magnitude of heart contraction stimulations, increase in the regularity of heart contraction stimulations, delivery of an electromagnetic shock to the heart, and more precise coordination of contraction of different heart chambers.

In an example, an implanted circulatory assistance device can be a central (heart-assist) blood pump which assists the heart in pumping blood or a non-central (peripheral) blood pump which assists in pumping blood through a vessel to a selected (peripheral) portion of the body. In an example, a closed loop system can increase blood circulation by adjusting the operation of a central (heart-assist) blood pump or a non-central (peripheral) blood pump. In an example, a closed loop system can adjust the operation of a central (heart-assist) blood pump or a non-central (peripheral) blood pump in response to a high body lactate and/or lactic acid level in one or more ways selected from the group consisting of: activation of the pump to increase blood flow; increase in the duration of pump operation to increase blood flow; increase in the magnitude of pump undulation, compression, or contraction (depending on type of pump) to increase blood flow; and increase in the speed of a pump's rotation, undulation, compression, or contraction (depending on type of pump) to increase blood flow.

In an example, a closed loop system for human circulatory assistance can comprise: a finger ring, smart watch, or wrist band which collects data concerning lactate and/or lactic acid level in a person's body tissue and/or fluid; and an implanted cardiac pacemaker whose operation is controlled and/or adjusted based on analysis of the data concerning lactate and/or lactic acid level. In an example, a closed loop system for human circulatory assistance can comprise: a finger ring, smart watch, or wrist band which collects data concerning lactate and/or lactic acid level in a person's body tissue and/or fluid; and an implanted central (heart-assist) blood pump whose operation is controlled and/or adjusted based on analysis of the data concerning lactate and/or lactic acid level. In an example, a closed loop system for human circulatory assistance can comprise: a finger ring, smart watch, or wrist band which collects data concerning lactate and/or lactic acid level in a person's body tissue and/or fluid; and an implanted non-central (peripheral) blood pump whose operation is controlled and/or adjusted based on analysis of the data concerning lactate and/or lactic acid level.

In an example, the biometric parameter which is measured and managed by this system can be blood pressure. In an example, a wearable device can have a spectroscopic sensor which measures a person's blood pressure. In an example, a wearable device can have an electromagnetic energy sensor which measures a person's blood pressure. In an example, blood pressure can be measured from multiple locations on a person's body. In an example, the operation of an implanted circulatory assistance device can be controlled and/or adjusted based on a person's blood pressure or changes in their blood pressure.

In an example, an implanted circulatory assistance device can be a cardiac pacemaker. In an example, a system can adjust the operation of an implanted cardiac pacemaker in response to abnormal blood pressure. In an example, a system can adjust the operation of an implanted cardiac pacemaker in response to abnormal blood pressure in one or more ways selected from the group consisting of: change in heart electromagnetic stimulation voltage, change in the degree of coordination and/or timing between stimulations to different heart chambers, change in the frequency of heart contraction stimulations, change in the locations on the heart to which electromagnetic energy is delivered, change in the magnitude of heart contraction stimulations, change in the regularity of heart contraction stimulations, and more precise coordination of contraction of different heart chambers.

In an example, an implanted circulatory assistance device can be a central (heart-assist) blood pump which assists the heart in pumping blood or a non-central (peripheral) blood pump which assists in pumping blood through a vessel to a selected (peripheral) portion of the body. In an example, a closed loop system can adjust the operation of a central (heart-assist) blood pump or a non-central (peripheral) blood pump in response to abnormal blood pressure. In an example, a closed loop system can adjust the operation of a central (heart-assist) blood pump or a non-central (peripheral) blood pump in response to blood pressure in one or more ways selected from the group consisting of: activation of the device to change blood flow; adjusted device pumping volume to change blood flow; adjusted device rotation and/or speed to change blood flow; changed duration of device operation to change blood flow; selective activation of a sub-set of non-central (peripheral) blood pumps to change blood flow in a selected body location.

In an example, a closed loop system for human circulatory assistance can comprise: a finger ring, smart watch, or wrist band which collects data concerning a person's blood pressure; and an implanted cardiac pacemaker whose operation is controlled and/or adjusted based on analysis of the data concerning the person's blood pressure. In an example, a closed loop system for human circulatory assistance can comprise: a finger ring, smart watch, or wrist band which collects data concerning a person's blood pressure; and an implanted central (heart-assist) blood pump whose operation is controlled and/or adjusted based on analysis of the data concerning the person's blood pressure. In an example, a closed loop system for human circulatory assistance can comprise: a finger ring, smart watch, or wrist band which collects data concerning a person's blood pressure; and an implanted non-central (peripheral) blood pump whose operation is controlled and/or adjusted based on analysis of the data concerning the person's blood pressure.

In an example, a biometric parameter which is measured and managed by this system can be (peripherally measured) Heart Rate Variability (HRV). HRV can be associated with myocardium infarction, congestive cardiac insufficiency, or diabetic neuropathology. In an example, a wearable device can have a spectroscopic sensor. In an example, HRV can be measured from multiple locations on the person's body. In an example, operation of the implanted circulatory assistance device can be controlled and/or adjusted based on HRV or changes in HRV. In an example, a system can monitor for tachycardia or bradycardia.

In an example, an implanted circulatory assistance device of this system can be a cardiac pacemaker. In an example, this system can increase blood circulation by adjusting the operation of a cardiac pacemaker in response to high Heart Rate Variability (HRV). In an example, a closed loop system can adjust the operation of a cardiac pacemaker in response to high Heart Rate Variability (HRV) in one or more ways selected from the group consisting of: change in heart electromagnetic stimulation voltage, change in the degree of coordination and/or timing between stimulations to different heart chambers, change in the frequency of heart contraction stimulations, change in the locations on the heart to which electromagnetic energy is delivered, change in the magnitude of heart contraction stimulations, change in the regularity of heart contraction stimulations, delivery of an electromagnetic shock to the heart, and more precise coordination of contraction of different heart chambers.

In an example, an implanted circulatory assistance device can be a central (heart-assist) blood pump which assists the heart in pumping blood or a non-central (peripheral) blood pump which assists in pumping blood through a vessel to a selected (peripheral) portion of the body. In an example, a closed loop system can increase blood circulation by adjusting the operation of a central (heart-assist) blood pump or a non-central (peripheral) blood pump. In an example, a closed loop system can adjust the operation of a central (heart-assist) blood pump or a non-central (peripheral) blood pump in response to high Heart Rate Variability (HRV) in one or more ways selected from the group consisting of: activation or deactivation of the pump to change blood flow; change in the duration of pump operation to change blood flow; change in the magnitude of pump undulation, compression, or contraction (depending on type of pump) to change blood flow; change in the speed of a pump's rotation, undulation, compression, or contraction (depending on type of pump) to change blood flow; and selective activation of a sub-set of non-central (peripheral) blood pumps to change blood flow in a selected sub-set of body locations.

In an example, the biometric parameter which is measured and monitored in this system can be Pulsatile Blood Volume (PBV) which is measured. In an example, PBV variation can be measured as the percentage change in blood vessel diameter during pulsation. In an example, PBV variation can be measured as the percentage change in absorption of light in a given portion of the light spectrum during pulsation. In an example, the wearable device can have a spectroscopic sensor. In an example, a wearable device can measure blood volume variation over time. In an example, a wearable device can perform photoplethysmography. In an example, Pulsatile Blood Volume (PBV) can be measured from multiple locations on a person's body. In an example, operation of an implanted circulatory assistance device can be controlled and/or adjusted based on Pulsatile Blood Volume (PBV) or variation thereof.

In an example, an implanted circulatory assistance device can be a cardiac pacemaker. In an example, a closed loop system can increase blood circulation by adjusting the operation of a cardiac pacemaker in response to abnormal Pulsatile Blood Volume (PBV) or variation thereof. In an example, a closed loop system can adjust the operation of a cardiac pacemaker in response to abnormal Pulsatile Blood Volume (PBV) in one or more ways selected from the group consisting of: change in heart electromagnetic stimulation voltage, change in the degree of coordination and/or timing between stimulations to different heart chambers, change in the frequency of heart contraction stimulations, change in the locations on the heart to which electromagnetic energy is delivered, change in the magnitude of heart contraction stimulations, change in the regularity of heart contraction stimulations, delivery of an electromagnetic shock to the heart, and more precise coordination of contraction of different heart chambers.

In an example, the implanted circulatory assistance device can be a central (heart-assist) blood pump which assists the heart in pumping blood or a non-central (peripheral) blood pump which assists in pumping blood through a vessel to a selected (peripheral) portion of the body. In an example, a closed loop system can increase blood circulation by adjusting the operation of a central (heart-assist) blood pump or a non-central (peripheral) blood pump. In an example, a system can adjust the operation of a central (heart-assist) blood pump or a non-central (peripheral) blood pump in response to abnormal Pulsatile Blood Volume (PBV), or variation thereof, in one or more ways selected from the group consisting of: activation or deactivation of the pump to change blood flow; change in the duration of pump operation to change blood flow; change in the magnitude of pump undulation, compression, or contraction (depending on type of pump) to change blood flow; change in the speed of a pump's rotation, undulation, compression, or contraction (depending on type of pump) to change blood flow; and selective activation of a sub-set of non-central (peripheral) blood pumps to change blood flow in a selected sub-set of body locations.

In an example, the biometric parameter which is measured and managed by this system can be Pulsatile Blood Lag (PBL). In an example, PBLV can be measured as variation in the lag time between central cardiac pulsation and peripheral blood pulsation. In an example, a wearable device can have a spectroscopic sensor. In an example, the wearable device can measure blood volume variation over time. In an example, the wearable device can perform photoplethysmography. In an example, Pulsatile Blood Lag (PBL) can be measured from multiple locations on the person's body. In an example, operation of the implanted circulatory assistance device can be controlled and/or adjusted based on Pulsatile Blood Lag (PBL) or changes in PBL.

In an example, an implanted circulatory assistance device can be a cardiac pacemaker. In an example, a closed loop system can increase blood circulation by adjusting the operation of a cardiac pacemaker in response to abnormal Pulsatile Blood Lag (PBL). In an example, a closed loop system can adjust the operation of a cardiac pacemaker in response to abnormal Pulsatile Blood Lag (PBL) in one or more ways selected from the group consisting of: change in heart electromagnetic stimulation voltage, change in the degree of coordination and/or timing between stimulations to different heart chambers, change in the frequency of heart contraction stimulations, change in the locations on the heart to which electromagnetic energy is delivered, change in the magnitude of heart contraction stimulations, change in the regularity of heart contraction stimulations, delivery of an electromagnetic shock to the heart, and more precise coordination of contraction of different heart chambers.

In an example, an implanted circulatory assistance device can be a central (heart-assist) blood pump which assists the heart in pumping blood or a non-central (peripheral) blood pump which assists in pumping blood through a vessel to a selected (peripheral) portion of the body. In an example, a closed loop system can increase blood circulation by adjusting the operation of a central (heart-assist) blood pump or a non-central (peripheral) blood pump. In an example, a closed loop system can adjust the operation of a central (heart-assist) blood pump or a non-central (peripheral) blood pump in response to abnormal Pulsatile Blood Lag (PBL) in one or more ways selected from the group consisting of: activation or deactivation of the pump to change blood flow; change in the duration of pump operation to change blood flow; change in the magnitude of pump undulation, compression, or contraction (depending on type of pump) to change blood flow; change in the speed of a pump's rotation, undulation, compression, or contraction (depending on type of pump) to change blood flow; and selective activation of a sub-set of non-central (peripheral) blood pumps to change blood flow in a selected sub-set of body locations.

In an example, the biometric parameter which is measured and managed by this system can be body hydration level. In an example, the wearable device can have a spectroscopic sensor or electromagnetic energy sensor which measures the hydration level of a person's body tissue and/or fluid. In an example, body hydration level can be measured from multiple locations on the person's body. In an example, the operation of the implanted circulatory assistance device can be controlled and/or adjusted based on a person's body hydration level or changes in that level.

In an example, an implanted circulatory assistance device can be an implanted cardiac pacemaker. In an example, a system can change blood circulation by adjusting the operation of a cardiac pacemaker in response to an abnormal body hydration level. In an example, a system can adjust the operation of a cardiac pacemaker in response to an abnormal body hydration level in one or more ways selected from the group consisting of: change in heart electromagnetic stimulation voltage, change in the degree of coordination and/or timing between stimulations to different heart chambers, change in the frequency of heart contraction stimulations, change in the locations on the heart to which electromagnetic energy is delivered, change in the magnitude of heart contraction stimulations, change in the regularity of heart contraction stimulations, delivery of an electromagnetic shock to the heart, and more precise coordination of contraction of different heart chambers.

In an example, an implanted circulatory assistance device can be a central (heart-assist) blood pump which assists the heart in pumping blood or a non-central (peripheral) blood pump which assists in pumping blood through a vessel to a selected (peripheral) portion of the body. In an example, a closed loop system can change blood circulation by adjusting the operation of a central (heart-assist) blood pump or a non-central (peripheral) blood pump. In an example, a closed loop system can adjust the operation of a central (heart-assist) blood pump or a non-central (peripheral) blood pump in response to an abnormal body hydration level in one or more ways selected from the group consisting of: activation of the device to change blood flow; adjusted device pumping volume to change blood flow; adjusted device rotation and/or speed to change blood flow; changed duration of device operation to change blood flow; selective activation of a sub-set of non-central (peripheral) blood pumps to change blood flow in a selected body location.

In an example, the biometric parameter which is measured and managed by this system can be respiration rate. In an example, a wearable device can comprise a motion sensor, spectroscopic sensor, or electromagnetic energy sensor which measures respiration rate. In an example, respiration rate can be measured from multiple locations on the person's body. In an example, the operation of the implanted circulatory assistance device can be controlled and/or adjusted based on respiration rate or changes thereof.

In an example, an implanted circulatory assistance device can be am implanted cardiac pacemaker. In an example, a system can change blood circulation by adjusting the operation of a cardiac pacemaker in response to respiration rate. In an example, a system can adjust the operation of a cardiac pacemaker in response to respiration rate in one or more ways selected from the group consisting of: change in heart electromagnetic stimulation voltage, change in the degree of coordination and/or timing between stimulations to different heart chambers, change in the frequency of heart contraction stimulations, change in the locations on the heart to which electromagnetic energy is delivered, change in the magnitude of heart contraction stimulations, change in the regularity of heart contraction stimulations, delivery of an electromagnetic shock to the heart, and more precise coordination of contraction of different heart chambers.

In an example, an implanted circulatory assistance device can be an implanted central (heart-assist) blood pump which assists the heart in pumping blood or a non-central (peripheral) blood pump which assists in pumping blood through a vessel to a selected (peripheral) portion of the body. In an example, a closed loop system can adjust the operation of a central (heart-assist) blood pump or a non-central (peripheral) blood pump based on a person's respiration rate. In an example, a closed loop system can adjust the operation of a central (heart-assist) blood pump or a non-central (peripheral) blood pump in response to respiration rate in one or more ways selected from the group consisting of: activation of the device to change blood flow; adjusted device pumping volume to change blood flow; adjusted device rotation and/or speed to change blood flow; changed duration of device operation to change blood flow; selective activation of a sub-set of non-central (peripheral) blood pumps to change blood flow in a selected body location.

In an example, the biometric parameter which is measured and managed by this system can be exhaled gas composition (i.e. the composition of gas exhaled by a person). In an example, a wearable device can have a spectroscopic sensor or electromagnetic energy sensor which measures exhaled gas composition. In an example, exhaled gas composition can be measured by a nose ring or eyeglasses bridge. In an example, the operation of the implanted circulatory assistance device can be controlled and/or adjusted based on a person's exhaled gas composition or changes thereof.

In an example, an implanted circulatory assistance device can be am implanted cardiac pacemaker. In an example, a system can change blood circulation by adjusting the operation of a cardiac pacemaker in response to exhaled gas composition. In an example, a system can adjust the operation of a cardiac pacemaker in response to exhaled gas composition in one or more ways selected from the group consisting of: change in heart electromagnetic stimulation voltage, change in the degree of coordination and/or timing between stimulations to different heart chambers, change in the frequency of heart contraction stimulations, change in the locations on the heart to which electromagnetic energy is delivered, change in the magnitude of heart contraction stimulations, change in the regularity of heart contraction stimulations, delivery of an electromagnetic shock to the heart, and more precise coordination of contraction of different heart chambers.

In an example, an implanted circulatory assistance device can be an implanted central (heart-assist) blood pump which assists the heart in pumping blood or a non-central (peripheral) blood pump which assists in pumping blood through a vessel to a selected (peripheral) portion of the body. In an example, a closed loop system can adjust the operation of a central (heart-assist) blood pump or a non-central (peripheral) blood pump based on a person's exhaled gas composition. In an example, a closed loop system can adjust the operation of a central (heart-assist) blood pump or a non-central (peripheral) blood pump in response to exhaled gas composition in one or more ways selected from the group consisting of: activation of the device to change blood flow; adjusted device pumping volume to change blood flow; adjusted device rotation and/or speed to change blood flow; changed duration of device operation to change blood flow; selective activation of a sub-set of non-central (peripheral) blood pumps to change blood flow in a selected body location.

In an example, the biometric parameter which is measured and managed by this system can be the glucose level in a person's body tissue and/or fluid. In an example, the biometric parameter can be blood glucose level. In an example, a wearable device can have a spectroscopy sensor or electromagnetic energy sensor which measures the glucose level of a person's body tissue and/or fluid. In an example, body glucose level can be measured from multiple locations on a person's body. In an example, operation of the implanted circulatory assistance device can be controlled and/or adjusted based on body glucose level or changes thereof.

In an example, an implanted circulatory assistance device can be a cardiac pacemaker. In an example, a system can change the operation of a cardiac pacemaker in response to an abnormal body glucose level. In an example, a system can adjust the operation of a cardiac pacemaker in response to an abnormal body glucose level in one or more ways selected from the group consisting of: change in heart electromagnetic stimulation voltage, change in the degree of coordination and/or timing between stimulations to different heart chambers, change in the frequency of heart contraction stimulations, change in the locations on the heart to which electromagnetic energy is delivered, change in the magnitude of heart contraction stimulations, change in the regularity of heart contraction stimulations, delivery of an electromagnetic shock to the heart, and more precise coordination of contraction of different heart chambers.

In an example, the implanted circulatory assistance device can be a central (heart-assist) blood pump which assists the heart in pumping blood or a non-central (peripheral) blood pump which assists in pumping blood through a vessel to a selected (peripheral) portion of the body. In an example, a closed loop system can adjust the operation of a central (heart-assist) blood pump or a non-central (peripheral) blood pump in response to an abnormal body glucose level. In an example, a closed loop system can adjust the operation of a central (heart-assist) blood pump or a non-central (peripheral) blood pump in response to abnormal body glucose level in one or more ways selected from the group consisting of: activation of the device to change blood flow; adjusted device pumping volume to change blood flow; adjusted device rotation and/or speed to change blood flow; changed duration of device operation to change blood flow; and selective activation of a sub-set of non-central (peripheral) blood pumps to change blood flow in a selected body location.

In an example, the biometric parameter which is measured and managed by this system can be troponin level. In an example, the wearable device can have a spectroscopic sensor or electromagnetic energy sensor which measures the level of troponin in a person's body tissue and/or fluid. In an example, troponin level can be measured from multiple locations on the person's body. In an example, the operation of the implanted circulatory assistance device can be controlled and/or adjusted based on troponin level or a change thereof.

In an example, an implanted circulatory assistance device can be a cardiac pacemaker or ICD. In an example, a system can change blood circulation by adjusting the operation of a cardiac pacemaker or ICD in response to troponin. In an example, a system can adjust the operation of a cardiac pacemaker or ICD in response to troponin in one or more ways selected from the group consisting of: change in heart electromagnetic stimulation voltage, change in the degree of coordination and/or timing between stimulations to different heart chambers, change in the frequency of heart contraction stimulations, change in the locations on the heart to which electromagnetic energy is delivered, change in the magnitude of heart contraction stimulations, change in the regularity of heart contraction stimulations, delivery of an electromagnetic shock to the heart, and more precise coordination of contraction of different heart chambers.

In an example, the implanted circulatory assistance device can be a central (heart-assist) blood pump which assists the heart in pumping blood or a non-central (peripheral) blood pump which assists in pumping blood through a vessel to a selected (peripheral) portion of the body. In an example, a closed loop system can change blood circulation by adjusting the operation of a central (heart-assist) blood pump or a non-central (peripheral) blood pump. In an example, a closed loop system can adjust the operation of a central (heart-assist) blood pump or a non-central (peripheral) blood pump in response to troponin in one or more ways selected from the group consisting of: activation of the device to change blood flow; adjusted device pumping volume to change blood flow; adjusted device rotation and/or speed to change blood flow; changed duration of device operation to change blood flow; selective activation of a sub-set of non-central (peripheral) blood pumps to change blood flow in a selected body location.

In an example, the biometric parameter which is measured and managed by this system can be body motion or exercise level. In an example, the wearable device can have a motion sensor, GPS sensor, or EMG sensor which measures body motion and/or exercise level. In an example, body motion or exercise level can be measured from multiple locations on the person's body. In an example, the operation of an implanted circulatory assistance device can be controlled and/or adjusted based on body motion or exercise level or changes thereof.

In an example, an implanted circulatory assistance device can be a cardiac pacemaker. In an example, a system can change blood circulation by adjusting the operation of a cardiac pacemaker in response to a high body motion or exercise level. In an example, a system can adjust the operation of a cardiac pacemaker in response to a high body motion or exercise level in one or more ways selected from the group consisting of: increase in heart electromagnetic stimulation voltage, increase in the degree of coordination and/or timing between stimulations to different heart chambers, increase in the frequency of heart contraction stimulations, change in the locations on the heart to which electromagnetic energy is delivered, increase in the magnitude of heart contraction stimulations, increase in the regularity of heart contraction stimulations, delivery of an electromagnetic shock to the heart, and more precise coordination of contraction of different heart chambers.

In an example, an implanted circulatory assistance device can be a central (heart-assist) blood pump which assists the heart in pumping blood or a non-central (peripheral) blood pump which assists in pumping blood through a vessel to a selected (peripheral) portion of the body. In an example, a closed loop system can adjust the operation of a central (heart-assist) blood pump or a non-central (peripheral) blood pump in response to body motion or exercise level. In an example, a closed loop system can adjust the operation of a central (heart-assist) blood pump or a non-central (peripheral) blood pump in response to a high body motion or exercise level in one or more ways selected from the group consisting of: activation of the device to change blood flow; adjusted device pumping volume to change blood flow; adjusted device rotation and/or speed to change blood flow; changed duration of device operation to change blood flow; selective activation of a sub-set of non-central (peripheral) blood pumps to change blood flow in a selected body location.

In an example, a system can adjust a person's cardiac function based on their whole-body posture and/or configuration. In an example, a system can adjust a person's cardiac function based on identification of a specific whole-body posture and/or configuration. In an example, a system can adjust a person's cardiac function based on identification of a specific type of activity based on measured whole-body posture and/or configuration. In an example, a system can increase (or decrease) the frequency of a person's heart beats and/or the magnitude of a person's heart contractions in response to a change in the person's whole-body posture and/or configuration as detected by one or more wearable biometric sensors. In an example, a person's whole-body posture and/or configuration can be measured by one or more motion sensors, electromyographic (EMG sensors), and/or bend sensors.

In an example, the biometric parameter which is measured and monitored by this system can be sleep status or stage. In an example, the wearable device can have a motion sensor or EEG sensor which measures sleep status or stage. In an example, sleep status or stage can be measured from multiple locations on the person's body. In an example, the operation of an implanted circulatory assistance device can be controlled and/or adjusted based on sleep status or stage or change thereof.

In an example, an implanted circulatory assistance device can be an implanted cardiac pacemaker. In an example, a system can change blood circulation by adjusting the operation of a cardiac pacemaker in response to sleep status or stage. In an example, a system can adjust the operation of a cardiac pacemaker in response to sleep status or stage in one or more ways selected from the group consisting of: change in heart electromagnetic stimulation voltage, change in the degree of coordination and/or timing between stimulations to different heart chambers, change in the frequency of heart contraction stimulations, change in the locations on the heart to which electromagnetic energy is delivered, change in the magnitude of heart contraction stimulations, change in the regularity of heart contraction stimulations, delivery of an electromagnetic shock to the heart, and more precise coordination of contraction of different heart chambers.

In an example, an implanted circulatory assistance device can be a central (heart-assist) blood pump which assists the heart in pumping blood or a non-central (peripheral) blood pump which assists in pumping blood through a vessel to a selected (peripheral) portion of the body. In an example, a closed loop system can adjust the operation of a central (heart-assist) blood pump or a non-central (peripheral) blood pump in response to sleep status or stage. In an example, a closed loop system can adjust the operation of a central (heart-assist) blood pump or a non-central (peripheral) blood pump in response to sleep status or stage in one or more ways selected from the group consisting of: activation of the device to change blood flow; adjusted device pumping volume to change blood flow; adjusted device rotation and/or speed to change blood flow; changed duration of device operation to change blood flow; selective activation of a sub-set of non-central (peripheral) blood pumps to change blood flow in a selected body location.

In an example, a closed loop system for human circulatory assistance can comprise: a finger ring, smart watch, or wrist band which collects data concerning sleep status or stage; and an implanted cardiac pacemaker whose operation is controlled and/or adjusted based on analysis of the data concerning sleep status or stage. In an example, a closed loop system for human circulatory assistance can comprise: a finger ring, smart watch, or wrist band which collects data concerning sleep status or stage; and an implanted central (heart-assist) blood pump whose operation is controlled and/or adjusted based on analysis of the data concerning sleep status or stage. In an example, a closed loop system for human circulatory assistance can comprise: a finger ring, smart watch, or wrist band which collects data concerning sleep status or stage; and an implanted non-central (peripheral) blood pump whose operation is controlled and/or adjusted based on analysis of the data concerning sleep status or stage.

In an example, a closed loop system for human circulatory assistance can comprise a plurality of wearable devices which are worn on different locations of a person's body so as to measure values of a biometric parameter from different locations on the person's body. In an example, the operation of an implanted circulatory assistance device can be controlled and/or adjusted by data concerning biometric parameter levels from a plurality of wearable devices on different locations of a person's body. In an example, the operation of an implanted circulatory assistance device can be adjusted based on the average of biometric parameter levels, the lowest biometric parameter level, the highest biometric parameter level, and/or the range or variability of biometric parameter levels measured by a plurality of wearable devices worn at different locations on a person's body. In an example, the operation of an implanted circulatory assistance device can be adjusted based on multivariate analysis of data concerning biometric parameter levels from a plurality of wearable devices worn at different locations on a person's body.

In an example, the operation of an implanted cardiac pacemaker can be controlled and/or adjusted by data concerning biometric parameter levels from a plurality of wearable devices on different locations of a person's body. In an example, the operation of an implanted cardiac pacemaker can be adjusted based on the average of biometric parameter levels, the lowest biometric parameter level, the highest biometric parameter level, and/or the range or variability of biometric parameter levels measured by a plurality of wearable devices worn at different locations on a person's body. In an example, the operation of an implanted cardiac pacemaker can be adjusted based on multivariate analysis of data concerning biometric parameter levels from a plurality of wearable devices worn at different locations on a person's body.

In an example, a closed loop system for human circulatory assistance can comprise: a first wearable device which is worn by a person on a first external location of the person's body, wherein the first wearable device collects data concerning a biometric parameter from the first external location; a second wearable device which is worn by a person on a second external location of the person's body, wherein the second wearable device collects data on the biometric parameter from the second external location; and an implanted cardiac pacemaker, wherein operation of the cardiac pacemaker is adjusted based a difference between the value of the biometric parameter as measured from the first external location and the value of the biometric parameter as measured from the second location.

In an example, a closed loop system for human circulatory assistance can comprise: a first wearable device which is worn by a person on a first external location of the person's body, wherein the first wearable device collects data concerning body oxygenation level from the first external location; a second wearable device which is worn by a person on a second external location of the person's body, wherein the second wearable device collects data concerning body oxygenation level from the second external location; and an implanted cardiac pacemaker, wherein operation of the cardiac pacemaker is adjusted based a difference between the value of body oxygenation level as measured from the first external location and the value of body oxygenation level as measured from the second location.

In an example, the operation of an implanted central (heart-assist) blood pump can be controlled and/or adjusted by data concerning biometric parameter levels from a plurality of wearable devices on different locations of a person's body. In an example, the operation of an implanted central (heart-assist) blood pump can be adjusted based on the average of biometric parameter levels, the lowest biometric parameter level, the highest biometric parameter level, and/or the range or variability of biometric parameter levels measured by a plurality of wearable devices worn at different locations on a person's body. In an example, the operation of an implanted central (heart-assist) blood pump can be adjusted based on multivariate analysis of data concerning biometric parameter levels from a plurality of wearable devices worn at different locations on a person's body.

In an example, a closed loop system for human circulatory assistance can comprise: a first wearable device which is worn by a person on a first external location of the person's body, wherein the first wearable device collects data concerning a biometric parameter from the first external location; a second wearable device which is worn by a person on a second external location of the person's body, wherein the second wearable device collects data on the biometric parameter from the second external location; and an implanted central (heart-assist) blood pump, wherein operation of the central (heart-assist) blood pump is adjusted based a difference between the value of the biometric parameter as measured from the first external location and the value of the biometric parameter as measured from the second location.

In an example, a closed loop system for human circulatory assistance can comprise: a first wearable device which is worn by a person on a first external location of the person's body, wherein the first wearable device collects data concerning body oxygenation level from the first external location; a second wearable device which is worn by a person on a second external location of the person's body, wherein the second wearable device collects data concerning body oxygenation level from the second external location; and an central (heart-assist) blood pump, wherein operation of the central (heart-assist) blood pump is adjusted based a difference between the value of body oxygenation level as measured from the first external location and the value of body oxygenation level as measured from the second location.

In an example, the operation of an implanted non-central (peripheral) blood pump can be controlled and/or adjusted by data concerning biometric parameter levels from a plurality of wearable devices on different locations of a person's body. In an example, the operation of an implanted non-central (peripheral) blood pump can be adjusted based on the average of biometric parameter levels, the lowest biometric parameter level, the highest biometric parameter level, and/or the range or variability of biometric parameter levels measured by a plurality of wearable devices worn at different locations on a person's body. In an example, the operation of an implanted non-central (peripheral) blood pump can be adjusted based on multivariate analysis of data concerning biometric parameter levels from a plurality of wearable devices worn at different locations on a person's body. In an example, the operation of a plurality of implanted non-central (peripheral) blood pumps can be controlled and/or adjusted by data concerning biometric parameter levels from a plurality of wearable devices on different locations of a person's body.

There are many potential advantages of having a plurality of individually-controlled implanted blood pumps distributed throughout a person's body, wherein these blood pumps are adjusted (in a feedback loop) based on biometric measurements from associated external wearable devices. One potential advantage is greater accuracy and selectivity in maintaining biometric parameters (such as oxygenation) in different portions of a person's body. For example, diabetics often suffer from poor blood circulation in their feet and hands. This can lead to wounds which do not heal and even amputation. A system for selective circulatory assistance with a plurality of individually-controllable implanted blood pumps whose operation is adjusted (in real time) based on associated wearable oxygenation sensors can help to avoid this. This can be a significant improvement over a single central cardiac pacemaker or single central (heart-assist) blood pump which whose operation is not informed by the actual oxygen levels in a person's feet and hands.

Although the analogy is not perfect, a closed loop system for circulatory assistance with a plurality of implanted blood pumps whose operations are selectively controlled and/or adjusted by a plurality of wearable biometric sensors is analogous to having a climate control system for a home or other building with different HVAC (e.g. heating or cooling) zones in different areas throughout the home or other building. Having different HVAC zones in different areas allows more accurate and more selective control of temperatures in different areas. Such a system can reduce hot spots or cold spots in a home or building. By analogy, a person's body can have regions with high and low blood circulation (and body oxygenation). A system for circulatory assistance with a plurality of implanted blood pumps can help to maintain proper and consistent blood circulation (and body oxygenation) in all regions of the body. This can help to heal wounds or even perhaps avoid amputations. Having different HVAC zones can also improve the energy efficiency of a building's climate control system. By analogy, having a decentralized plurality of implanted blood pumps can be more energy efficient. Energy efficiency can be a serious consideration in view of the potentially-high energy demands of implanted blood pumps.

In an example, a closed loop system for human circulatory assistance can comprise: a first wearable device which is worn by a person on a first external location of the person's body, wherein the first wearable device collects data concerning a biometric parameter from the first external location; a second wearable device which is worn by a person on a second external location of the person's body, wherein the second wearable device collects data on the biometric parameter from the second external location; a first implanted non-central (peripheral) blood pump, wherein the first implanted non-central (peripheral) blood pump selectively increases blood flow to the first external location of the person's body based on the value of the biometric parameter at the first external location; and a second implanted non-central (peripheral) blood pump, wherein the second implanted non-central (peripheral) blood pump selectively increases blood flow to the second external location of the person's body based on the value of the biometric parameter at the second external location.

In an example, a closed loop system for human circulatory assistance can comprise: a plurality of wearable devices which are worn by a person on different external locations of the person's body, wherein the wearable devices collect data on a biometric parameter from the different external locations; and a plurality of implanted blood pumps which are implanted in different internal locations within the person's body, wherein the implanted circulatory assistance devices provide localized blood circulation assistance from the different internal locations, wherein internal locations and external locations are associated with each other, wherein operation of an implanted blood pump at a selected internal location is adjusted based on analysis of data on the biometric parameter collected by a wearable device at the external location which is paired with that selected internal location.

In an example, a closed loop system for human circulatory assistance can comprise: a first wearable device which is worn by a person on a first external location of the person's body, wherein the first wearable device collects data concerning a biometric parameter from the first external location; a second wearable device which is worn by a person on a second external location of the person's body, wherein the second wearable device collects data concerning a biometric parameter from the second external location; a first implanted blood pump which increases blood flow to the first external location of the person's body; a second implanted blood pump which increases blood flow to the second external location of the person's body; wherein operation of the first implanted blood pump is adjusted based on analysis of data from the first wearable device; and wherein operation of the second implanted blood pump is adjusted based on analysis of data from the second wearable device.

In an example, there are some situations in which it may be desirable to temporarily decrease blood flow to a one body region in order to improve blood flow to another body region where blood flow is more critically needed at the moment. In an example, a closed loop system for human circulatory assistance can comprise: a first wearable device which is worn by a person on a first external location of the person's body, wherein the first wearable device collects data concerning a biometric parameter from the first external location; a second wearable device which is worn by a person on a second external location of the person's body, wherein the second wearable device collects data concerning a biometric parameter from the second external location; a first implanted blood pump which increases blood flow to the first external location of the person's body; a second implanted blood pump which increases blood flow to the second external location of the person's body; wherein operation of the first implanted blood pump is adjusted based on analysis of data from the second wearable device; and wherein operation of the second implanted blood pump is adjusted based on analysis of data from the first wearable device.

In an example, a closed loop system for human circulatory assistance can comprise: a first wearable device which is worn by a person on a first external location of the person's body, wherein the first wearable device measures the value of a biometric parameter from this first location, a second wearable device which is worn by a person on a second external location of the person's body, wherein the second wearable device measures the value of the biometric parameter from this second location; a first implanted blood pump which selectively increases blood flow to the first external location of the person's body, wherein the operation of the first implanted blood pump is selectively activated and/or adjusted when the value of the biometric parameter from the first location is abnormal; and a second implanted blood pump which selectively increases blood flow to the second external location of the person's body, wherein the operation of the second implanted blood pump is selectively activated and/or adjusted when the value of the biometric parameter from the second location is abnormal.

In an example, the operation of an implanted blood pump in a given location can be selectively activated and/or adjusted when the value of a biometric parameter measured from that location is abnormal for at least a given length of time, wherein this length of time is selected from within a range of 10 seconds to 10 minutes. In an example, this length of time can be selected from within a range of 5 minutes to 1 hour. In an example, the value of a biometric parameter can be considered abnormal when it is at least X % lower than the minimum value in a benchmark range of values, wherein X % is selected from within a range of 10% to 50%. In an example, X % can be selected from within a range of 25% to 100%. In an example, the value of a biometric parameter can be considered abnormal when it is at least Y % higher than the maximum value in a benchmark range of values, wherein Y % is selected from within a range of 10% to 50%. In an example, Y % can be selected from within a range of 25% to 100%.

In an example, a closed loop system for human circulatory assistance can comprise: a first wearable device which is worn by a person on a first external location of the person's body, wherein the first wearable device measures the value of a biometric parameter from the first location, a second wearable device which is worn by a person on a second external location of the person's body, wherein the second wearable device measures the value of the biometric parameter from the second location; a first implanted blood pump which selectively increases blood flow to the first external location of the person's body, wherein the first implanted blood pump is selectively activated and/or the operation of the first implanted blood pump is adjusted when the value of the biometric parameter from the first location is X % lower or Y % higher than a benchmark range of values; and a second implanted blood pump which selectively increases blood flow to the second external location of the person's body, wherein the second implanted blood pump is selectively activated and/or the operation of the second implanted blood pump is adjusted when the value of the biometric parameter from the second location is X % lower or Y % higher than a benchmark range of values. In an example, X % can be a percentage selected from within a range of 10% to 50%. In an example, Y % can be a percentage selected from within a range of 10% to 50%.

In an example, the operation of an implanted blood pump in a first location can be selectively activated and/or adjusted based on comparison of the value of a biometric parameter measured from that first location relative to the value of the biometric parameter measured from a second location. In an example, an implanted blood pump in a first location can be selectively activated and/or adjusted when the value of a biometric parameter measured from that first location is at least X % lower or Y % higher than the value of the biometric parameter measured from a second location. In an example, X % can be a percentage selected from within a range of 10% to 50%. In an example, Y % can be a percentage selected from within a range of 10% to 50%. In an example, implanted blood pumps in a plurality of internal locations can be selectively activated and/or adjusted based on multivariate analysis of values of a biometric parameter measured from a plurality of external locations. In an example, a plurality of implanted blood pumps in different locations in a person's vasculature can comprise a distributed network of circulation assisting devices for maintenance of proper blood circulation throughout different body regions.

In an example, a closed loop system for human circulatory assistance can comprise: a first wearable device which is worn by a person on a first external location of the person's body, wherein the first wearable device measures the value of a biometric parameter from this first location, a second wearable device which is worn by a person on a second external location of the person's body, wherein the second wearable device measures the value of a biometric a first implanted blood pump which selectively increases blood flow to the first external location of the person's body, wherein the first implanted blood pump is selectively activated and/or the operation of the first implanted blood pump is adjusted when the value of the biometric parameter from the first location is at least X % lower or Y % higher than the value of the biometric parameter from the second location; and a second implanted blood pump which selectively increases blood flow to the second external location of the person's body, wherein the second implanted blood pump is selectively activated and/or the operation of the second implanted blood pump is adjusted when the value of the biometric parameter from the second location is at least X % lower or Y % higher than the value of the biometric parameter from the first location.

In an example, there are cases when it can be desirable to change blood flow to a first body region based on measurement of a biometric parameter from a second body region. For example, to the extent that there is a short-term constraint on overall blood flow throughout a person's body, it can be desirable to increase relative blood flow in a first body region (when it has inadequate blood flow) if there is adequate blood flow in a second body region. In an example, a closed loop system for human circulatory assistance can comprise: a first wearable device on a first external body location which collects data concerning a biometric parameter from the first external location; a second wearable device on a second external body location which collects data on the biometric parameter from the second external location; a first implanted blood pump which selectively increases blood flow to the first external location of the person's body; and a second implanted blood pump which selectively increases blood flow to the second external location of the person's body; wherein operation of the second implanted blood pump is adjusted based on analysis of data from the first wearable device.

In an example, a closed loop system for human circulatory assistance can comprise: a first wearable device which is worn by a person on a first external location of the person's body, wherein the first wearable device measures the value of body oxygenation level from this first location, a second wearable device which is worn by a person on a second external location of the person's body, wherein the second wearable device measures the value of the body oxygenation level from this second location; a first implanted blood pump which selectively increases blood flow to the first external location of the person's body, wherein the operation of the first implanted blood pump is selectively activated and/or adjusted when the value of the body oxygenation level from the first location is abnormal; and a second implanted blood pump which selectively increases blood flow to the second external location of the person's body, wherein the operation of the second implanted blood pump is selectively activated and/or adjusted when the value of the body oxygenation level from the second location is abnormal.

In an example, the operation of an implanted blood pump in a given location can be selectively activated and/or adjusted when the value of body oxygenation level measured from that location is abnormal for at least a given length of time, wherein this length of time is selected from within a range of 10 seconds to 10 minutes. In an example, this length of time can be selected from within a range of 5 minutes to 1 hour. In an example, the value of body oxygenation level can be considered abnormal when it is at least X % lower than the minimum value in a benchmark range of values, wherein X % is selected from within a range of 10% to 50%. In an example, X % can be selected from within a range of 25% to 100%.

In an example, a closed loop system for human circulatory assistance can comprise: a first wearable device which is worn by a person on a first external location of the person's body, wherein the first wearable device measures the value of body oxygenation level from the first location, a second wearable device which is worn by a person on a second external location of the person's body, wherein the second wearable device measures the value of the body oxygenation level from the second location; a first implanted blood pump which selectively increases blood flow to the first external location of the person's body, wherein the first implanted blood pump is selectively activated and/or the operation of the first implanted blood pump is adjusted when the value of the body oxygenation level from the first location is X % lower than a benchmark range of values; and a second implanted blood pump which selectively increases blood flow to the second external location of the person's body, wherein the second implanted blood pump is selectively activated and/or the operation of the second implanted blood pump is adjusted when the value of the body oxygenation level from the second location is X % lower than a benchmark range of values. In an example, X % can be a percentage selected from within a range of 10% to 50%.

In an example, a closed loop system for human circulatory assistance can comprise: a first wearable device which is worn by a person on a first external location of the person's body, wherein the first wearable device measures the value of body oxygenation level from this first location, a second wearable device which is worn by a person on a second external location of the person's body, wherein the second wearable device measures the value of a biometric a first implanted blood pump which selectively increases blood flow to the first external location of the person's body, wherein the first implanted blood pump is selectively activated and/or the operation of the first implanted blood pump is adjusted when the value of the body oxygenation level from the first location is at least X % lower than the value of the body oxygenation level from the second location; and a second implanted blood pump which selectively increases blood flow to the second external location of the person's body, wherein the second implanted blood pump is selectively activated and/or the operation of the second implanted blood pump is adjusted when the value of the body oxygenation level from the second location is at least X % lower than the value of the body oxygenation level from the first location.

In an example, there are cases when it can be desirable to change blood flow to a first body region based on measurement of body oxygenation level from a second body region. For example, to the extent that there is a short-term constraint on overall blood flow throughout a person's body, it can be desirable to increase relative blood flow in a first body region (when it has inadequate oxygenation) if there is adequate oxygenation in a second body region. In an example, a closed loop system for human circulatory assistance can comprise: a first wearable device on a first external body location which collects data concerning body oxygenation level from the first external location; a second wearable device on a second external body location which collects data concerning body oxygenation level from the second external location; a first implanted blood pump which selectively increases blood flow to the first external location of the person's body; and a second implanted blood pump which selectively increases blood flow to the second external location of the person's body; wherein operation of the second implanted blood pump is adjusted based on analysis of data from the first wearable device.

In an example, a closed loop system for human circulatory assistance can comprise: a first wearable device which is worn by a person on a first external location of the person's body, wherein the first wearable device measures the value of blood pressure from this first location, a second wearable device which is worn by a person on a second external location of the person's body, wherein the second wearable device measures the value of blood pressure from this second location; a first implanted blood pump which selectively increases blood flow to the first external location of the person's body, wherein the operation of the first implanted blood pump is selectively adjusted when the value of blood pressure from the first location is abnormal; and a second implanted blood pump which selectively increases blood flow to the second external location of the person's body, wherein the operation of the second implanted blood pump is selectively adjusted when the value of blood pressure from the second location is abnormal.

In an example, the operation of an implanted blood pump in a given location can be selectively adjusted when the value of blood pressure measured from that location is abnormal for at least a given length of time, wherein this length of time is selected from within a range of 10 seconds to 10 minutes. In an example, this length of time can be selected from within a range of 5 minutes to 1 hour. In an example, the value of blood pressure can be considered abnormal when it is at least X % lower than the minimum value in a benchmark range of values, wherein X % is selected from within a range of 5% to 25%. In an example, X % can be selected from within a range of 20% to 50%. In an example, the value of blood pressure can be considered abnormal when it is at least Y % higher than the maximum value in a benchmark range of values, wherein Y % is selected from within a range of 5% to 25%. In an example, Y % can be selected from within a range of 20% to 50%.

In an example, a closed loop system for human circulatory assistance can comprise: a first wearable device which is worn by a person on a first external location of the person's body, wherein the first wearable device measures the value of blood pressure from the first location, a second wearable device which is worn by a person on a second external location of the person's body, wherein the second wearable device measures the value of blood pressure from the second location; a first implanted blood pump which selectively increases blood flow to the first external location of the person's body, wherein the first implanted blood pump is selectively activated and/or the operation of the first implanted blood pump is adjusted when the value of blood pressure from the first location is X % lower or Y % higher than a benchmark range of values; and a second implanted blood pump which selectively increases blood flow to the second external location of the person's body, wherein the second implanted blood pump is selectively activated and/or the operation of the second implanted blood pump is adjusted when the value of blood pressure from the second location is X % lower or Y % higher than a benchmark range of values. In an example, X % can be a percentage selected from within a range of 10% to 50%. In an example, Y % can be a percentage selected from within a range of 10% to 50%.

In an example, the operation of an implanted blood pump in a first location can be selectively adjusted based on comparison of the value of blood pressure measured from that first location relative to the value of blood pressure measured from a second location. In an example, an implanted blood pump in a first location can be selectively adjusted when the value of blood pressure measured from that first location is at least X % lower or Y % higher than the value of blood pressure measured from a second location. In an example, X % can be a percentage selected from within a range of 10% to 50%. In an example, Y % can be a percentage selected from within a range of 10% to 50%. In an example, implanted blood pumps in a plurality of internal locations can be selectively adjusted based on multivariate analysis of values of blood pressure measured from a plurality of external locations. In an example, a plurality of implanted blood pumps in different locations in a person's vasculature can comprise a distributed network of circulation assisting devices for maintenance of proper blood circulation throughout different body regions.

In an example, a closed loop system for human circulatory assistance can comprise: a first wearable device which is worn by a person on a first external location of the person's body, wherein the first wearable device measures the value of blood pressure from this first location, a second wearable device which is worn by a person on a second external location of the person's body, wherein the second wearable device measures the value of a biometric a first implanted blood pump which selectively increases blood flow to the first external location of the person's body, wherein the first implanted blood pump is selectively activated and/or the operation of the first implanted blood pump is adjusted when the value of blood pressure from the first location is at least X % lower or Y % higher than the value of blood pressure from the second location; and a second implanted blood pump which selectively increases blood flow to the second external location of the person's body, wherein the second implanted blood pump is selectively activated and/or the operation of the second implanted blood pump is adjusted when the value of blood pressure from the second location is at least X % lower or Y % higher than the value of blood pressure from the first location.

In an example, a rapid loss in blood pressure in a first body region relative to a second body region may indicate hemorrhaging in the first body region. In an example, the operation of a non-central (peripheral) blood pump which increases blood flow to a first body region can be adjusted based on a rapid loss in blood pressure as measured by a wearable device on an external location of that first body region. In an example, the operation of a non-central (peripheral) blood pump which controls blood flow to a first body region can be adjusted to reduce blood flow to that region in response to a rapid loss in blood pressure in that body region. In an example, a distributed system of non-central (peripheral) blood pumps which assist in blood circulation to different body regions can be useful in combat or other environments in which there is potential rapid blood loss through injury and/or trauma. In an example, having a plurality of wearable devices at different external locations can detect rapid blood loss in a given region and adjust the corresponding blood pump to that region to reduce blood loss.

In an example, multivariate analysis of blood pressure, blood volume variation, and other biometric parameters from sensors on wearable devices at different external locations of a person's body can be analyzed in real time to detect rapid blood loss from a selected body region. In an example, rapid blood loss from a given body region can indicate hemorrhaging due to injury or trauma. In an example, blood flow to a given body region suffering from rapid blood loss due to hemorrhaging can be reduced by selective adjustment of one or more non-central (peripheral) blood pumps which control blood flow to that body region. In an example, a non-central (peripheral) blood pump can have a first operational mode in which it increases blood flow above normal flow levels and a second operational mode in which it decreases blood flow below normal flow levels. In an example, the second operational mode can be activated for a given body region when there is hemorrhaging in that body region due to injury or trauma. There must be safeguards to ensure that activation of a second operational mode does not cause undesirable blood flow reduction or tissue death, but, done properly, such as system could provide results similar to real-time application of external wound pressure to stop bleeding from wounds. This could possibly save lives.

In an example, a closed loop system for human circulatory assistance can comprise: a first wearable device (such as a ring or band) which is worn by a person on their right hand or arm which collects data on the level of a biometric parameter (such as oxygenation level) concerning their right hand or arm; a second wearable device (such as a ring or band) which is worn by a person on their left hand or arm which collects data on the level of a biometric parameter (such as oxygenation level) concerning their left hand or arm; a first implanted blood pump which selectively increases blood flow to their right hand and/or arm, wherein the operation of this first implanted blood pump is adjusted based on the level of biometric parameter (such as oxygenation level) concerning their right hand or arm; and a second implanted blood pump which selectively increases blood flow to their left hand and/or arm, wherein the operation of this second implanted blood pump is adjusted based on the level of biometric parameter (such as oxygenation level) concerning their left hand or arm.

In an example, a closed loop system for human circulatory assistance can comprise: a first wearable device (such as a band or sock) which is worn by a person on their right foot or leg which collects data on the level of a biometric parameter (such as oxygenation level) concerning their right foot or leg; a second wearable device (such as a band or sock) which is worn by a person on their left foot or leg which collects data on the level of a biometric parameter (such as oxygenation level) concerning their left foot or leg; a first implanted blood pump which selectively increases blood flow to their right foot and/or leg, wherein the operation of this first implanted blood pump is adjusted based on the level of biometric parameter (such as oxygenation level) concerning their right foot or leg; and a second implanted blood pump which selectively increases blood flow to their left foot and/or leg, wherein the operation of this second implanted blood pump is adjusted based on the level of biometric parameter (such as oxygenation level) concerning their left foot or leg.

In an example, a closed loop system for human circulatory assistance can comprise: a first wearable device (such as a ring or band) which is worn by a person on their hand or arm which collects data on the level of a biometric parameter (such as oxygenation level) concerning their hand or arm; a second wearable device (such as a band or sock) which is worn by a person on their foot or leg which collects data on the level of a biometric parameter (such as oxygenation level) concerning their foot or leg; a first implanted blood pump which selectively increases blood flow to their hand and/or arm, wherein the operation of this first implanted blood pump is adjusted based on the level of biometric parameter (such as oxygenation level) concerning their hand or arm; a second implanted blood pump which selectively increases blood flow to their foot and/or leg, wherein the operation of this second implanted blood pump is adjusted based on the level of biometric parameter (such as oxygenation level) concerning their foot or leg.

In an example, a wearable device of a system can comprise a light-emitting member (such as an LED) which is configured to direct light toward the person's body. In an example, this light can be infrared light, near-infrared light, ultraviolet light, and visible and/or white light. In an example, this light can be coherent and/or laser light. In an example, a light-energy receiver can receive this directed light after it has been reflected from, or passed through, the person's body tissue and/or fluid. In an example, data from the light-energy receiver can be analyzed to determine how the spectrum of directed light has been changed by reflection from, or passage through, the person's body tissue and/or fluid. In an example, changes in the spectrum of light energy due to interaction with a person's body tissue and/or fluid can be analyzed to measure (changes in) the chemical composition of body tissue and/or fluid.

In an example, a wearable device for collecting data on a biometric parameter can include a plurality of spectroscopic sensors. In an example, a spectroscopic sensor can further comprise one or more light-energy emitters (e.g. light sources) which direct light-energy toward a body tissue and/or fluid and one or more light-energy receivers (e.g. photoreceptors) which receive light-energy which has been transmitted through or reflected by body tissue and/or fluid. In an example, a wearable device for collecting data on a biometric parameter can include a plurality of light-energy emitters which emit light-energy toward a person's body tissue and/or fluid. In an example, a wearable device for collecting data on a biometric parameter can include a plurality of light-energy receivers which receive light-energy which has been transmitted through a person's body tissue and/or fluid or reflected by the person's body tissue and/or fluid. In an example, a wearable device for collecting data on a biometric parameter can include a plurality of light-energy emitters and a single light-energy receiver. In an example, a wearable device for collecting data on a biometric parameter can include a single light-energy emitter and a plurality of light-energy receivers.

In an example, different spectroscopic sensors in a plurality of spectroscopic sensors can differ with respect to the angles at which they direct light beams toward a person's body. In an example, a first subset of spectroscopic sensors in a plurality of spectroscopic sensors can direct light beams toward a person's body at a first angle and a second subset of spectroscopic sensors in the plurality of spectroscopic sensors can direct light beams toward the person's body at a second angle. In an example, this angle can be with respect to the proximal surface of a person's body. In an example, this angle can be with respect to the surface of a wearable device from which the light beams are emitted.

In an example, different spectroscopic sensors in a plurality of spectroscopic sensors can differ with respect to the color, frequency, and/or spectrum of light beams which they direct toward a person's body. In an example, a first subset of spectroscopic sensors in a plurality of spectroscopic sensors can direct light beams with a first color, frequency, and/or spectrum toward a person's body and a second subset of spectroscopic sensors in the plurality of spectroscopic sensors can direct light beams with a second color, frequency, and/or spectrum toward the person's body.

In an example, different spectroscopic sensors in a plurality of spectroscopic sensors can differ with respect to the power or intensity of light beams which they direct toward a person's body. In an example, a first subset of spectroscopic sensors in a plurality of spectroscopic sensors can direct light beams with a first level of power or intensity toward a person's body and a second subset of spectroscopic sensors in the plurality of spectroscopic sensors can direct light beams with a second level of power or intensity toward the person's body.

In an example, different spectroscopic sensors in a plurality of spectroscopic sensors can differ with respect to the body tissue depth from which light beams are reflected back to light-energy receivers. In an example, a first subset of spectroscopic sensors in a plurality of spectroscopic sensors can receive light beams reflected from a first tissue depth and a second subset of spectroscopic sensors in the plurality of spectroscopic sensors can receive light beams reflected from a second tissue depth.

In an example, different spectroscopic sensors in a plurality of spectroscopic sensors can differ with respect to the polarization or coherence of light beams which where they direct toward a person's body. In an example, a first subset of spectroscopic sensors in a plurality of spectroscopic sensors can direct light beams with a first orientation or degree of polarization or coherence toward a person's body and a second subset of spectroscopic sensors in the plurality of spectroscopic sensors can direct light beams with a second orientation or degree of polarization or coherence toward the person's body.

In an example, different spectroscopic sensors in a plurality of spectroscopic sensors can differ with respect to the timing and/or synchronization of light beams directed toward a person's body. In an example, light beam emission from different light-energy emitters can be sequenced and/or multiplexed. In an example, light beam emission and reception between different associated pairs of light-energy emitters and receivers can be sequenced to isolate measurement of biometric values from different tissue depths and/or locations. In an example, emission and reception of light-energy between different pairs of light-energy emitters and light-energy receivers can be multiplexed. In an example, emission of light-energy from multiple light-energy emitters can be sequenced and/or multiplexed to be received by a single light-energy receiver at different times.

In an example, different spectroscopic sensors in a plurality of spectroscopic sensors can differ with respect to the location on a person's body where they direct light beams and/or the location on a wearable device from which they emit light beams. In an example, a first subset of spectroscopic sensors in a plurality of spectroscopic sensors can direct light beams toward a first location on a person's body and a second subset of spectroscopic sensors in the plurality of spectroscopic sensors can direct light beams toward a second location on the person's body. In an example, a first subset of spectroscopic sensors in a plurality of spectroscopic sensors can direct light beams toward a first side of a portion of a person's body and a second subset of spectroscopic sensors in the plurality of spectroscopic sensors can direct light beams toward a second side of the portion of the person's body. In an example, a first subset of spectroscopic sensors in a plurality of spectroscopic sensors can direct light beams from a first location on a wearable device and a second subset of spectroscopic sensors in the plurality of spectroscopic sensors can direct light beams from a second location on a wearable device.

In an example, spectroscopic sensors in a plurality of spectroscopic sensors can be configured to be distributed around (at least half of) the circumference of a person's finger, wrist, arm, ankle, or leg. In an example, spectroscopic sensors in a plurality of spectroscopic sensors can be distributed around (at least half of) the circumference of a wearable device which encircles a portion of a person's body. In an example, spectroscopic sensors in a plurality of spectroscopic sensors can be distributed around (at least half of) the circumference of a finger ring, smart watch band, wrist band, arm band, ankle band, or smart sock.

In an example, a plurality of spectroscopic sensors can comprise a circumferential array of spectroscopic sensors around (at least half of the circumference of) a person's finger, wrist, arm, ankle, or toe. In an example, a plurality of spectroscopic sensors can comprise a ring or circle of spectroscopic sensors around (at least half of the circumference of) a person's finger, wrist, arm, ankle, or toe. In an example, a plurality of spectroscopic sensors can be incorporated into the band of a smart watch (or wrist band) or fitness band. In an example, a plurality of spectroscopic sensors can be incorporated into the side pieces of eyeglasses. In an example, a plurality of spectroscopic sensors can comprise a cylindrical matrix or grid of spectroscopic sensors.

In an example, light-energy emitters and light-energy receivers in a plurality of spectroscopic sensors can be distributed around (at least half of) the circumference of a portion of a person's body (such as a person's finger, wrist, arm, ankle, or leg) in a circumferentially-alternating manner, wherein circumferentially-alternating means repeatedly alternating between a light-energy emitter and a light-energy receiver around (at least half of) the circumference of the portion of the person's body. In an example, light-energy emitters and light-energy receivers in a plurality of spectroscopic sensors can be pair-wise associated at opposite sides of the circumference of a portion of a person's body (such as a person's finger, wrist, arm, ankle, or leg), wherein pair-wise associated at opposite sides means that each light-energy emitter is associated with a light-energy receiver which located on the (diametrically) opposite side of the body portion.

In an example, different light-energy emitters can differ with respect to the angles at which they direct light beams toward a person's body. In an example, a first subset of light-energy emitters can direct light beams toward a person's body at a first angle and a second subset of light-energy emitters can direct light beams toward the person's body at a second angle. In an example, this angle can be with respect to the proximal surface of a person's body. In an example, this angle can be with respect to the surface of a wearable device from which the light beams are emitted. In an example, a first light-energy emitter can emit light with a first light projection and/or body incidence angle and a second light-energy emitter can emit light with a second light projection and/or body incidence angle.

In an example, different light-energy emitters can differ with respect to the color, frequency, and/or spectrum of light beams which they direct toward a person's body. In an example, a first subset of light-energy emitters can direct light beams with a first color, frequency, and/or spectrum toward a person's body and a second subset of light-energy emitters can direct light beams with a second color, frequency, and/or spectrum toward the person's body. In an example, a light-energy emitter can emit light energy whose frequency and/or spectrum changes over time. In an example, a light-energy emitter can emit a sequence of light pulses at different selected frequencies.

In an example, a wearable device can comprise a first light-energy emitter and a second light-energy emitter. In an example, a first light-energy emitter can emit light with a first light frequency, color, and/or spectrum and a second light-energy emitter can emit light with a second light frequency, color, and/or spectrum. In an example, light from the first light-energy emitter can reflect primarily from a first depth, breadth, location, and/or type of body tissue and light from the second light-energy emitter can reflect primarily from a first depth, breadth, location, and/or type of body tissue. In an example, first and second light-energy emitters can emit light simultaneously. In an example, first and second light-energy emitters can emit light in a selected chronological sequence and/or timing pattern.

In an example, a first light-energy emitter can emit light-energy with a first light wavelength (or wavelength range or spectral distribution) and a second light-energy emitter can simultaneously emit light-energy with a second light wavelength (or wavelength range or spectral distribution) during the same time period. In an example, a first light-energy emitter can emit light-energy with a first light wavelength (or wavelength range or spectral distribution) and a second light-energy emitter can simultaneously emit light-energy with a second light wavelength (or wavelength range or spectral distribution) during the same time period in order to measure different physiological parameters, analytes, or conditions.

In an example, different light-energy emitters can emit light with different wavelengths or wavelength ranges based on data from one or more biometric sensors detecting different biological or physiological parameters or conditions. In an example, different emitters can emit light with different wavelengths or wavelength ranges based on data from one or more biometric sensors when a person is engaged in different types of activities. In an example, different emitters can emit light with different wavelengths or wavelength ranges based on data from one or more environmental sensors in response to different environmental parameters or conditions.

In an example, a first light-energy receiver can receive light-energy with a first light wavelength (or wavelength range or spectral distribution) and a second light-energy receiver can simultaneously receive light-energy with a second light wavelength (or wavelength range or spectral distribution) during the same time period. In an example, a first light-energy receiver can receive light-energy with a first light wavelength (or wavelength range or spectral distribution) and a second light-energy receiver can simultaneously receive light-energy with a second light wavelength (or wavelength range or spectral distribution) during the same time period in order to simultaneously measure different physiological parameters, analytes, or conditions.

In an example, a light-energy emitter can emit light-energy with a first light wavelength (or wavelength range or spectral distribution) during a first time period and can emit light-energy with a second light wavelength (or wavelength range or spectral distribution) during a second time period. In an example, a light-energy emitter can emit light-energy with a first light wavelength (or wavelength range or spectral distribution) during a first time period and can emit light-energy with a second light wavelength (or wavelength range or spectral distribution) during a second time period in order to measure different physiological parameters, analytes, or conditions. In an example, a light-energy emitter can automatically cycle through light-energy emissions with a variety of wavelengths (or wavelength ranges or spectral distributions) during different time periods in order to measure different physiological parameters, analytes, or conditions.

In an example, a light-energy emitter can emit light-energy with a first light wavelength (or wavelength range or spectral distribution) during a first time period and can emit light-energy with a second light wavelength (or wavelength range or spectral distribution) during a second time period in response to changing environmental conditions. In an example, a light-energy emitter can emit light-energy with a first light wavelength (or wavelength range or spectral distribution) during a first time period and can emit light-energy with a second light wavelength (or wavelength range or spectral distribution) during a second time period in response to changing biometric results. In an example, a light-energy emitter can emit light-energy with a first light wavelength (or wavelength range or spectral distribution) during a first time period and can emit light-energy with a second light wavelength (or wavelength range or spectral distribution) during a second time period in response to changing physiological conditions.

In an example, a light-energy receiver can receive light-energy with a first light wavelength (or wavelength range or spectral distribution) during a first time period and can receive light-energy with a second light wavelength (or wavelength range or spectral distribution) during a second time period. In an example, a light-energy receiver can receive light-energy with a first light wavelength (or wavelength range or spectral distribution) during a first time period and can receive light-energy with a second light wavelength (or wavelength range or spectral distribution) during a second time period in order to measure different physiological parameters, analytes, or conditions. In an example, a light-energy receiver can automatically cycle through light-energy emissions with a variety of wavelengths (or wavelength ranges or spectral distributions) during a different time periods in order to measure different physiological parameters, analytes, or conditions.

In an example, a light-energy receiver can receive light-energy with a first light wavelength (or wavelength range or spectral distribution) during a first time period and can receive light-energy with a second light wavelength (or wavelength range or spectral distribution) during a second time period in response to changing environmental conditions. In an example, a light-energy receiver can receive light-energy with a first light wavelength (or wavelength range or spectral distribution) during a first time period and can receive light-energy with a second light wavelength (or wavelength range or spectral distribution) during a second time period in response to changing biometric results. In an example, a light-energy receiver can receive light-energy with a first light wavelength (or wavelength range or spectral distribution) during a first time period and can receive light-energy with a second light wavelength (or wavelength range or spectral distribution) during a second time period in response to changing physiological conditions.

In an example, different light-energy emitters can differ with respect to the power or intensity of light beams which they direct toward a person's body. In an example, a first subset of light-energy emitters can direct light beams with a first level of power or intensity toward a person's body and a second subset of light-energy emitters can direct light beams with a second level of power or intensity toward the person's body.

In an example, different light-energy emitters and receivers can differ with respect to the body tissue depth from which light beams are reflected back to light-energy receivers. In an example, a first subset of light-energy receivers can receive light beams reflected from a first tissue depth and a second subset of light-energy receivers can receive light beams reflected from a second tissue depth. In an example, light from the first light-energy emitter can reflect primarily from a first depth, breadth, location, and/or type of body tissue and light from the second light-energy emitter can reflect primarily from a first depth, breadth, location, and/or type of body tissue.

In an example, different light-energy emitters can differ with respect to the polarization or coherence of light beams which where they direct toward a person's body. In an example, a first subset of light-energy emitters can direct light beams with a first orientation or degree of polarization or coherence toward a person's body and a second subset of light-energy emitters can direct light beams with a second orientation or degree of polarization or coherence toward the person's body. In an example, a wearable device can comprise a first light-energy emitter and a second light-energy emitter. In an example, the first light-energy emitter can emit light with a first light coherence, polarization, and/or phase and the second light-energy emitter can emit light with a second light coherence, polarization, and/or phase.

In an example, a wearable device can comprise an array, grid, and/or matrix of light-energy emitters which differ in one or more parameters selected from the group consisting of: location and/or distance from a light-energy receiver; distance to body surface; light beam frequency, color, and/or spectrum; light beam coherence, polarity, and/or phase; light beam power and/or intensity; light beam projection and/or body incidence angle; light beam duration; light beam size; and light beam focal distance. In an example, a system can comprise an array, grid, and/or matrix of light-energy receivers which differ in: location and/or distance from a light-energy emitter; and/or distance to body surface.

In an example, different light-energy emitters can differ with respect to the location on a person's body where they direct light beams and/or the location on a wearable device from which they emit light beams. In an example, a first subset of light-energy emitters can direct light beams toward a first location on a person's body and a second subset of light-energy emitters can direct light beams toward a second location on the person's body. In an example, a first subset of light-energy emitters can direct light beams toward a first side of a portion of a person's body and a second subset of light-energy emitters can direct light beams toward a second side of the portion of the person's body. In an example, a first subset of light-energy emitters can direct light beams from a first location on a wearable device and a second subset of light-energy emitters can direct light beams from a second location on a wearable device.

In an example, light-energy emitters and receivers can be configured to be distributed around (at least half of) the circumference of a person's finger, wrist, arm, ankle, or leg. In an example, light-energy emitters and receivers can be distributed around (at least half of) the circumference of a wearable device which encircles a portion of a person's body. In an example, light-energy emitters and receivers can be distributed around (at least half of) the circumference of a finger ring, smart watch band, wrist band, arm band, ankle band, or smart sock.

In an example, a plurality of light-energy emitters and receivers can comprise a circumferential array of light-energy emitters and receivers around (at least half of the circumference of) a person's finger, wrist, arm, ankle, or toe. In an example, a plurality of light-energy emitters and receivers can comprise a ring or circle of light-energy emitters and receivers around (at least half of the circumference of) a person's finger, wrist, arm, ankle, or toe. In an example, a plurality of light-energy emitters and receivers can be incorporated into the band of a smart watch (or wrist band) or fitness band. In an example, a plurality of light-energy emitters and receivers can be incorporated into the side pieces of eyeglasses. In an example, a plurality of light-energy emitters and receivers can comprise a cylindrical matrix or grid of spectroscopic sensors.

In an example, a wearable device can comprise a plurality of spectroscopic sensors. In an example, different spectroscopic sensors in a plurality of spectroscopic sensors can differ with respect to being at different locations around (at least half of) the circumference of a finger, wrist, arm, ankle, or toe. In an example, a plurality of spectroscopic sensors can comprise a ring of paired light-energy emitters and light-energy receivers around a finger, wrist, arm, ankle, or tow, wherein a light-energy emitter and light-energy receiver in pair are on opposite sides of the finger, wrist, arm, ankle, or tow. In an example, a plurality of spectroscopic sensors can comprise a ring of paired light-energy emitters and light-energy receivers around a finger, wrist, arm, ankle, or tow, wherein a light-energy emitter and light-energy receiver in pair are next to each other in the ring, and wherein light-energy emitters and receivers alternate around the ring. In an example, a plurality of spectroscopic sensors can comprise a plurality of light-energy emitters which are in optical communication with a single light-energy receiver. In an example, a plurality of spectroscopic sensors can comprise a single light-energy emitter which is in optical communication with a plurality of light-energy receivers.

In an example, a light-energy emitter can emit light along a first vector and a light-energy receiver can receive light along a second vector. In an example, the second vector can be substantially reversed from (e.g. 180-degree reflection) and parallel to the first vector. In an example, the second vector can be substantially perpendicular to (e.g. 90-degree angle relative to) the first vector. In an example, the second vector can be reversed from the first vector and symmetric to the first vector with respect to a virtual vector which extends outward in a perpendicular manner from the surface of a person's body. In an example, a system can include one or more light guides which direct light-energy from a first location, angle, and/or transmission vector to a second location, angle, and/or transmission vector. In an example, different light-energy emitters can emit light rays at different angles with respect to a device surface. In an example, different light-energy emitters can emit light rays at different angles with respect to a body surface. In an example, these angles can be between 60 and 120 degrees.

In an example, a spectroscopic sensor can comprise both a light-energy emitter and a light-energy receiver. In an example, a light-energy emitter and a light-energy receiver which are in optical communication with each other can comprise a spectroscopic sensor. In an example, a light-energy receiver can receive light which has been emitted by the light-energy emitter and then transmitted through or reflected from body tissue and/or fluid. In an example, a light-energy emitter and light-energy receiver can be paired such that light energy from a selected light-energy emitter is received by a selected light-energy receiver after that light energy has been transmitted through or reflected by body tissue and/or fluid.

In an example, a spectroscopic sensor can comprise a light-energy receiver alone (without a light-energy emitter) if it uses ambient light which has been reflected from or transmitted through body tissue and/or fluid. In an example, changes in the spectrum of ambient light which has been reflected from or transmitted through body tissue and/or fluid can be analyzed to measure biometric parameters with respect to the molecular composition of body tissue and/or fluid. In an example, a light-energy receiver which receives ambient light after that light has interacted with body tissue and/or fluid can be referred to as a spectroscopy sensor. In an example, an ambient light source can be solar radiation or artificial lighting in a person's environment.

In an example, a light-energy receiver can be optically isolated from light from a light-energy emitter which has not yet passed through or been reflected by body tissue and/or fluid. In an example, a light-energy receiver can be optically isolated from ambient light which has not yet passed through or been reflected by body tissue and/or fluid. In an example, a light-energy receiver can be optically isolated by means of a light blocking ring, layer, coating, cladding, or other component of the wearable device. In an example, a light-energy receiver can be optically isolated by a compressible, elastomeric, and/or inflatable ring between a light-energy receiver and the surface of a person's body. In an example, a light-energy receiver can be optically isolated by a compressible, elastomeric, and/or inflatable polygon-shaped barrier between a light-energy receiver and the surface of a person's body. In an example, a polygon-shaped barrier can have a square or hexagonal shape.

In an example, a wearable device of this system can have one or more light-energy emitters. In an example, one or more light-energy emitters can be light emitting diodes (LEDs). In an example, one or more light-energy emitters can emit coherent light. In an example, one or more light-energy emitters can be lasers. In an example, a light-energy emitter can emit infrared, near-infrared light, or ultraviolet light. In an example, a light-energy emitter can emit white light. In an example, a light-energy emitter can be selected from the group consisting of: light emitting diode (LED), coherent light source, organic light emitting diode (OLED), laser, laser diode, infrared light-energy emitter, multi-wavelength source, resonant cavity light emitting diode (RCLED), super-luminescent light emitting diode (SLED), and ultraviolet light-energy emitter. In an example, a light-energy receiver can be selected from the group consisting of: photodetector, photoresistor, avalanche photodiode (APD), charge-coupled device (CCD), complementary metal-oxide semiconductor (CMOS), infrared detector, infrared photoconductor, infrared photodiode, light dependent resistor (LDR), optoelectric sensor, photoconductor, photodiode, photomultiplier, and phototransistor.

In an example, a light-energy emitter can be a red-light laser. In an example, a light-energy emitter can be a green-light laser. In an example, a wearable device can have both a red-light laser and a green-light laser. In an example, a red light-energy emitter can be in optical communication with a first light-energy receiver (after the red light has interacted with body tissue and/or fluid) and a green light-energy emitter can be in optical communication with a second light-energy receiver (after the green light has interacted with body tissue and/or fluid). In an example, a wearable device can comprise a prism and/or filter which splits ambient light into red light and green light, wherein the red light is in optical communication with a first light-energy receiver (after the red light has interacted with body tissue and/or fluid) and the green light is in optical communication with a second light-energy receiver (after the green light has interacted with body tissue and/or fluid).

In an example, a wearable device can have a red light-energy emitter and an infrared light-energy emitter. In an example, a red light-energy emitter can be in optical communication with a first light-energy receiver (after the red light has interacted with body tissue and/or fluid) and an infrared light-energy emitter can be in optical communication with a second light-energy receiver (after the infrared light has interacted with body tissue and/or fluid).

In an example, a wearable device can have a first light-energy emitter which emits light with a wavelength of 660 nm and second light-energy emitter which emits light with a wavelength of 940 nm. In an example, a wearable device can have a first light-energy emitter which emits light with a wavelength within the range of 600 to 700 nm and second light-energy emitter which emits light with a wavelength within the range of 850 to 950 nm. In an example, the first light-energy emitter can be in optical communication with a first light-energy receiver (after its light has interacted with body tissue and/or fluid) and the second light-energy emitter can be in optical communication with a second light-energy receiver (after its light has interacted with body tissue and/or fluid). In an example, body oxygenation can be estimated based on the ratio of changes in the spectra of light beams from the two light-energy emitters due to those light beams having been transmitted through or reflected by body tissue and/or fluid.

In an example, a wearable device of this system can comprise one or more paired sets of light-energy emitters and light-energy receivers. In an example, each paired set can be configured so that light emitted from the light-energy receiver is received by the light-energy receiver after the light has been transmitted through or reflected from body tissue and/or fluid. In an example, different sets of light-energy emitters and receivers can have different locations wherein light is transmitted through or reflected by a person's body. In an example, a first pair comprising a light-energy emitter and a light-energy receiver can reflect light from a body surface at a first location and a second pair comprising a light-energy emitter and a light-energy receiver can reflect light from a body surface at a second location. In an example, different sets of light-energy emitters and receivers can have different angles at which light is transmitted through or reflected by a person's body. In an example, a first pair comprising a light-energy emitter and a light-energy receiver can reflect light from a body surface at a first angle and a second pair comprising a light-energy emitter and a light-energy receiver can reflect light from a body surface at a second angle.

In an example, pairs of light-energy emitters and light-energy receivers can be distributed around the circumference of a wearable device (such as a finger ring, watch band, wrist band, arm band, or ankle band) such that at least one pair is in close contact with the surface of a person's body regardless of rotation and/or shifting of the wearable device. In an example, pairs of light-energy emitters and light-energy receivers can be distributed around the circumference of a wearable device (such as a finger ring, watch band, wrist band, arm band, or ankle band) such that the light beam from at least one light-energy emitter is substantially perpendicular to the proximal surface of a person's body regardless of rotation and/or shifting of the wearable device.

In an example, a wearable device of this system can include one or more light-blocking layers, coatings, or claddings. In an example, a wearable device can include one or more light-reflecting layers, coatings, or claddings. In an example, a wearable device can include one or more mirrors. In an example, a light-blocking and/or light-reflecting layer, coating, and/or cladding can be opaque. In an example, a light-blocking and/or light-reflecting layer, coating, and/or cladding can comprise a black or sliver coating. In an example, a light-blocking and/or light-reflecting layer, coating, and/or cladding can be Mylar. In an example, a light-blocking and/or light-reflecting layer, coating, and/or cladding can prevent the direct transmission of light from a light-energy emitter to a light-energy receiver apart from transmission through or reflection from body tissue and/or fluid. In an example, a light-blocking and/or light-reflecting layer, coating, and/or cladding can optically isolate a light-energy receiver from ambient light. In an example, a light-blocking and/or light-reflecting layer, coating, and/or cladding can reduce or prevent the direct transmission of ambient light to a light-energy receiver apart from transmission through or reflection from body tissue and/or fluid.

In an example, a wearable device of this system can include a light barrier between a light-energy emitter and a light-energy receiver which reduces or eliminates the direct transmission of light energy from the emitter to the receiver. In an example, a light barrier can be located between a light-energy receiver and a person's skin. In an example, a light barrier can be opaque. In an example, a light barrier can be compressible, flexible, and/or elastic. In an example, a light barrier can comprise compressible foam. In an example, a light barrier can be an inflatable member (such as a balloon) which is filled with a gas or liquid. In an example, a light barrier can have a linear shape. In an example, a light barrier can have a circular, elliptical, sinusoidal, or other arcuate shape. In an example, a light barrier can surround a light-energy receiver. In an example, a light barrier can surround a light-energy emitter.

In an example, a wearable device of this system can include one or more light filters. In an example, a light filter can partially absorb and/or block light transmission between a light-energy emitter and body tissue. In an example, a light filter can partially absorb and/or block light transmission between ambient light and body tissue. In an example, a light filter can partially absorb and/or block light transmission between body tissue and a light-energy receiver. In an example, a wearable device can comprise two or more light filters which are alternately moved into the path of light beams from a light-energy emitter. In an example, one or more light filters can partially absorb and/or block one or more selected light wavelengths, wavelength ranges, frequencies, and/or frequency ranges. In an example, a light filter may absorb and/or block infrared or ultraviolet light. In an example, a light filter can selectively allow transmission of only infrared light or only ultraviolet light. In an example, a light filter can be made from one or more materials selected from the group consisting of: acrylic, crystal, glass, high-durometer plastic, low-durometer plastic, optical-pass material, polycarbonate, polyethylene, polymer, polyurethane, resin, sapphire, and transparent polymer. In an example, a light filter can be made by adding a light-absorbing dye to acrylic, crystal, glass, plastic, polycarbonate, polyethylene, polymer, polyurethane, resin, and/or a transparent polymer.

In an example, a wearable device of this system can include one or more lenses. In an example, a wearable device can include a lens which selectively refracts and/or focuses light. In an example, a lens can selectively refract and/or focus light transmission between a light-energy emitter and body tissue. In an example, a lens can selectively refract and/or focus light transmission between ambient light and body tissue. In an example, a lens can selectively refract and/or focus light transmission between body tissue and a light-energy receiver. In an example, a lens can be selected from the group consisting of: biconcave, biconvex, collimating, columnar, concave, converging, convex, diverging, fluid lens, Fresnel, multiple lenses, negative meniscus, planoconcave, planoconvex, polarizing, positive meniscus, prismatic, and variable-focal lens. In an example, a lens can be made from one or more materials selected from the group consisting of: acrylic, crystal, glass, high-durometer plastic, low-durometer plastic, optical-pass material, polycarbonate, polyethylene, polymer, polyurethane, resin, sapphire, and transparent polymer.

In an example, a wearable device of this system can include a light guide. In an example, a light guide can be flexible. In an example, a light guide can be generally cylindrical and/or columnar. In an example, a light guide can have a refractive index of at least 3.141. In an example, a light guide can be made from one or more materials selected from the group consisting of: acrylic, crystal, elastomeric light-transmissive material, glass, high-durometer plastic, low-durometer plastic, optical-pass material, polycarbonate, polyethylene, polymer, polyurethane, resin, sapphire, and transparent polymer.

In an example, a plurality of light-energy emitters can co-linear. In an example, a plurality of light-energy emitters and a light-energy receiver can be co-linear. In an example, a plurality of light-energy emitters can be configured in a polygonal array in proximity to a light-energy receiver. In an example, a plurality of light-energy emitters can be configured in a polygonal array which includes a light-energy receiver. In an example, a plurality of light-energy emitters can be configured in a polygonal array around a light-energy receiver. In an example, a plurality of light-energy emitters can be configured in a circular array in proximity to a light-energy receiver. In an example, a plurality of light-energy emitters can be configured in a circular array around a light-energy receiver. In an example, a plurality of light-energy emitters can emit light in a circular sequence around a central light-energy receiver.

In an example, an array of light-energy emitters can have a square or rectangular shape. In an example, an array of light-energy emitters can have a hexagonal shape. In an example, an array of light-energy emitters can have a circular shape. In an example, an array of light-energy emitters can have a sunburst (e.g. radial spoke) shape. In an example, an array of light-energy emitters can have a cylindrical and/or ring shape. In an example, an array of light-energy emitters and receivers can have a square or rectangular shape. In an example, an array of light-energy emitters and receivers can have a hexagonal shape. In an example, an array of light-energy emitters and receivers can have a circular shape. In an example, an array of light-energy emitters and receivers can have a sunburst (e.g. radial spoke) shape. In an example, an array of light-energy emitters and receivers can have a cylindrical and/or ring shape.

In an example, the depths, breadths, locations, and/or types of body tissue or fluid from which light beams from a plurality of light-energy emitters are reflected can be determined by a selected geometric configuration of the plurality of light-energy emitters and a light-energy receiver. In an example, a selected geometric configuration of a plurality of light-energy emitters and a light-energy receiver can be designed to most accurately measure an analyte level in the body. In an example, the geometric configuration of a plurality of light-energy emitters and a light-energy receiver can be adjusted automatically (in an iterative manner) by a system in order to more accurately measure an analyte level in the body for a specific person, for a specific type of activity, or for a specific configuration of the system relative to the person's body surface.

In an example, the geometric configuration of a plurality of light-energy emitters and a light-energy receiver can be adjusted automatically to maintain accurate measurement of an analyte level in the body even if the system shifts and/or moves relative to the person's body surface. In an example, a system can automatically vary the geometric configuration of a plurality of light-energy emitters and a light-energy receiver in order to scan through a range of tissue depths, locations, and/or types in order to measure an analyte level in the body more accurately. In an example, a plurality of light-energy emitters can emit light simultaneously. In an example, a plurality of light-energy emitters can emit light in a selected chronological sequence and/or timing pattern.

In an example, a wearable device can include a linear array, grid, and/or matrix of light-energy emitters. In an example, a wearable device can include a rectangular array, grid, and/or matrix of light-energy emitters. In an example, a wearable device can include a circular or elliptical array, grid, and/or matrix of light-energy emitters. In an example, a wearable device can include a checkerboard array, grid, and/or matrix of light-energy emitters. In an example, a wearable device can include a three-dimensional stacked array, grid, and/or matrix of light-energy emitters. In an example, a wearable device can include a sunburst and/or radial-spoke array, grid, and/or matrix of light-energy emitters. In an example, a wearable device can include a sinusoidal array, grid, and/or matrix of light-energy emitters.

In an example, a wearable device can include a linear array, grid, and/or matrix of light-energy receivers. In an example, a wearable device can include a rectangular array, grid, and/or matrix of light-energy receivers. In an example, a wearable device can include a circular or elliptical array, grid, and/or matrix of light-energy receivers. In an example, a wearable device can include a checkerboard array, grid, and/or matrix of light-energy receivers. In an example, a wearable device can include a three-dimensional stacked array, grid, and/or matrix of light-energy receivers. In an example, a wearable device can include a sunburst and/or radial-spoke array, grid, and/or matrix of light-energy receivers. In an example, a wearable device can include a sinusoidal array, grid, and/or matrix of light-energy receivers.

In an example, a wearable device can include a linear array, grid, and/or matrix of (alternating) light-energy emitters and receivers. In an example, a wearable device can include a rectangular array, grid, and/or matrix of (alternating) light-energy emitters and receivers. In an example, a wearable device can include a circular or elliptical array, grid, and/or matrix of (alternating) light-energy emitters and receivers. In an example, a wearable device can include a checkerboard array, grid, and/or matrix of (alternating) light-energy emitters and receivers. In an example, a wearable device can include a three-dimensional stacked array, grid, and/or matrix of (alternating) light-energy emitters and receivers. In an example, a wearable device can include a sunburst and/or radial-spoke array, grid, and/or matrix of (alternating) light-energy emitters and receivers. In an example, a wearable device can include a sinusoidal array, grid, and/or matrix of (alternating) light-energy emitters and receivers.

In an example, a wearable device for collecting data on a biometric parameter concerning a person's body can include a first light-energy emitter and a second light-energy emitter. In an example, the first light-energy emitter can have a first location relative to the person's body and the second light-energy emitter can have a second location relative to the person's body. In an example, the first light-energy emitter can emit light at a first angle with respect to the surface of a person's body and the second light-energy emitter can emit light at a second angle with respect to the surface of a person's body. In an example, the first light-energy emitter can emit light with a first wavelength (or spectral distribution) and the second light-energy emitter can emit light with a second wavelength (or spectral distribution).

In an example, a system can have two (or more) light-energy emitters. In an example, a first light-energy emitter can be separated from a second light-energy emitter by a selected distance. In an example, this selected distance can be expressed in inches and be within the range of 1/16″ to 2″. In an example, this selected distance can be expressed in metric units and be within the range of 2 mm to 5 cm. In an example, if this distance is along a circumferential axis, this selected distance can be expressed in (compass or polar coordinate) degrees and be within the range of 2 degrees to 60 degrees.

In an example, a light-energy emitter can be part of an arcuate band. In an example, a light-energy emitter can be part of a housing which is held on a person's body by an arcuate band. In an example, a system can comprise an array, grid, and/or matrix of two or more light-energy emitters with a proximal-to-distal orientation. In an example, a system can comprise an array, grid, and/or matrix of two or more light-energy emitters along a proximal-to-distal axis. In an example, a system can comprise an array, grid, and/or matrix of two or more light-energy emitters with a circumferential orientation. In an example, a system can comprise an array, grid, and/or matrix of two or more light-energy emitters along a circumferential axis.

In an example, a system can have two (or more) light-energy receivers. In an example, a first light-energy receiver can be separated from a second light-energy receiver by a selected distance. In an example, this selected distance can be expressed in inches and be within the range of 1/16″ to 2″. In an example, this selected distance can be expressed in metric units and be within the range of 2 mm to 5 cm. In an example, if this distance is along a circumferential axis, this selected distance can be expressed in (compass or polar coordinate) degrees and be within the range of 2 degrees to 60 degrees.

In an example, a light-energy receiver can be part of an arcuate band. In an example, a light-energy receiver can be part of a housing which is held on a person's body by an arcuate band. In an example, a system can comprise an array, grid, and/or matrix of two or more light-energy receivers with a proximal-to-distal orientation. In an example, a system can comprise an array, grid, and/or matrix of two or more light-energy receivers along a proximal-to-distal axis. In an example, a system can comprise an array, grid, and/or matrix of two or more light-energy receivers with a circumferential orientation. In an example, a system can comprise an array, grid, and/or matrix of two or more light-energy receivers along a circumferential axis.

In an example, a light-energy emitter can emit light from the inward side of a wearable device toward the surface of a person's body (e.g. finger, wrist, arm, ear, or leg). In an example, a light-energy receiver can receive light into the inward side of a wearable device which has been transmitted through or reflected by body tissue and/or fluid. In an example, there can be a flexible and/or compressible light barrier between a light-energy emitter and a light-energy receiver. In an example, a light-energy emitter and a light-energy receiver can be on the same circumferential line (e.g. circle) of a wearable device, but at different radial locations around this circumference. In an example, a light-energy emitter and a light-energy receiver can be on the same radial location around a wearable device, but on different circumferential lines (e.g. circles).

In an example, an array of emitters and/or receivers can have a circumferential axis and a proximal-to-distal axis. In an example, this array can have at least three emitters and/or receivers along a circumferential axis and at least two emitters and/or receivers along a proximal-to-distal axis. In an example, an array can be formed from a plurality of sets of emitters and receivers, wherein each set forms the vertexes of a square or rectangle. In an example, an array can be formed from a plurality of sets of emitters and receivers, wherein each set forms the vertexes of a hexagon. In an example, an array can be formed from a plurality of sets of emitters and receivers, wherein each set forms a circle.

In an example, an array, grid, and/or matrix of two or more light-energy emitters can span up to 10% of the cross-sectional circumference of a part of a person's body such as a finger, wrist, arm, ankle, or leg. In an example, an array, grid, and/or matrix of two or more light-energy emitters can span between 10% and 25% of the cross-sectional circumference of a part of a person's body such as a finger, wrist, arm, ankle, or leg. In an example, an array, grid, and/or matrix of two or more light-energy emitters can span between 25% and 50% of the cross-sectional circumference of a part of a person's body such as a finger, wrist, arm, ankle, or leg. In an example, an array, grid, and/or matrix of two or more light-energy emitters can span between 50% and 100% of the cross-sectional circumference of a part of a person's body such as a finger, wrist, arm, ankle, or leg.

In an example, an array, grid, and/or matrix of two or more light-energy receivers can span up to 10% of the cross-sectional circumference of a part of a person's body such as a finger, wrist, arm, ankle, or leg. In an example, an array, grid, and/or matrix of two or more light-energy receivers can span between 10% and 25% of the cross-sectional circumference of a part of a person's body such as a finger, wrist, arm, ankle, or leg. In an example, an array, grid, and/or matrix of two or more light-energy receivers can span between 25% and 50% of the cross-sectional circumference of a part of a person's body such as a finger, wrist, arm, ankle, or leg. In an example, an array, grid, and/or matrix of two or more light-energy receivers can span between 50% and 100% of the cross-sectional circumference of a part of a person's body such as a finger, wrist, arm, ankle, or leg.

In an example, an array, grid, and/or matrix of (alternating) light-energy emitters and receivers can span up to 10% of the circumference of a part of a person's body such as a finger, wrist, arm, ankle, or leg. In an example, an array, grid, and/or matrix of (alternating) light-energy emitters and receivers can span between 10% and 25% of the circumference of a part of a person's body such as a finger, wrist, arm, ankle, or leg. In an example, an array, grid, and/or matrix of (alternating) light-energy emitters and receivers can span between 25% and 50% of the circumference of a part of a person's body such as a finger, wrist, arm, ankle, or leg. In an example, an array, grid, and/or matrix of (alternating) light-energy emitters and receivers can span between 50% and 100% of the circumference of a part of a person's body such as a finger, wrist, arm, ankle, or leg.

In an example, compass coordinates can be defined for the circumference of a wearable device with the 0-degree point being the most ventral point when the wearable device is worn, the 90-degree point being one-quarter of the way around the circumference in a clockwise direction from the 0-degree point, the 180-degree point being opposite the 0-degree point, and the 270-degree point being one-quarter of the way around the circumference in a clockwise direction from the 180-degree point. In an example, a light-energy emitter can be separated from a light-energy receiver by between 1 and 15 degrees. In an example, a light-energy emitter can be separated from a light-energy receiver by between 10 and 45 degrees. In an example, a light-energy emitter can be separated from a light-energy receiver by more than 44 degrees. In an example, a light-energy emitter can be separated from a light-energy receiver by 45, 60, 90, or 180 degrees. In an example, a plurality of light-energy receivers can be distributed around (at least half of) the circumference of a wearable device, being pair-wise separated from each other by between 10 and 45 degrees. In an example, a plurality of light-energy receivers can be distributed around (at least half of) the circumference of a wearable device, being pair-wise separated from each other by 45, 60, 90, or 180 degrees.

In an example, a system can have a circumferential array, matrix, or grid of four or more emitters, each of which is separated from the nearest other emitter by a distance within the range of 1/16″ to 2″. In an example, a system can have a circumferential array, matrix, or grid of four or more emitters, each of which is separated from the nearest other emitter by a distance within the range of 2 mm to 5 cm. In an example, a system can have a circumferential array, matrix, or grid of four or more emitters, each of which is separated from the nearest other emitter by a distance within the range of 2 degrees to 60 degrees. In an example, a system can have a circumferential array of emitters which spans between 25% and 100% of the cross-sectional perimeter circumference of a part of the body (e.g. finger, wrist, arm, ankle, or leg) to which the system is attached. In an example, this circumferential array of emitters can be even spaced or distributed, with the same pair-wise distance or number of degrees between adjacent emitters.

In an example, a system can have a circumferential array, matrix, or grid of four or more receivers, each of which is separated from the nearest other receiver by a distance within the range of 1/16″ to 2″. In an example, a system can have a circumferential array, matrix, or grid of four or more receivers, each of which is separated from the nearest other receiver by a distance within the range of 2 mm to 5 cm. In an example, a system can have a circumferential array, matrix, or grid of four or more receivers, each of which is separated from the nearest other receiver by a distance within the range of 2 degrees to 60 degrees. In an example, a system can have a circumferential array of receivers which spans between 25% and 100% of the cross-sectional perimeter circumference of a part of the body (e.g. finger, wrist, arm, ankle, or leg) to which the system is attached. In an example, this circumferential array of receivers can be even spaced or distributed, with the same pair-wise distance or number of degrees between adjacent receivers.

In an example, different light-energy emitters and receivers can differ with respect to the timing and/or synchronization of light beams directed toward a person's body. In an example, light beam emission from different light-energy emitters can be sequenced and/or multiplexed. In an example, light beam emission and reception between different associated pairs of light-energy emitters and receivers can be sequenced to isolate measurement of biometric values from different tissue depths and/or locations. In an example, emission and reception of light-energy between different pairs of light-energy emitters and light-energy receivers can be multiplexed. In an example, emission of light-energy from multiple light-energy emitters can be sequenced and/or multiplexed to be received by a single light-energy receiver at different times.

In an example, a first light-energy emitter can emit light during a first time period and a second light-energy emitter can emit light during a second time period. In an example, a first light-energy receiver can receive light during a first time period and the second light-energy receiver can receive light during a second time period. In an example, the first light-energy emitter can emit light during a first environmental condition and the second light-energy emitter can emit light during a second environmental condition. In an example, the first light-energy emitter can emit light when the person is engaged in a first type of physical activity and the second light-energy emitter can emit light when the person is engaged in a second type of physical activity.

In an example, the angle of a beam of light emitted from a light-energy emitter can be changed over time to create a chronological sequence of beams of light with different projection and/or body incidence angles. In an example, the power or intensity of a beam of light emitted from a light-energy emitter can be changed over time to create a chronological sequence of beams of light with different power or intensity levels. Such sequences can help to more accurately measure an analyte level in the body.

In an example, the depth, breadth, location, and/or type of body tissue or fluid from which light from a light-energy emitter is reflected can be changed by adjusting the coherence, polarization, and/or phase of light emitted from the light-energy emitter. In an example, the coherence, polarization, and/or phase of light emitted from the light-energy emitter can be adjusted in order to more accurately measure an analyte level in the body. In an example, the coherence, polarization, and/or phase of light emitted from the light-energy emitter can be adjusted automatically (in an iterative manner) by a system in order to more accurately measure an analyte level in the body for a specific person, for a specific type of activity, or for a specific configuration of the system relative to the person's body surface.

In an example, the coherence, polarization, and/or phase of light emitted from the light-energy emitter can be adjusted automatically to maintain accurate measurement of an analyte level in the body even if the system shifts and/or moves relative to the person's body surface. In an example, a system can automatically vary the coherence, polarization, and/or phase of light from a light-energy emitter to scan through a range of tissue depths, locations, and/or types in order to obtain more accurate measurement of an analyte level in the body. In an example, a system can further comprise one or more optical filters or lenses which change the coherence, polarization, and/or phase of light emitted by a light-energy emitter.

In an example, the depth, breadth, location, and/or type of body tissue or fluid from which light from a light-energy emitter is reflected can be changed by adjusting the frequency, color, and/or spectrum of light emitted from the light-energy emitter. In an example, the frequency, color, and/or spectrum of light emitted from the light-energy emitter can be adjusted in order to more accurately measure an analyte level in the body. In an example, the frequency, color, and/or spectrum of light emitted from the light-energy emitter can be adjusted automatically (in an iterative manner) by a system in order to more accurately measure an analyte level in the body for a specific person, for a specific type of activity, or for a specific configuration of the system relative to the person's body surface.

In an example, the frequency, color, and/or spectrum of light emitted from the light-energy emitter can be adjusted automatically to maintain accurate measurement of an analyte level in the body even if the system shifts and/or moves relative to the person's body surface. In an example, a system can automatically vary the frequency, color, and/or spectrum of light from a light-energy emitter to scan through a range of tissue depths, locations, and/or types in order to obtain more accurate measurement of an analyte level in the body. In an example, a system can further comprise one or more optical filters or lenses which change the frequency, color, and/or spectrum of light emitted by a light-energy emitter. In an example, the frequency, color, and/or spectrum of a beam of light emitted from a light-energy emitter can be changed over time to create a chronological sequence of beams of light with different frequencies, colors, and/or spectrums.

In an example, the frequency, color, and/or spectrum of a beam of light emitted from a light-energy emitter can be changed in response to specific environmental conditions (e.g. temperature or humidity) and/or specific activities in which the person wearing a system is engaged (e.g. high level of movement, eating, sleeping, etc.) in order to more accurately measure an analyte level in the body. In an example, the projection angle of a beam of light emitted from a light-energy emitter can be changed in response to specific environmental conditions (e.g. temperature or humidity) and/or specific activities in which the person wearing a system is engaged (e.g. high level of movement, eating, sleeping, etc.) in order to more accurately measure an analyte level in the body. In an example, the power and/or intensity of a beam of light emitted from a light-energy emitter can be changed in response to specific environmental conditions (e.g. temperature or humidity) and/or specific activities in which the person wearing a system is engaged (e.g. high level of movement, eating, sleeping, etc.) in order to more accurately measure an analyte level in the body.

In an example, the geometric configuration of a light-energy emitter and a plurality of light-energy receivers can be adjusted automatically (in an iterative manner) by a system in order to more accurately measure an analyte level in the body for a specific person, for a specific type of activity, or for a specific configuration of the system relative to the person's body surface. In an example, the geometric configuration of a light-energy emitter and a plurality of light-energy receivers can be adjusted automatically to maintain accurate measurement of an analyte level in the body even if the system shifts and/or moves relative to the person's body surface. In an example, a system can automatically vary the geometric configuration of a light-energy emitter and a plurality of light-energy receivers in order to scan through a range of tissue depths, locations, and/or types in order to measure an analyte level in the body more accurately.

In an example, a light-energy emitter (or light-energy receiver) can be automatically moved relative to a wearable housing which holds it. In an example, a light-energy emitter (or light-energy receiver) can be automatically tilted, rotated, raised, or lowered by an actuator. In an example, a light-energy emitter (or light-energy receiver) can be automatically tilted, rotated, raised, or lowered if the wearable housing which holds it moves relative to the body surface on which it is worn. In an example, a light-energy emitter (or light-energy receiver) can be automatically tilted, rotated, raised, or lowered in order to maintain a selected distance (or distance range) from the surface of a person's body. In an example, a light-energy emitter (or light-energy receiver) can be automatically tilted, rotated, raised, or lowered in order to maintain a selected angle (or angle range) with respect to the surface of a person's body.

In an example, a wearable device can further comprise a rotating member which holds a light-energy emitter, a light-energy receiver, or both. In an example, rotation of this member can be done manually. In an example, this rotation can be done automatically by one or more actuators. In an example, the distance between a light-energy emitter and a light-energy receiver can be adjusted by rotating the rotating member. In an example, the location of a light-energy emitter and/or a light-energy receiver relative to a person's body can be adjusted by rotating the rotating member. In an example, movement of a light-energy emitter, a light-energy receiver, or both by a rotating member can enable more accurate measurement of an analyte level in the body. In an example, such movement of a light-energy emitter, a light-energy receiver, or both can enable customization of a system to the anatomy of a specific person for more accurate measurement of that person's analyte level.

In an example, the depth, breadth, location, and/or type of body tissue or fluid from which light from a light-energy emitter is reflected can be changed by adjusting the angle of light emitted from the light-energy emitter. In an example, the angle of light emitted from the light-energy emitter can be adjusted in order to more accurately measure an analyte level in the body. In an example, the angle of light emitted from the light-energy emitter can be adjusted automatically (in an iterative manner) by a system in order to more accurately measure an analyte level in the body for a specific person, for a specific type of activity, or for a specific configuration of the system relative to the person's body surface. In an example, the angle of light emitted from the light-energy emitter can be adjusted automatically to maintain accurate measurement of an analyte level in the body even if the system shifts and/or moves relative to the person's body surface. In an example, a system can automatically vary the angle of light from a light-energy emitter to scan through a range of tissue depths, locations, and/or types in order to obtain more accurate measurement of an analyte level in the body. In an example, a system can further comprise one or more optical filters or lenses which change the projection and/or body incidence angle of a light beam emitted by a light-energy emitter.

In an example, the depth, breadth, location, and/or type of body tissue or fluid from which light from a light-energy emitter is reflected can be changed by adjusting the power and/or intensity of light emitted from the light-energy emitter. In an example, the power and/or intensity of light emitted from the light-energy emitter can be adjusted in order to more accurately measure an analyte level in the body. In an example, the power and/or intensity of light emitted from the light-energy emitter can be adjusted automatically (in an iterative manner) by a system in order to more accurately measure an analyte level in the body for a specific person, for a specific type of activity, or for a specific configuration of the system relative to the person's body surface. In an example, the power and/or intensity of light emitted from the light-energy emitter can be adjusted automatically to maintain accurate measurement of an analyte level in the body even if the system shifts and/or moves relative to the person's body surface. In an example, a system can automatically vary the power and/or intensity of light from a light-energy emitter to scan through a range of tissue depths, locations, and/or types in order to obtain more accurate measurement of an analyte level in the body.

In an example, the depth, breadth, location, and/or type of body tissue or fluid from which light from a light-energy emitter is reflected and received by a light-energy receiver can be changed by adjusting the distance between a light-energy emitter and a light-energy receiver. In an example, the distance between a light-energy emitter and a light-energy receiver can be adjusted in order to more accurately measure an analyte level in the body. In an example, the distance between a light-energy emitter and a light-energy receiver can be adjusted automatically (in an iterative manner) by a system in order to more accurately measure an analyte level in the body for a specific person, for a specific type of activity, or for a specific configuration of the system relative to the person's body surface. In an example, the distance between a light-energy emitter and a light-energy receiver can be adjusted automatically to maintain accurate measurement of an analyte level in the body even if the system shifts and/or moves relative to the person's body surface. In an example, a system can automatically vary the distance between a light-energy emitter and a light-energy receiver to scan through a range of tissue depths, locations, and/or types in order to obtain more accurate measurement of an analyte level in the body.

In an example, a wearable device can further comprise a track, channel, or slot along which a light-energy emitter, a light-energy receiver, or both can be moved. In an example, this movement can be done automatically by one or more actuators. In an example, this track, channel, or slot can have a circumferential orientation. In an example, this track, channel, or slot can have a proximal-to-distal orientation. In an example, the distance between a light-energy emitter and a light-energy receiver can be adjusted by moving the emitter, the receiver, or both along such a track, channel, or slot. In an example, the location of a light-energy emitter and/or a light-energy receiver relative to a person's body can be adjusted by moving the emitter, the receiver, or both along such a track, channel, or slot. In an example, movement of a light-energy emitter, a light-energy receiver, or both along a track, channel, or slot can enable more accurate measurement of an analyte level in the body. In an example, movement of a light-energy emitter, a light-energy receiver, or both along a track, channel, or slot can enable customization of a wearable device to the anatomy of a specific person for more accurate measurement of that person's analyte level.

In an example, a beam of light can: be emitted by the light-energy emitter along a first vector; hit body tissue; reflect back from the body tissue; pass through a lens or light guide; and enter the light-energy receiver along a second vector which is reversed from and parallel to the first vector. In an example, a beam of light can: be emitted by the light-energy emitter along a first vector; hit body tissue; reflect back from the body tissue; pass through a rotating and/or tilting lens or light guide; and enter the light-energy receiver along a second vector which is reversed from and parallel to the first vector. In an example, a beam of light can: be emitted by the light-energy emitter along a first vector; hit body tissue; reflect back from the body tissue; pass through a lens or light guide which is rotated and/or tilted by an actuator; and enter the light-energy receiver along a second vector which is reversed from and parallel to the first vector.

In an example, the beam of light emitted by a light-energy emitter can be automatically moved by using an actuator to automatically move a lens (or light guide) through which this beam is transmitted. In an example, the beam of light emitted by a light-energy emitter can be automatically moved by using an actuator to automatically rotate, tilt, raise, or lower a lens (or light guide) through which this beam is transmitted. In an example, the beam of light emitted by a light-energy emitter can be automatically moved by using an actuator to automatically change the focal distance of a lens (or light guide) through which this beam is transmitted. In an example, the beam of light emitted by a light-energy emitter can be automatically moved by using an actuator to automatically move a light guide through which this beam is transmitted. In an example, the beam of light emitted by a light-energy emitter can be automatically moved by using an actuator to automatically rotate, tilt, raise, or lower a light guide through which this beam is transmitted. In an example, the beam of light emitted by a light-energy emitter can be automatically moved by using an actuator to automatically move a light reflector (such as a mirror) from which this beam is reflected. In an example, the beam of light emitted by a light-energy emitter can be automatically moved by using an actuator to automatically rotate, tilt, raise, or lower a light reflector (such as a mirror) from which this beam is reflected.

In an example, a blood pump can be incorporated into an artificial blood flow lumen and/or vessel. In an example, an artificial blood flow lumen and/or vessel can be implanted into fluid communication with a natural blood vessel by one or more connecting members or connection methods selected from the group consisting of: endovascular and/or transluminal insertion and expansion, surgical anastomosis, surgical sutures, purse string suture, drawstring, pull tie, friction fit, surgical staples, tissue adhesive, gel, fluid seal, chemical bonding, cauterization, blood vessel connector and/or joint, vessel branch, twist connector, helical threads or screw connector, connection port, interlocking joints, tongue and groove connection, flanged connector, beveled ridge, magnetic connection, plug connector, circumferential ring, inflatable ring, and snap connector.

In an example, an implanted blood flow lumen can be selected from the group consisting of: artificial vessel segment, bioengineered vessel segment, transplanted vessel segment, artificial vessel joint, vessel branch, stent or other expandable mesh or framework, artificial lumen, manufactured catheter, manufactured tube, valve, vessel valve segment, multi-channel lumen, blood pump housing, and elastic blood chamber. In an example, an implanted blood flow lumen can have a longitudinal axis which is relatively straight. In an example, an implanted blood flow lumen can have a longitudinal axis which is arcuate. In an example, an implanted blood flow lumen can have a longitudinal axis which follows the shape of longitudinal axis of the natural blood vessel with which the implanted blood flow lumen is in fluid communication.

In another example, an implanted blood flow lumen containing a blood pump can spliced into a natural blood vessel (from an upstream location to a downstream location) so as to entirely replace a longitudinal segment of the natural blood vessel. An advantage of this splicing approach is that blood flow need not be bifurcated; this can reduce potential thrombogenesis from flow junctures. Even when blood flows are divided among multiple intra-luminal channels within an implanted blood flow lumen, there is greater design flexibility in an entirely-manufactured blood flow lumen. This design flexibility can be used to create hemodynamic flow patterns which minimize thrombogenesis despite the splitting of blood flows. A potential disadvantage of this splicing approach is that it involves the removal of a longitudinal segment of the natural blood vessel.

In another example, an implanted blood flow lumen can be configured to be implanted at least partially outside the walls of the natural blood vessel with which the implanted blood flow lumen is in fluid communication. In an example, an implanted blood flow lumen can bifurcate (and then reconverge) blood flow from an upstream location to a downstream location. In an example, an implanted blood flow lumen can divide pre-implantation blood flow through a natural blood vessel from an upstream location to a downstream location into a first blood flow and a second blood flow. In an example, these two blood flows can flow in parallel (in terms of flow dynamics even if not parallel in terms of geometry) for a while. In an example, these first and second flows can diverge at an upstream location and then reconverge at a downstream location.

In an example, this system can further comprise one or more components selected from the group consisting of: accelerometer, augmented reality eyewear, chemiresistor, electromagnetic energy sensor, human-to-computer interface, photodetector, acoustic energy sensor, breathing rate sensor, digital camera, galvanic skin response (GSR) sensor, microphone, solar panel, VR eyewear, brain oxygenation sensor, deely bobbers, eye muscle (EOG) sensor, microchip, skin conductance sensor, vibrating component, and drug pump.

In an example data from wearable sensors can be analyzed using an analytical method selected from the group consisting of: Analysis of Variance (ANOVA), Artificial Neural Network (ANN), Auto-Regressive (AR) Modeling, Bayesian Analysis, Bonferroni Analysis (BA), Centroid Analysis, Chi-Squared Analysis, Cluster Analysis, Correlation, Covariance, Data Normalization (DN), Decision Tree Analysis (DTA), Discrete Fourier transform (DFT), Discriminant Analysis (DA), Edgar AI Analysis, Carlavian Curve Analysis (CCA), and Empirical Mode Decomposition (EMD).

In an example, this system can further comprise one or more components selected from the group consisting of: barometric pressure sensor, chewing sensor, electromyographic (EMG) sensor, hydration sensor, photoplethysmography (PPG) sensor, stretch sensor, allergen sensor, capacitive sensor, electrical resistance sensor, gyroscope, multi-axial accelerometer, drug reservoir, battery, cholesterol sensor, electronic tablet, hygrometry sensor, piezoelectric sensor, swallow sensor, bend sensor, chromatographic sensor, and electronically-functional eyewear.

In an example data from wearable sensors can be analyzed using an analytical method selected from the group consisting of: Factor Analysis (FA), Fast Fourier Transform (FFT), Feature Vector Analysis (FVA), Fisher Linear Discriminant, Fourier Transformation (FT) Method, Fuzzy Logic (FL) Modeling, Gaussian Model (GM), Generalized Auto-Regressive Conditional Heteroscedasticity (GARCH) Modeling, Hidden Markov Model (HMM), Independent Components Analysis (ICA), Inter-Band Power Ratio, Inter-Channel Power Ratio, Inter-Montage Power Mean, Inter-Montage Ratio, Kalman Filter (KF), Kernel Estimation, Laplacian Filter, and Laplacian Montage Analysis.

In an example, this system can further comprise one or more components selected from the group consisting of: impedance sensor, piezoresistive sensor, sweat sensor, biochemical sensor, compass, electrophoresis sensor, inclinometer, pneumography sensor, temperature sensor, ambient temperature sensor, cardiotachometer, electromagnetic actuator, home automation control system, oximeter, blood glucose sensor, computer-to-human interface, energy transducer to generate energy from ambient electromagnetic energy, and inertial sensor.

In an example data from wearable sensors can be analyzed using an analytical method selected from the group consisting of: Least Squares Estimation, Linear Regression, Linear Transform, Logit Model, Machine Learning (ML), Markov Model, Maximum Entropy Modeling, Maximum Likelihood, Mean Power, Multi-Band Covariance Analysis, Multi-Channel Covariance Analysis, and Multivariate Linear Regression.

In an example, this system can further comprise one or more components selected from the group consisting of: pollution sensor, thermal energy sensor, blood pressure sensor, conductive fabric, energy transducer to generate energy from body motion or kinetic energy, keypad, power source, thermistor, blood reservoir, conductivity sensor, energy transducer to generate energy from body thermal energy, magnetic field sensor, pressure sensor, thermocouple, buzzer, display screen, global positioning system (GPS), mobile phone, and speaker.

In an example data from wearable sensors can be analyzed using an analytical method selected from the group consisting of: Multivariate Logit, Multivariate Regression, Naive Bayes Classifier, Neural Network, Non-Linear Programming, Non-negative Matrix Factorization (NMF), Power Spectral Density, Power Spectrum Analysis, Principal Components Analysis (PCA), Probit Model, and Quadratic Minimum Distance Classifier.

In an example, this system can further comprise one or more components selected from the group consisting of: wireless data receiver, buttons, digital memory, gesture recognition interface, microprocessor, sound-emitting member, wireless communication module, amino acid sensor, cell phone, electromagnetic conductivity sensor, home electronics portal, oximetry sensor, AR eyewear, chemical sensor, electromagnetic energy emitter, home thermostat, pH level sensor, action potential sensor, caloric intake monitor, glucose sensor, motion sensor, spectrophotometer, altitude sensor, capnography sensor, electrocardiographic (ECG) sensor, and Hall-effect sensor.

In an example data from wearable sensors can be analyzed using an analytical method selected from the group consisting of: Random Forest (RF), Random Forest Analysis (RFA), Regression Model, Signal Amplitude (SA), Signal Averaging, Signal Decomposition, Sine Wave Compositing, Singular Value Decomposition (SVD), Spine Function, Support Vector and/or Machine (SVM), Time Domain Analysis, Time Frequency Analysis, Time Series Model, Trained Bayes Classifier, Variance, Waveform Identification, Wavelet Analysis, and Wavelet Transformation.

In an example, this system can further comprise one or more components selected from the group consisting of: neural impulse sensor, ballistocardiographic sensor, chemoreceptor, electromagnetic impedance sensor, humidity sensor, photodiode, strain gauge, ambient humidity sensor, carbon dioxide level, electrochemical sensor, heart rate monitor, olfactory sensor, ambient noise sensor, cardiopulmonary function sensor, electrogoniometer, hemoencephalography (HEG) sensor, optoelectronic sensor, brain-to-computer interface (BCI), and dial.

In an example, this system can further comprise one or more components selected from the group consisting of: food consumption sensor, microfluidic sensor, smart phone, voice recognition interface, air quality sensor, capacitance hygrometry sensor, electric motor, goniometer, motor, speech recognition interface, wireless data transmitter, brain activity sensor, data processor, environmental oxygen level sensor, Micro Electro Mechanical System (MEMS), pulse rate sensor, touch screen, ambient light sensor, and carbon monoxide sensor. In an example, this system can further comprise one or more components selected from the group consisting of: electroencephalographic (EEG) sensor, heart rate variability sensor, one-way valve, blood volume sensor, control unit, energy transmitted through inductively-coupled coils, magnetometer, pulse oximetry sensor, and thrombus-catching net.

FIGS. 1 through 3 are now discussed in detail. Relevant example and component variations discussed thus far can be applied to them, but are not repeated in the narratives accompanying each figure in order avoid narrative redundancy. FIG. 1 shows a semi-transparent view of an example of a closed loop system for human circulatory assistance comprising: a wearable device which is worn by a person, wherein the wearable device collects data on a biometric parameter; and an implanted cardiac pacemaker, wherein operation of the implanted cardiac pacemaker is controlled and/or adjusted based on analysis of the data on the biometric parameter. Specifically, FIG. 1 shows a semi-transparent view of an example of closed loop system for human circulatory assistance comprising: wrist-worn band 109, light-energy emitter 110, light-energy receiver 111, data processor 107, power source 108, first data transmitter and/or receiver 106, wireless electromagnetic transmission 105, second data transmitter and/or receiver 104, implanted cardiac pacemaker 103, and implanted cardiac pacemaker lead 102. FIG. 1 also shows heart 101 of a person in Vitruvian Man body position (ala Da Vinci) in order to show anatomical context. Relevant example and component variations discussed elsewhere in this disclosure and in priority-linked disclosures can also be applied to this example, but are not repeated here to avoid narrative redundancy.

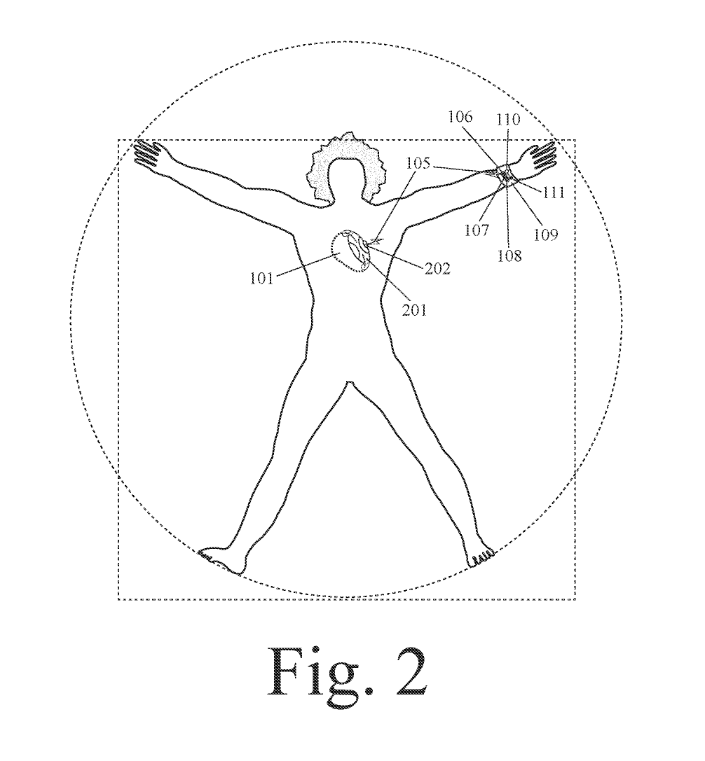

FIG. 2 shows a semi-transparent view of an example of a closed loop system for human circulatory assistance comprising: a wearable device which is worn by a person, wherein the wearable device collects data on a biometric parameter; and an implanted central blood pump, wherein operation of the implanted central blood pump is adjusted based on analysis of the data on the biometric parameter which is collected by the wearable device. Specifically, FIG. 2 shows a semi-transparent view of an example of closed loop system for human circulatory assistance comprising: wrist-worn band 109, light-energy emitter 110, light-energy receiver 111, data processor 107, power source 108, first data transmitter and/or receiver 106, wireless electromagnetic transmission 105, second data transmitter and/or receiver 202, and implanted central blood pump 201. FIG. 2 also shows heart 101 of a person in Vitruvian Man body position (ala Da Vinci) in order to show anatomical context. Relevant example and component variations discussed elsewhere in this disclosure and in priority-linked disclosures can also be applied to this example, but are not repeated here to avoid narrative redundancy.

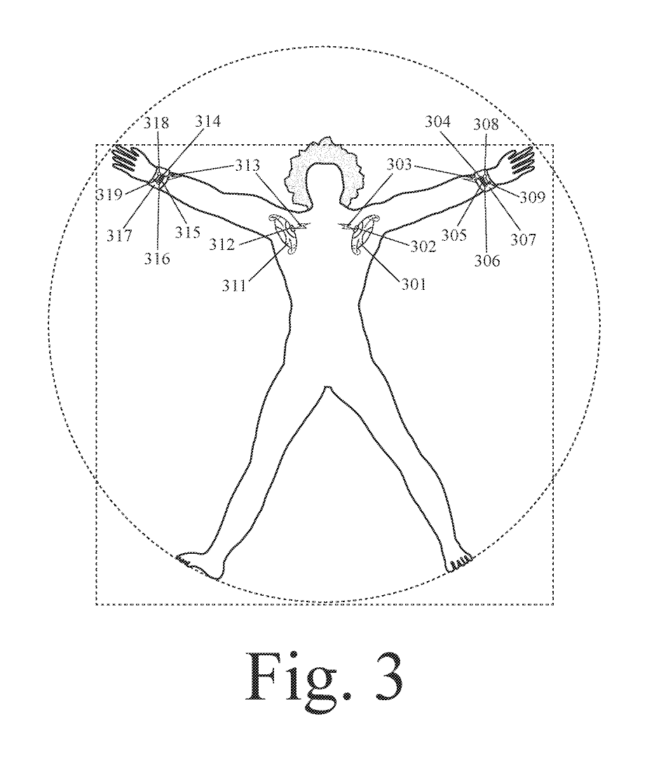

FIG. 3 shows a semi-transparent view of an example of a closed loop system for human circulatory assistance comprising: a first wearable device which is worn by a person on a first external location of the person's body, wherein the first wearable device collects data concerning a biometric parameter from the first external location; a second wearable device which is worn by the person on a second external location of the person's body, wherein the second wearable device collects data on the biometric parameter from the second external location; a first implanted non-central blood pump, wherein the first implanted non-central blood pump selectively increases blood flow to the first external location of the person's body based on the value of the biometric parameter at the first external location; and a second implanted non-central blood pump, wherein the second implanted non-central blood pump selectively increases blood flow to the second external location of the person's body based on the value of the biometric parameter at the second external location.

Specifically, FIG. 3 shows a semi-transparent view of an example of a closed loop system for human circulatory assistance comprising: first wrist-worn band 307, first light-energy emitter 308, first light-energy receiver 309, first data processor 305, first power source 304, first data transmitter and/or receiver 306, first wireless electromagnetic transmission 303, second data transmitter and/or receiver 302, first implanted central blood pump 301, second wrist-worn band 317, second light-energy emitter 318, second light-energy receiver 319, second data processor 315, second power source 314, third data transmitter and/or receiver 316, second wireless electromagnetic transmission 313, fourth data transmitter and/or receiver 312, and second implanted central blood pump 311. FIG. 3 also shows heart 101 of a person in Vitruvian Man body position (ala Da Vinci) in order to show anatomical context. Relevant example and component variations discussed elsewhere in this disclosure and in priority-linked disclosures can also be applied to this example, but are not repeated here to avoid narrative redundancy.

In an example, a closed loop system for human circulatory assistance can comprise: a wearable device, wherein the wearable device further comprises a light-energy emitter, a light-energy receiver, a data processor, and a power source; wherein the wearable device collects data on a biometric parameter; and an implanted cardiac pacemaker; wherein operation of the implanted cardiac pacemaker is controlled and/or adjusted based on analysis of the data on the biometric parameter.

In an example, a closed loop system for human circulatory assistance can comprise: a wearable device which is worn by a person; wherein the wearable device further comprises a light-energy emitter, a light-energy receiver, a data processor, a power source, and a first data transmitter and/or receiver; wherein light energy from the light-energy emitter is transmitted through or reflected from the person's body tissue and/or fluid before it reaches the light-energy receiver; and wherein a change in the spectrum of light energy received by the light-energy receiver due to transmission of the light energy through body tissue and/or fluid or reflection of the light energy from body tissue and/or fluid is analyzed to estimate a value of a biometric parameter concerning the person's body; and an implanted cardiac pacemaker and a second data transmitter and/or receiver which are implanted in the person's body, wherein operation of the implanted cardiac pacemaker is controlled and/or adjusted based on the estimated value of the biometric parameter.

In an example, a closed loop system for human circulatory assistance can comprise: a finger ring, smart watch, wrist band, ankle band, ear ring, or smart sock which is worn by a person; wherein the finger ring, smart watch, wrist band, ankle band, ear ring, or smart sock further comprises a light-energy emitter, a light-energy receiver, a data processor, a power source, and a first data transmitter and/or receiver; wherein light energy from the light-energy emitter is transmitted through or reflected from the person's body tissue and/or fluid before it reaches the light-energy receiver; and wherein a change in the spectrum of light energy received by the light-energy receiver due to transmission of the light energy through body tissue and/or fluid or reflection of the light energy from body tissue and/or fluid is analyzed to estimate the person's body oxygenation level; and an implanted cardiac pacemaker and a second data transmitter and/or receiver which are implanted in the person's body, wherein operation of the implanted cardiac pacemaker is controlled and/or adjusted based on the person's body oxygenation level.

In an example, a closed loop system for human circulatory assistance can comprise: a wearable device, wherein the wearable device further comprises a light-energy emitter, a light-energy receiver, a data processor, and a power source; wherein the wearable device collects data on a biometric parameter; and an implanted central blood pump; wherein operation of the implanted central blood pump is controlled and/or adjusted based on analysis of the data on the biometric parameter.

In an example, a closed loop system for human circulatory assistance can comprise: a wearable device which is worn by a person; wherein the wearable device further comprises a light-energy emitter, a light-energy receiver, a data processor, a power source, and a first data transmitter and/or receiver; wherein light energy from the light-energy emitter is transmitted through or reflected from the person's body tissue and/or fluid before it reaches the light-energy receiver; and wherein a change in the spectrum of light energy received by the light-energy receiver due to transmission of the light energy through body tissue and/or fluid or reflection of the light energy from body tissue and/or fluid is analyzed to estimate a value of a biometric parameter concerning the person's body; and an implanted central blood pump and a second data transmitter and/or receiver which are implanted in the person's body, wherein operation of the implanted central blood pump is controlled and/or adjusted based on the estimated value of the biometric parameter.

In an example, a closed loop system for human circulatory assistance can comprise: a finger ring, smart watch, wrist band, ankle band, ear ring, or smart sock which is worn by a person; wherein the finger ring, smart watch, wrist band, ankle band, ear ring, or smart sock further comprises a light-energy emitter, a light-energy receiver, a data processor, a power source, and a first data transmitter and/or receiver; wherein light energy from the light-energy emitter is transmitted through or reflected from the person's body tissue and/or fluid before it reaches the light-energy receiver; and wherein a change in the spectrum of light energy received by the light-energy receiver due to transmission of the light energy through body tissue and/or fluid or reflection of the light energy from body tissue and/or fluid is analyzed to estimate the person's body oxygenation level; and an implanted central blood pump and a second data transmitter and/or receiver which are implanted in the person's body, wherein operation of the implanted central blood pump is controlled and/or adjusted based on the person's body oxygenation level.

In an example, a closed loop system for human circulatory assistance comprising: a first wearable device which is worn by a person on a first external location of the person's body; wherein the first wearable device further comprises a first light-energy emitter, a first light-energy receiver, a first data processor, and a first power source; and wherein the first wearable device collects data on a biometric parameter from the first location; a second wearable device which is worn by a person on a second external location of the person's body; wherein the second wearable device further comprises a second light-energy emitter, a second light-energy receiver, a second data processor, and a second power source; and wherein the second wearable device collects data on the biometric parameter from the second location; a first implanted non-central blood pump, wherein the first implanted non-central blood pump selectively increases blood flow to the first external location of the person's body based on the value of the biometric parameter at the first external location; and a second implanted non-central blood pump, wherein the second implanted non-central blood pump selectively increases blood flow to the second external location of the person's body based on the value of the biometric parameter at the second external location.

In an example, a closed loop system for human circulatory assistance can comprise: (1) a first wearable device which is worn by a person on a first external location of the person's body; wherein the wearable device further comprises a first light-energy emitter, a first light-energy receiver, a first data processor, a first power source, and a first data transmitter and/or receiver; wherein light energy from the first light-energy emitter is transmitted through or reflected from the person's body tissue and/or fluid before it reaches the first light-energy receiver; and wherein a change in the spectrum of light energy received by the first light-energy receiver due to transmission of the light energy through body tissue and/or fluid or reflection of the light energy from body tissue and/or fluid is analyzed to estimate a first value of a biometric parameter concerning the person's body; (2) a second wearable device which is worn by a person on a second external location of the person's body; wherein the wearable device further comprises a second light-energy emitter, a second light-energy receiver, a second data processor, a second power source, and a second data transmitter and/or receiver; wherein light energy from the second light-energy emitter is transmitted through or reflected from the person's body tissue and/or fluid before it reaches the second light-energy receiver; and wherein a change in the spectrum of light energy received by the second light-energy receiver due to transmission of the light energy through body tissue and/or fluid or reflection of the light energy from body tissue and/or fluid is analyzed to estimate a second value of a biometric parameter concerning the person's body; (3) a first implanted non-central blood pump, wherein the first implanted non-central blood pump selectively increases blood flow to the first external location of the person's body based on the first value of the biometric parameter; and (4) a second implanted non-central blood pump, wherein the second implanted non-central blood pump selectively increases blood flow to the second external location of the person's body based on the second value of the biometric parameter.

In an example, a closed loop system for human circulatory assistance can comprise: (1) a first finger ring, smart watch, wrist band, ankle band, ear ring, or smart sock which is worn by a person on a first external location of the person's body; wherein the finger ring, smart watch, wrist band, ankle band, ear ring, or smart sock further comprises a first light-energy emitter, a first light-energy receiver, a first data processor, a first power source, and a first data transmitter and/or receiver; wherein light energy from the first light-energy emitter is transmitted through or reflected from the person's body tissue and/or fluid before it reaches the first light-energy receiver; and wherein a change in the spectrum of light energy received by the first light-energy receiver due to transmission of the light energy through body tissue and/or fluid or reflection of the light energy from body tissue and/or fluid is analyzed to estimate a first value of the person's body oxygenation level; (2) a second finger ring, smart watch, wrist band, ankle band, ear ring, or smart sock which is worn by a person on a second external location of the person's body; wherein the finger ring, smart watch, wrist band, ankle band, ear ring, or smart sock further comprises a second light-energy emitter, a second light-energy receiver, a second data processor, a second power source, and a second data transmitter and/or receiver; wherein light energy from the second light-energy emitter is transmitted through or reflected from the person's body tissue and/or fluid before it reaches the second light-energy receiver; and wherein a change in the spectrum of light energy received by the second light-energy receiver due to transmission of the light energy through body tissue and/or fluid or reflection of the light energy from body tissue and/or fluid is analyzed to estimate a second value of the person's body oxygenation level; (3) a first implanted non-central blood pump, wherein the first implanted non-central blood pump selectively increases blood flow to the first external location of the person's body based on the first value of the person's body oxygenation level; and (4) a second implanted non-central blood pump, wherein the second implanted non-central blood pump selectively increases blood flow to the second external location of the person's body based on the second value of the person's body oxygenation level.

I claim:

1. A closed loop system for human circulatory assistance comprising:
a wearable device, wherein the wearable device further comprises a light-energy emitter, a light-energy receiver, a data processor, and a power source; wherein the wearable device collects data on a biometric parameter; and
an implanted cardiac pacemaker; wherein operation of the implanted cardiac pacemaker is controlled and/or adjusted based on analysis of the data on the biometric parameter.

2. A closed loop system for human circulatory assistance comprising:
a wearable device, wherein the wearable device further comprises a light-energy emitter, a light-energy receiver, a data processor, and a power source; wherein the wearable device collects data on a biometric parameter; and
an implanted central blood pump; wherein operation of the implanted central blood pump is controlled and/or adjusted based on analysis of the data on the biometric parameter.

3. A closed loop system for human circulatory assistance comprising:
a first wearable device which is worn by a person on a first external location of the person's body; wherein the first wearable device further comprises a first light-energy emitter, a first light-energy receiver, a first data processor, and a first power source; and wherein the first wearable device collects data on a biometric parameter from the first location;
a second wearable device which is worn by the person on a second external location of the person's body; wherein the second wearable device further comprises a second light-energy emitter, a second light-energy receiver, a second data processor, and a second power source; and wherein the second wearable device collects data on the biometric parameter from the second location;
a first implanted non-central blood pump, wherein the first implanted non-central blood pump selectively increases blood flow to the first external location of the person's body based on the value of the biometric parameter at the first external location; and
a second implanted non-central blood pump, wherein the second implanted non-central blood pump selectively increases blood flow to the second external location of the person's body based on the value of the biometric parameter at the second external location.
